(19) United States
(12) Patent Application Publication
(10) Pub. No.: US 2022/0259212 A1
(43) Pub. Date: Aug. 18, 2022

(54) INHIBITORS OF INDOLEAMINE 2,3-DIOXYGENASE AND/OR TRYPTOPHAN 2,3-DIOXYGENASE

(71) Applicant: Idorsia Pharmaceuticals Ltd, Allschwil (CH)

(72) Inventors: Christoph BOSS, Allschwil (CH); Sylvaine CREN, Allschwil (CH); Thierry KIMMERLIN, Allschwil (CH); Carina LOTZ-JENNE, Allschwil (CH); Julien POTHIER, Allschwil (CH); Naomi TIDTEN-LUKSCH, Allschwil (CH)

(21) Appl. No.: 17/626,074

(22) PCT Filed: Jul. 10, 2020
(86) PCT No.: PCT/EP2020/069609

§ 371 Date: Jan. 10, 2022

(30) Foreign Application Priority Data
Jul. 11, 2019 (EP) 19185840.6

(51) Int. Cl. C07D 487/04; C07D 471/04

(52) U.S. Cl. CPC C07D 487/04; C07D 471/04

(57) ABSTRACT

The present invention relates to compounds of Formula (I) inhibiting indoleamine 2,3-dioxygenase (IDO) and/or tryptophan 2,3-dioxygenase (TDO) enzymes. Further, their synthesis and their use as medicaments in the treatment of inter alia cancer is disclosed.

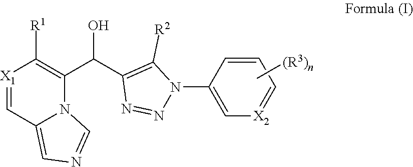

The present invention relates to compounds represented by Formula (I), or pharmaceutically acceptable salts thereof, and their use as active ingredients in medicine. The invention further concerns a process for the preparation of said compounds, pharmaceutical compositions containing one or more of said compounds, and their use, either alone or in combination with other active compounds or therapies as modulators of the activity of indoleamine 2,3-dioxygenase (IDO; also known as IDO1) and/or tryptophan 2,3-dioxygenase (TDO) enzymes.

The enzymes IDO and TDO catalyze the first and rate limiting step in the kynurenine pathway which is responsible for more than 95% of the degradation of the essential amino acid tryptophan (TRP). The catabolism of TRP is a central pathway maintaining the immunosuppressive microenvironment in many types of cancers. The kynurenine pathway is also involved in physiological functions such as behavior, sleep, thermo-regulation and pregnancy.

The classic concept proposes that tumor cells or myeloid cells in the tumor microenvironment or draining lymph nodes express high levels of IDO resulting in the depletion of TRP and accumulation of TRP metabolites in the local microenvironment and subsequent inhibition of T cell responses. This IDO-centered concept is supported by numerous preclinical studies in models of tumor immunity, autoimmunity, infection, and allergy. More recent preclinical studies propose an alternative route of TRP degradation in tumors via the enzyme TDO. It has been suggested that targeting TDO may complement IDO inhibition. Thus, inhibition of IDO and/or TDO enzymes may be utilized in preventing and/or treating cancers. Moreover, a wide spectrum of further diseases and/or disorders notably neurological conditions, infectious and other diseases may be prevented and/or treated by targeting IDO and/or TDO.

Several IDO and/or TDO inhibitors are described in WO2010005958, WO2011037780, WO2012142237, WO2015173764, WO2016073770 and some have been clinically tested as anticancer agents either alone or in combination with other compounds/therapies. WO2016161960, WO2017134555, WO2018036414, WO2017007700, WO2017189386, WO2017133258, CN107556244, WO2018057973, WO2018136887, WO2018171602, WO2018054365, WO2019034725, WO2019076358, WO2019040102, and WO2019138107 disclose certain heterocyclic derivatives which may be used for inhibiting IDO and/or TDO enzymes.

Studying human tumor samples for expression of TDO2 gene revealed significant expression in 41% of bladder carcinomas, 50% of melanomas and 100% of hepatocarcinomas (Pilotte et al.; Proc Natl Acad Sci. 2012, 109(7):2497-502). Moreover, TDO is expressed constitutively in human glioblastomas. Besides the suppression of anti-tumor immune responses, TDO-derived kynurenine (KYN) has been shown to have a tumor cell autonomous effect in glioblastoma, promoting tumor-cell survival and motility through the aryl hydrocarbon receptor (AHR) in an autocrine fashion. The TDO-AHR pathway in human brain tumors was found to be associated with malignant progression and poor survival. Elevated expression of TDO has also been observed in clinical specimens of Triple Negative Breast Cancer (TNBC) and was associated with increased disease grade, estrogen receptor negative status and shorter overall survival. KYN production mediated by TDO in TNBC cells was sufficiently to activate the AhR promoting anoikis resistance, migration, and invasion (D'Amato et al.; Cancer Res. 2015, 75(21):4651-64).

TDO expression has been detected in other cancer indications, such as for example renal cell carcinoma, mesothelioma, neuroblastoma, leukemia, lung carcinoma (NSCLC), head&neck carcinoma, colorectal carcinoma, sarcoma, astrocytoma, myeloma, and pancreatic carcinoma (Pilotte et al.; Proc Natl Acad Sci. 2012, 109(7):2497-502).

IDO expression levels in patient tumor samples varied slightly with the use of different antibodies reflecting the potential for alternative splice variants and/or post-translational modifications. Overall, IDO expression was found in a large fraction (>50%) of human tumors comprising tumor cells, endothelial cells, and stromal cells in proportions that varied depending on the tumor type (Uyttenhove et al.; Nat Med. 2003, 9(10):1269-74). Tumors showing the highest proportions of IDO-immunolabeled samples were carcinomas of the endometrium and cervix, followed by kidney, lung, and colon. This hierarchy of IDO expression was confirmed by gene expression data mined from The Cancer Genome Atlas database (Theate et al.; Cancer Immunol Res. 2015, 3(2):161-72). In most studies, high expression of IDO in the tumor or draining lymph nodes has been an adverse prognostic factor. Tumor in this category include melanoma, colon cancer, brain tumors, ovarian cancer, acute myelogenous leukemia, endometrial cancer, high-grade osteosarcoma and a number of others (Munn and Mellor; Trends in Immunol. 2016, 37(3): 193-207). In a smaller number of tumor types, IDO expression appears to be induced or ‘reactive’—that is associated with increased T cell infiltration and inflammation. In this situation, upregulation of IDO may be a proxy for a stronger spontaneous anti-tumor immune response, and thus associated with more favorable prognosis. However, even in these immune-responsive patients, the IDO itself is not beneficial, and the patient might do even better if IDO were blocked.

Because of the differences observed for IDO expression levels in patient samples using different antibodies, measuring IDO activity by determining concentrations of KYN and TRP in the serum might be more meaningful. Indeed, increased KYN/TRP ratios have been detected in sera from cancer patients compared to normal volunteers (Liu et al.; Blood. 2010, 115(17):3520-30). The KYN/TRP ratio was recently validated as a prognostic tool in cervical cancer patients whereby low TRP levels indicated a tumor size greater than 4 cm and metastatic spread to the lymph node (Ferns et al.; Oncoimmunology. 2015, 4(2):e981457). Accordingly, high KYN/TRP ratios in patient sera were associated with lymph node metastasis, FIGO stage, tumor size, parametrial invasion and poor disease-specific survival, further suggesting the relevance of IDO targeting based on a TRP catabolic signature. Moreover, serum KYN/TRP ratio was a significantly independent detrimental prognostic factor in patients with adult T-cell leukemia/lymphoma (Zhai et al.; Clin Cancer Res. 2015, 21(24):5427-33).

In preclinical models transfection of immunogenic tumor cells with recombinant IDO prevented their rejection in mice (Uyttenhove et al.; Nat Med. 2003, 9(10):1269-74). While ablation of IDO expression led to a decrease in the incidence and growth of 7,12-dimethylbenz[a]anthracene-induced premalignant skin papillomas (Muller et al.; Proc Natl Acad Sci USA. 2008, 105(44):17073-8).

In preclinical models of B16 melanoma overexpressing IDO and 4T1 breast cancer, IDO expression by tumor cells promoted tumor growth through the recruitment and activation of myeloid-derived suppressor cells (MDSC) and resistance to checkpoint blockade using anti-CTLA-4 and anti-PD-1. In the same study, it was also noted that IDO expression in human melanoma tumors is strongly associated with MDSC infiltration (Holmgaard et al.; Cell Rep. 2015, 13(2):412-24).

Imatinib, a small-molecule receptor tyrosine kinase inhibitor targeting KIT (CD117), used for treatment of gastrointestinal stromal tumor (GIST), has been shown to modulate the KYN pathway. In a mouse model of GIST, imatinib therapy produced a number of immunological responses by reducing tumor cell expression of IDO. To test the hypothesis that the immune effects of imatinib are partially mediated by its reduction of IDO expression, GIST mice were treated with a cocktail of KYN pathway metabolites-KYN, 3-hydroxyanthranilic acid (3-HAA), and 3-hydroxykynurenine (3-HK), designed to simulate a system with competent IDO activity. The antitumor effects of imatinib were diminished by coadministration of the TRP metabolite cocktail. However, the antitumor effects of imatinib were not increased by co-administration of the IDO inhibitor 1-methyl-tryptophan (1-MT), consistent with the hypothesis that both agents are impacting the same pathway (Balachandran et al.; Nat Med. 2011, 17(9): 1094-100).

It has been shown that TDO expression by tumors prevented their rejection by immunized mice and systemic treatment with a TDO inhibitor restored the ability of mice to reject the TDO-expressing tumors (Pilotte et al.; Proc Natl Acad Sci. 2012, 109(7):2497-502). In a transplantable model of glioma, TDO expression in tumor cells promoted tumor growth while TDO knockdown decreased tumor incidence (Opitz et al.; Nature 2011, 478(7368):197-203).

IDO inhibitors have been found to suppress TRP metabolism in vivo in tumors and blood which was accompanied by a slowdown of tumor outgrowth in experimental models of colorectal cancer (Lin et al.; J Med Chem. 2016, 59(1):419-30; Koblish et al.; Mol Cancer Ther. 2010, 9(2):489-98; Kraus et al.; AACR Annual Meeting (Apr. 16-20, New Orleans, La.) 2016: abstract 4863; Wise et al.; AACR Annual Meeting (Apr. 16-20, New Orleans, La.) 2016: abstract 5115; Liu et al.; AACR Annual Meeting (Apr. 16-20, New Orleans, La.) 2016: abstract 4877), pancreatic cancer (Koblish et al.; Mol Cancer Ther. 2010, 9(2):489-98), melanoma (Yue et al.; J Med Chem. 2009, 52(23):7364-7), lung (Yang et al.; J Med Chem. 2013, 56(21):8321-31), breast cancer (Holmgaard et al.; Cell Rep. 2015, 13(2):412-24), glioma (Hanihara et al.; J Neurosurg. 2016, 124(6):1594-601).

1-Methyl-Tryptophan (1-MT) augmented the effect of chemotherapy in mouse models of transplantable melanoma (B16) and transplantable and autochthonous breast cancer (4T1) (Hou et al.; Cancer Res. 2007, 67(2):792-801). Furthermore, 1-MT enhanced chemo-radiation therapy to prolong survival in mice bearing intracranial glioblastoma tumors (GL-261). In this context inhibition of IDO allowed chemo-radiation to trigger widespread complement deposition at sites of tumor growth. IDO-blockade led to upregulation of VCAM-1 on vascular endothelium within the tumor microenvironment. Mice genetically deficient in complement component C3 lost all of the synergistic effects of IDO-blockade on chemo-radiation-induced survival (Li et al.; Journal Immunother Cancer. 2014, 2:21). IDO expression is induced in the tumor epithelium of a significant number of patients with pancreatic cancer after GVAX (irradiated, GM-CSF-secreting, allogeneic PDAC) vaccination. GVAX vaccination combined with IDO inhibition increases survival in a preclinical model of pancreatic cancer and with the combination of cyclophosphamide, GVAX vaccine, IDO inhibition and PD-L1 blockade all mice survived (Zheng, John Hopkins School of Medicine; ITOC3 conference (Mar. 21-23, Munich, Germany) 2016). In this context, vaccination combined with increasing doses of anti-OX40 has also been shown to induce IDO in the TC1 tumor model and inhibition of IDO by 1-MT showed synergistic effects with anti-OX40 and vaccination in the same model (Khleif, Georgia Cancer Center; ITOC3 conference (Mar. 21-23, Munich, Germany) 2016). Moreover, IDO inhibitor epacadostat has been shown to enhance the effect of anti-OX40 and anti-GITR in preclinical models (Koblish et al.; AACR Annual Meeting (Apr. 1-5, Washington D.C.) 2017: abstract #2618).

The IDO/TDO dual inhibitor NLG919 enhanced the antitumor responses of naïve, resting adoptively transferred pmel-1 cells to vaccination with cognate human gp100 peptide in the B16F10 tumor model. The effect was additive with chemotherapy and even more pronounced once chemotherapy was combined with indoximod/anti-PD-1 (Mautino et al.; AACR Annual Meeting (Apr. 5-9, San Diego, Calif.) 2014: abstract 5023). Along these lines, improved depth and duration of tumor growth inhibition was detected when NLG-919 was combined with anti-PD-L1 in the EMT-6 mouse model (Spahn et al.; Journal for ImmunoTherapy of Cancer 2015, 3 (Suppl 2): P303).

IDO-selective inhibitors have been shown to enhance chemotherapy in the tumor mouse models: An IDO-selective inhibitor from 10Met Pharma enhances chemotherapy (gemcitabine and abraxane) in the PAN02 model (Wise et al.; AACR Annual Meeting (Apr. 16-20, New Orleans, La.) 2016: abstract 5115).

In plasma and tumor tissue, anti-PD-L1 and anti-CTLA4 checkpoint blockade induce IDO activity, while the combination of an IDO-selective inhibitor (PF-06840003) and anti-PD-L1 treatment resulted in significant tumor growth inhibition in the CT-26 syngeneic mouse colon tumor model (Kraus et al.; AACR Annual Meeting (Apr. 16-20, New Orleans, La.) 2016: abstract 4863). In another study, doublet therapies using either anti-CTLA-4, anti-PD-L1 and/or an IDO inhibitor showed synergistic retardation of tumor outgrowth in the B16(SIY) melanoma mouse model (Sprenger et al.; J Immunother Cancer. 2014, 2:3). The major biologic correlate to this improved efficacy was restored IL-2 production and proliferation of tumor-infiltrating CD8 T cells. Functional restoration did not require new T cell migration to the tumor. In yet another study, inhibition of IDO by 1-MT in combination with therapies targeting immune checkpoints such as CTL-4, PD-1/PD-L1, and GITR synergize to control tumor outgrowth and enhance overall survival in the B16-F10 and 4T1 tumor mouse models (Holmgaard et al.; J Exp Med. 2013, 210(7):1389-402). In an orthotopic glioma model triple treatment with anti-CTLA-4, anti-PD-L1 and 1-MT as well as the combination of Epacadostat and anti-PD-1 resulted in a highly effective durable survival advantage (Wainwright et al.; Clin Cancer Res. 2014, 20(20):5290-301; Reardon et al.; AACR Annual Meeting (Apr. 1-5, Washington D.C.) 2017: abstract 572). The concept of targeting IDO in combination with checkpoint blockade has been investigated in several clinical trials (NCT02752074, NCT02658890, NCT02327078, NCT02318277, NCT02178722, NCT02471846, NCT02298153).

Intra-tumoral treatment with a TLR9 agonist was shown to induce IDO expression in treated and distant tumors and the combination of an IDO inhibitor with the same TLR9 agonist showed additive anti tumor effects in the CT-26 syngeneic mouse colon tumor model (Wang et al.; AACR Annual Meeting (Apr. 16-20, New Orleans, La.) 2016: abstract 3847).

High IDO expression induces recruitment of immunosuppressive MDSC to tumors in several mouse models. CSF-1R was found to be expressed on MDSCs and CSF-1R blockade to inhibit intratumoral MDSCs. Accordingly, inhibiting IDO with D-1-MT was shown to synergize with CSF-1R blockade in the B16 model overexpressing IDO (Holmgaard et al.; EBioMedicine 2016, 6:50-8).

There is experimental evidence that IDO inhibition also improves the therapeutic response to chimeric antigen receptor (CAR) T cell therapy in B cell lymphoma. In a mouse model of B cell lymphoma IDO expression in tumor cells suppress CD19 CAR T cell therapy through the action of TRP metabolites. The treatment with the IDO inhibitor 1-MT restored tumor control by CAR T cells in this model (Ninomiya et al.; Blood, 2015, 125(25):3905-16).

DNA nanoparticles can induce IDO via a pathway dependent on the stimulator of interferon genes (STING) sensor of cytosolic DNA. Accordingly, STING agonists can induce IDO and promote tolerogenic responses. This scenario has been studied in preclinical models using tumors with low and high antigenicity. In tumors exhibiting low antigenicity IDO activation by STING is predominant and overcomes STING/IFN immunogenic responses while in tumors with high antigenicity the STING/IFN signaling rather potentiates immunogenic responses and fails to induce IDO. Overall these data suggest that IDO inhibition can enhance the anti-tumor response to STING agonists particularly in tumors with low antigenicity (Lemos et al.; Cancer Res. 2016, 76(8):2076-81).

Given the role of the JAK-STAT (signal transducer and activator of transcription) signalling system in mediating interferon-γ-induced IDO expression, it is obvious to combine IDO inhibitors with JAK/STAT inhibitors. A clinical trial on this treatment concept has been reported (NCT02559492).

In the central nervous system both fates of TRP which act as a precursor to KYN and serotonin are pathways of interest and importance. Metabolites produced by the KYN pathway have been implicated to play a role in the pathomechanism of neuroinflammatory and neurodegenerative disorder such as Huntington's disease. The first stable intermediate from the KYN pathway is KYN. Subsequently, several neuroactive intermediates are generated. They include Kynurenic acid (KYNA), 3-Hydroxykynurenine (3-HK), and Quinolinic acid (QUIN). 3-HK and QUIN are neurotoxic by distinct mechanisms; 3-HK is a potent free-radical generator (Thevandavakkam et al.; CNS Neurol Disord. Drug Targets. 2010, 9(6):791-800; Ishii et al.; Arch Biochem Biophys. 1992, 294(2):616-622; Hiraku et al.; Carcinogenesis. 1995, 16(2):349-56), whereas QUIN is an excitotoxic N-methyl-D-aspartate (NMDA) receptor agonist (Stone and Perkins; Eur J Pharmacol. 1981, 72(4):411-2; Schwarcz et al; Science. 1983, 219(4582):316-8). KYNA, on the other hand, is neuroprotective through its antioxidant properties and antagonism of both the a7 nicotinic acetylcholine receptor and the glycine coagonist site of the NMDA receptor (Vecsei and Beal; Brain Res Bull. 1990, 25(4):623-7; Foster et al.; Neurosci Lett. 1984, 48(3):273-8; Carpenedo et al.; Eur J Neurosci. 2001, 13(11):2141-7; Goda et al.; Adv. Exp. Med. Biol. 1999, 467:397-402). Changes in the concentration levels of TRP catabolites can shift the balance to pathological conditions. The ability to influence the metabolism towards the neuroprotective branch of the KYN pathway, i.e. towards KYNA synthesis, may be used in preventing neurodegenerative diseases.

In the CNS, the KYN pathway is present to varying extents in most cell types, infiltrating macrophages, activated microglia and neurons have the complete repertoire of KYN pathway enzymes. On the other hand, neuroprotective astrocytes and oligodendrocytes lack the enzyme, KYN 3-monooxygenase (KMO) and IDO-1 respectively, and are incapable of synthesizing the excitotoxin QUIN (Guillemin et al.; Redox Rep 2000, 5(2-3): 108-11; Lim et al.; International Congress Series. 2007, 1304: 213-7). TDO is expressed in low quantities in the brain, and is induced by TRP or corticosteroids (Salter and Pogson; Biochem J. 1985, 229(2): 499-504; Miller et al.; Neurobiol Dis. 2004, 15(3): 618-29). Given the role of TDO and IDO in the pathogenesis of several CNS disorders such as schizophrenia as well as the role of TDO in controlling systemic TRP levels, IDO and/or TDO inhibitors could be used to improve the outcomes of patients with a wide variety of CNS diseases and neurodegeneration.

IDO and/or TDO inhibitors may in addition be useful for the treatment of Amyotrophic lateral sclerosis (ALS) (or Lou Gehrig's disease). ALS results in the selective attacking and destruction of motor neurons in the motor cortex, brainstem and spinal cord. Although multiple mechanisms are likely to contribute to ALS, the KYN pathway activated during neuroinflammation is emerging as a contributing factor. Initial inflammation may inflict a nonlethal injury to motor neurons of individuals with a susceptible genetic constitution, in turn triggering a progressive inflammatory process which activates microglia to produce neurotoxic KYN metabolites that further destroy motor neurons. In the brain and spinal cord of ALS patients large numbers of activated microglia, reactive astrocytes, T cells and infiltrating macrophages have been observed (Graves et al.; Amyotroph Lateral Scler Other Motor Neuron Disord. 2004, 5(4):213-9; Henkel et al.; Ann Neurol. 2004, 55(2):221-35). These cells release inflammatory and neurotoxic mediators, among others IFN-γ, the most potent inducer of IDO (McGeer and McGeer; Muscle Nerve. 2002; 26(4):459-70). The neuronal and microglial expression of IDO is increased in ALS motor cortex and spinal cord (Chen et al.; Neurotox Res. 2010, 18(2):132-42). It has been proposed that the release of immune activating agents activates the rate-limiting enzyme of the KYN pathway, IDO, which generates metabolites such as the neurotoxin QUIN. Therefore, inhibition of IDO may reduce the synthesis of neurotoxic QUIN, which has been clearly implicated in the pathogenesis of ALS.

IDO and/or TDO inhibitors may in addition be useful for the treatment of Huntington's disease (HD). HD is a genetic autosomal dominant neurodegenerative disorder caused by expansion of the CAG repeats in the huntingtin (htt) gene. Patients affected by HD display progressive motor dysfunctions characterized by abnormality of voluntary and involuntary movements (choreoathetosis) and psychiatric and cognitive disturbances. In-life monitoring of metabolites within the KYN pathway provide one of the few biomarkers that correlates with the number of CAG repeats and hence the severity of the disorder (Forrest et al.; J Neurochem 2010, 112(1):112-22). Indeed, in patients with HD and HD model mice, 3-HK and QUIN levels are increased in the neostriatum and cortex. Moreover, KYNA levels are reduced in the striatum of patients with HD. Intrastriatal injection of QUIN in rodents reproduces behavioural and pathological features of HD (Sapko et al.; Exp Neurol. 2006 197(1):31-40). Importantly, TDO ablation in a Drosophila model of HD ameliorated neurodegeneration (Campesan et al.; Curr Biol. 2011; 21(11):961-6).

IDO and/or TDO inhibitors may in addition be useful for the treatment of Alzheimer's disease (AD). AD is an age-related neurodegenerative disorder characterised by neuronal loss and dementia. The histopathology of the disease is manifested by the accumulation of intracellular 3-amyloid (AR) and subsequent formation of neuritic plaques as well as the presence of neurofibrillary tangles in specific brain regions associated with learning and memory. The pathological mechanisms underlying this disease are still controversial, however, there is growing evidence implicating KYN pathway metabolites in the development and progression of AD. It has been shown that Aβ (1-42) can activate primary cultured microglia and induce IDO expression (Guillemin et al.; Redox Rep. 2002, 7(4):199-206; Walker et al.; J Leukoc Biol. 2006, 79:596-610). Furthermore, IDO over-expression and increased production of QUIN have been observed in microglia associated with the amyloid plaques in the brain of AD patients (Guillemin et al.; Neuropathol Appl Neurobiol. 2005, 31(4):395-404). QUIN has been shown to lead to tau hyperphosphorylation in human cortical neurons (Rahman et al.; PLOS One. 2009, 4(7):e6344). Thus, overexpression of IDO and over-activation of the KYN pathway in microglia are implicated in the pathogenesis of AD. There is also evidence for TDO involvement in Alzheimer's disease. TDO is upregulated in the brain of patients and AD mice models. Furthermore, TDO co-localizes with quinolinic acid, neurofibrillary tangles-tau and amyloid deposits in the hippocampus of AD patients (Wu et al.; PLOS One. 2013, 8(4):e59749). Preclinical evidence supports the use of KMO, TDO, IDO, and 3HAO inhibitors to offset the effects of neuroinflammation in AD. Moreover, other observations have demonstrated that the ratio of KYN/TRP is increased in the serum of AD patients (Widner et al.; J Neural Transm (Vienna). 2000, 107(3):343-53). In fly models of AD both genetic and pharmacological inhibition of TDO provides robust neuroprotection (Breda et al.; Proc Natl Acad Sci. 2016, 113(19):5435-40). Therefore, the KYN pathway is over-activated in AD by both TDO and IDO and may be involved in neurofibrillary tangle formation and associated with senile plaque formation.

IDO and/or TDO inhibitors may in addition be useful for the treatment of Parkinson's disease (PD). PD is a common neurodegenerative disorder characterised by loss of dopaminergic neurons and localized neuroinflammation. Parkinson's disease is associated with chronic activation of microglia (Gao and Hong; Trends Immunol. 2008, 29(8):357-65). Microglia activation release neurotoxic substances including reactive oxygen species (ROS) and proinflammatory cytokines such as INF-γ (Block et al.; Nat Rev Neurosci. 2007; 8(1):57-69), a potent activator of KYN pathway via induction of IDO expression. KYN pathway in activated microglia leads to upregulation of 3HK and QUIN. 3HK is toxic primarily as a result of conversion to ROS (Okuda et al.; J Neurochem. 1998; 70(1):299-307). The combined effects of ROS and NMDA receptor-mediated excitotoxicity by QUIN contribute to the dysfunction of neurons and their death (Stone and Perkins; Eur J Pharmacol. 1981, 72(4): 411-2; Braidy et al.; Neurotox Res. 2009, 16(1):77-86). However, picolinic acid (PIC) produced through KYN pathway activation in neurons, has the ability to protect neurons against QUIN-induced neurotoxicity, being a NMDA agonist (Jhamandas et al.; Brain Res. 1990, 529(1-2):185-91). Microglia can become overactivated, by proinflammatory mediators and stimuli from dying neurons and cause perpetuating cycle of further microglia activation microgliosis. Excessive microgliosis will cause neurotoxicity to neighbouring neurons and resulting in neuronal death, contributing to progression of Parkinson's disease. Therefore, PD is associated with an imbalance between the two main branches of the KYN pathway within the brain. KYNA synthesis by astrocytes is decreased and concomitantly, QUIN production by microglia is increased. Importantly, both genetic and pharmacological inhibition of TDO provided robust neuroprotection in a fly model of PD (Breda et al.; Proc Natl Acad Sci. 2016, 113(19):5435-40).

IDO and/or TDO inhibitors may in addition be useful for the treatment of Multiple sclerosis (MS). MS is an autoimmune disease characterized by inflammatory lesions in the white matter of the nervous system, consisting of a specific immune response to the myelin sheet resulting in inflammation and axonal loss (Trapp et al.; Curr Opin Neurol. 1999, 12: 295-302; Owens; Curr Opin Neurol. 2003, 16:259-265). Accumulation of neurotoxic KYN metabolites caused by the activation of the immune system is implicated in the pathogenesis of MS. QUIN was found to be selectively elevated in the spinal cords of rats with EAE, an autoimmune animal model of MS (Flanagan et al.; J Neurochem. 1995, 64: 1192-6). The origin of the increased QUIN in EAE was suggested to be the macrophages. QUIN is an initiator of lipid peroxidation and high local levels of QUIN near myelin may contribute to the demyelination in EAE and possibly MS. Interferon-β Ib (IFN-pib) induces KYN pathway metabolism in macrophages at concentrations comparable to those found in the sera of IFN-β treated patients, which may be a limiting factor in its efficacy in the treatment of MS (Guillemin et al.; J Interferon Cytokine Res. 2001, 21:1097-1101). After IFN-β administration, increased KYN levels and KYN/TRP ratio were found in the plasma of MS patients receiving IFN-β injection compared to healthy subjects indicating an induction of IDO by IFN-β (Amirkhani et al.; Eur. J. Neurol. 2005, 12, 625-31). IFN-pib, leads to production of QUIN at concentrations sufficient to disturb the ability of neuronal dendrites to integrate incoming signals and kill oligodendrocytes (Cammer et al.; Brain Res. 2001, 896: 157-160). In IFN-pib-treated patients concomitant blockade of the KYN pathway with an IDO/TDO inhibitor may improve its efficacy of IFN-pib.

A homolog of IDO (IDO2) has been identified that shares 44% amino acid homology with IDO, but its function is largely distinct from that of IDO (Ball et al., Gene 2007, 396(1):203-13; Yuasa et al., J Mol Evol 2007, 65(6):705-14. An IDO inhibitor may modulate IDO1 and/or IDO2. Current evidence reveals IDO2 to be an immunomodulatory enzyme that acts in B cells to modulate autoimmune disease. Although its enzymatic function is poorly characterized, the mechanism of immune modulation by IDO2 is distinct from its better-studied homolog, IDO1. IDO2 acts as a pro-inflammatory mediator in multiple models of autoimmune inflammatory disorders, including rheumatoid arthritis, Contact hypersensitivity, and Systemic lupus erythematosus (Merlo and Mandik-Nayak, Clinical Medicine Insights: Pathology 2016, 9(S1): 21-28). Because IDO2 is acting to promote inflammation, it may be a candidate for therapeutic targeting for treatment of these diseases, particularly in a co-therapeutic setting.

Most TRP is processed through the KYN pathway. A small proportion of TRP is processed to 5-HT and hence to melatonin, both of which are also substrates for IDO. It has long been known that amongst other effects acute TRP depletion can trigger a depressive episode and produces a profound change in mood even in healthy individuals. These observations link well with the clinical benefits of serotonergic drugs both to enhance mood and stimulate neurogenesis.

In recent years, the general view of the pathophysiology of schizophrenia (i.e., disturbances in dopamine [DA] transmission) has been expanded to also involve a glutamatergic dysfunction of the brain. Thus, clinical observations show that systemic administration of N-methyl-D-aspartate (NMDA) receptor antagonists (e.g., phencyclidine [PCP] and ketamine) evokes schizophrenia-like symptoms in healthy individuals and provokes symptoms in patients with schizophrenia (Holtze et al.; J Psychiatry Neurosci. 2012, 37(1):53-7). Furthermore, the glutamate deficiency theory has gained some support from genetic findings. A hypoglutamatergic state of the brain can also be achieved by elevation of the endogenous NMDA receptor antagonist KYNA. Indeed, altered brain level of KYNA and of KYNA-producing enzymes are found in the post-mortem brains of schizophrenic patients (Barry et al.; J Psychopharmacol. 2009, 23(3):287-94). In particular, elevated KYN and KYNA levels are found in the frontal cortex and an upregulation of the first step of the KYN pathway is observed in the anterior cingulate cortex of individuals with schizophrenia (Miller et al.; Brain Res. 2006, 1073-1074:25-37). However, other researchers have found that KYNA is decreased and 3-HAA is increased in schizophrenia (Miller et al.; Neurochem Int. 2008, 52(6):1297-303). The mechanism of elevation of KYN metabolites in schizophrenia has not been fully elucidated. Mechanisms include KMO polymorphisms and TDO upregulation (Miller et al.; Neurobiol Dis. 2004, 15(3):618-29). Therefore, IDO and/or TDO inhibitors may be useful for the treatment of schizophrenia.

IDO and/or TDO inhibitors may in addition be useful for the treatment of pain and depression. Pain and depression are frequently comorbid disorders. It has been shown that IDO plays a key role in this comorbidity. Recent studies have shown that IDO activity is linked to (a) decreased serotonin content and depression (Dantzer et al.; Nat Rev Neurosci. 2008, 9(1):46-56; Sullivan et al; Pain. 1992, 50(1):5-13) and (b) increased KYN content and neuroplastic changes through the effect of its derivatives such as quinolinic acid on glutamate receptors (Heyes et al.; Brain. 1992, 115(Pt5):1249-73).

In rats chronic pain induced depressive behaviour and IDO upregulation in the bilateral hippocampus. Upregulation of IDO resulted in the increased KYN/TRP ratio and decreased serotonin/TRP ratio in the bilateral hippocampus. Furthermore, IDO gene knockout or pharmacological inhibition of hippocampal IDO activity attenuated both nociceptive and depressive behaviour (Kim et al.; J Clin Invest. 2012, 122(8):2940-54).

Since proinflammatory cytokines have been implicated in the pathophysiology of both pain and depression, the regulation of brain IDO by proinflammatory cytokines serves as a critical mechanistic link in the comorbid relationship between pain and depression through the regulation of TRP metabolism.

Moreover, the KYN pathway has been associated with traumatic brain injury (TBI). TBI has been shown to induce a striking activation of the KYN pathway with sustained increase of QUIN (Yan et al.; Journal of Neuroinflammation 2015, 12 (110): 1-17). The exceeding production of QUIN together with increased IDO1 activation and mRNA expression in brain-injured areas suggests that TBI selectively induces a robust stimulation of the neurotoxic branch of the KYN pathway. QUIN's detrimental roles are supported by its association to adverse outcome potentially becoming an early prognostic factor post-TBI. Hence, IDO and/or TDO inhibitors may in addition be useful for the prevention/treatment of TBI.

Infection by bacteria, parasites, or viruses induces a strong IFN-γ-dependent inflammatory response. IDO can dampen protective host immunity, thus indirectly leading to increased pathogen burdens. For example, in mice infected with murine leukaemia virus (MuLV), IDO was found to be highly expressed, and ablation of IDO enhanced control of viral replication and increased survival (Hoshi et al.; J Immunol. 2010, 185(6):3305-3312). In a model of influenza infection, the immunosuppressive effects of IDO could predispose lungs to secondary bacterial infection (van der Sluijs et al.; J Infect Dis. 2006, 193(2): 214-22). Hence, IDO activity was increased in community-acquired pneumonia (CAP), and this activity was associated with the severity and outcome of this disease. These results suggest that IDO activity can predict prognosis of CAP (Suzuki et al.; J Infect. 2011 September; 63(3):215-22).

In Chagas Disease, which is caused by the Trypanosoma cruzi parasite, KYN is increased in patients and correlates with disease severity (Maranon et al.; Parasite Immunol. 2013, 35 (5-6):180-7). Infection with Chlamydia trachomatis induces the production of a large amount of IFN-γ which in turn causes IDO induction. A study has shown that IDO mediated depletion of the TRP pool causes Chlamydia to convert into a persistent form which is highly adapted to survive in hostile environments (Barth and Raghuraman; Crit Rev Microbiol. 2014, 40(4):360-8). In patients with chronic cutaneous leishmaniasis, high levels of IDO mRNA expression has been detected in infectious lesions and was associated with the accumulation of intralesional Treg cells. Leishmania major infection in mice induces IDO expression in local cutaneous lesions and draining lymph nodes. Genetic and pharmacological ablation of IDO resulted in improved control of L. major. Cerebral malaria can be a fatal manifestation of Plasmodium falciparum infection in humans. IDO activity is increased in the mouse brain during cerebral malaria and inhibition of IDO in a mouse model of malaria enhanced the function of anti-malarial T cells and slightly reduce the parasite load (Barth and Raghuraman; Crit Rev Microbiol. 2014, 40(4):360-8).

Measuring serum concentrations of KYN and TRP and assessed IDO activity in patients with pulmonary tuberculosis showed significant increases in Kyn concentrations and IDO activity and significant decreases in Trp concentrations compared to control subjects. Interestingly, among the pulmonary tuberculosis patients, nonsurvivors had significantly higher Kyn concentrations and significantly lower Trp concentrations, resulting in a significant increase in IDO activity over that in survivors. Most importantly, multivariate analysis showed that the IDO activity was a significant independent predictor of death in pulmonary tuberculosis (Suzuki et al.; Clin Vaccine Immunol. 2012, 19(3): 436-442).

Therefore, IDO inhibitors could be used to improve the outcomes of patients with a wide variety of infectious diseases and inflammatory conditions. Given the role of TDO in controlling systemic TRP levels, TDO inhibitors could also be used to improve the outcomes of patients with a wide variety of infectious diseases and inflammatory conditions.

Patients infected with HIV have chronically reduced levels of plasma TRP and increased levels of KYN, and increased IDO expression (Murray; Lancet Infect Dis. 2003, 3(10):644-52). In HIV patients the upregulation of IDO acts to suppress immune responses to HIV antigens contributing to the immune evasion of the virus. A characteristic feature during advanced HIV infection is the preferential depletion of Th17 cells from both the gastrointestinal tract and blood. Interestingly, the loss of Th17 cells in HIV infection is accompanied by a concomitant rise in the frequency of induced Treg cells and directly correlated with IDO activity. Treg cells may dampen efficient HIV specific cellular immune responses while the progressive depletion of Th17 cells may increase susceptibility to mucosal infections. Thus, sustained IDO activation may establish a favourable environment for HIV persistence and contribute to the immunodeficiency seen in HIV-infected individuals with progressive disease (Barth and Raghuraman; Crit Rev Microbiol. 2014, 40(4):360-8). HIV patients, particularly those with HIV-linked dementia (Kandanearatchi & Brew; FEBS J. 2012, 279(8):1366-74), often have significantly elevated KYN levels in CSF. These levels are directly related to the development of neurocognitive decline (HIV-associated neurocognitive disorder (HAND)) and often the presence of severe psychotic symptoms (Stone & Darlington; Trends Pharmacol Sci. 2013, 34(2):136-43). Therefore, IDO and/or TDO inhibitors may in addition be useful for the treatment of HIV (AIDS including its manifestations such as cachexia, dementia and diarrhea).

As with HIV infection, patients chronically infected with HCV present increased KYN to TRP ratios in blood compared to patients with resolved HCV infections and healthy individuals (Larrea et al.; J Virol. 2007, 81(7):3662-6). Furthermore, it has been suggested that expression of IDO correlated with the pathogenesis of the disease and the high expression of IDO in progressively cirrhotic livers of HCV-infected patients might contribute to the development of hepatocellular carcinoma (Asghar et al.; Exp Ther Med. 2015, 9(3):901-4). Hence, IDO and/or TDO inhibitors may be useful for the treatment of patients chronically infected with HCV.

IDO plays a role in regulating mucosal immunity to the intestinal microbiota. IDO has been shown to regulate commensal induced antibody production in the gut; IDO-deficient mice had elevated baseline levels of immunoglobulin A (IgA) and immunoglobulin G (IgG) in the serum and increased IgA in intestinal secretions. Due to elevated antibody production, IDO deficient mice were more resistant to intestinal colonization by the gram-negative enteric bacterial pathogen Citrobacter rodentium than WT mice. IDO-deficient mice also displayed enhanced resistance to the colitis caused by infection with C. rodentium (Harrington et al.; Infect Immunol. 2008, 76(7):3045-53).

Therefore, pharmacological targeting of IDO/TDO activity may represent a new approach to manipulating intestinal immunity and controlling the pathology caused by enteric pathogens including colitis (Harrington et al.; Infect Immunol. 2008, 76(7):3045-53).

Recent literature highlights a role for IDO in metabolic disorders (Laurans et al.; Nature Medicine https://doi.org/10.1038/s41591-018-0060-4 (2018); Natividad et al.; Cell Metabolism 2018, 28: 1-13). It was found that Ido1 knockout mice that were fed a high-fat diet gained less weight, had a lower fat mass, better glucose and insulin tolerance and less macrophage infiltration into fat tissue than wild-type mice did. Treatment with an IDO inhibitor, L-1-MT, concurrent with a high-fat diet had a similar effect on insulin and glucose tolerance to that in the knockout. The fact that antibiotic treatment prevented Ido1 knockout mice from gaining weight on a high-fat diet and co-housing of Ido1 knockout and wt mice had metabolic measurements similar to those of Ido1 knockout mice suggested that the microbiota from Ido1 knock-out mice is protective. Consistent with these hypotheses, Ido-1 knock-out mice had different intestinal microbiota composition. TRP can be metabolized either by IDO to produce KYN or by the gut microbiota to produce indole derivatives such as indole-3-acetic acid, a ligand for the AhR. Depletion of IDO increased the levels of indole-3-acetic acid in the faeces. Indole-3-acetic acid induced activation of the AhR in intestinal immune cells increases the production of IL-17 and IL-22. Reduced levels of IL-22 were accompanied with dysfunction of the gut barrier. These data support the importance of IDO in controlling KYN and indole-3-acetic acid-activating AhR balance. Consistent with the observations in mice, people with obesity or type 2 diabetes had higher levels of KYN in their plasma and faeces and lower levels of indole-3-acetic acid in their faeces (Laurans et al.; Nature Medicine https://doi.org/10.1038/s41591-018-0060-4 (2018). Increased KYN levels were also found in fecal samples of individuals with metabolic syndrome compared to healthy subjects in another study (Natividad et al.; Cell Metabolism 2018, 28: 1-13). Thus far it is unknown whether the alterations of AhR agonist production by the gut microbiota is the primary event in metabolic syndrome pathogenesis. However, the therapeutic effects of the correction of this defect by applying an AhR agonist shows its involvement in the pathogenesis ((Natividad et al.; Cell Metabolism 2018, 28: 1-13). Hence IDO inhibitors through altering the balance of TRP derived AhR agonist balance could be useful in regulating metabolic disorders such as obesity, type 2 diabetes and/or fatty acid liver disease.

A cataract is a clouding of the lens inside the eye that leads to a decrease in vision. Recent studies suggest that KYNs might chemically alter protein structure in the human lens leading to cataract formation. In the human lens IDO activity is present mainly in the anterior epithelium (Takikawa et al.; Adv Exp Med Biol. 1999, 467: 241-5). Several KYNs, such as KYN, 3-HK, and 3-hydroxykynurenine glucoside (3-HK-G) have been detected in the lens; where they were thought to protect the retina by absorbing UV light and therefore are commonly referred to as UV filters. However, several recent studies show that KYNs are prone to deamination and oxidation to form α,β-unsaturated ketones that chemically react and modify lens proteins (Taylor et al.; Exp Eye Res. 2002; 75(2): 165-75). KYN mediated modification could contribute to the lens protein modifications during aging and cataractogenesis. They may also reduce the chaperone function of a-crystallin, which is necessary for maintaining lens transparency.

Transgenic mouse lines that overexpress human IDO in the lens developed bilateral cataracts within 3 months of birth. It was demonstrated that IDO-mediated production of KYNs results in defects in fibre cell differentiation and their apoptosis (Mailankot et al.; Lab Invest. 2009; 89(5):498-512). Therefore, inhibition of IDO/TDO may slow the progression of cataract formation.

Endometriosis, the presence of endometrium outside the uterine cavity, is a common gynaecological disorder, causing abdominal pain, dyspareunia and infertility. IDO expression was found to be higher in eutopic endometrium from women with endometriosis by microarray analysis (Burney et al.; Endocrinology. 2007; 148(8): 3814-26; Aghajanova et al.; Reprod Sci. 2011, 18(3):229-251). Furthermore, IDO was shown to enhance the survival and invasiveness of endometrial stromal cells (Mei et al.; Int J Clin Exp Pathol. 2013; 6(3): 431-44). Therefore, an IDO/TDO inhibitor may be used as a treatment for endometriosis.

The process of implantation of an embryo requires mechanisms that prevent allograft rejection; and tolerance to the fetal allograft represents an important mechanism for maintaining a pregnancy. Cells expressing IDO in the foeto-maternal interface protect the allogeneic foetus from lethal rejection by maternal immune responses. Inhibition of IDO by exposure of pregnant mice to 1-methyl-tryptophan induced a T cell-mediated rejection of allogeneic concepti, whereas syngeneic concepti were not affected; this suggests that IDO expression at the foetal-maternal interface is necessary to prevent rejection of the foetal allograft (Munn et al.; Science 1998, 281(5380): 1191-3). Accumulating evidence indicates that IDO production and normal function at the foetal-maternal interface may play a prominent role in pregnancy tolerance (Duff and Kindler; J Leukoc Biol. 2013, 93(5): 681-700). Therefore, an IDO/TDO inhibitor could be used as a contraceptive or abortive agent.

In experimental chronic renal failure, activation of IDO leads to increased blood levels of KYNs (Tankiewicz et al.; Adv Exp Med Biol. 2003, 527:409-14), and in uremic patients KYN-modified proteins are present in urine (Sala et al.; J Biol Chem. 2004, 279(49):51033-41). Further, renal IDO expression may be deleterious during inflammation, because it enhances tubular cell injury.

In coronary heart disease, inflammation and immune activation are associated with increased blood levels of KYN (Wirleitner et al.; Eur J Clin Invest. 2003, 33(7):550-4) possibly via interferon-y-mediated activation of IDO.

Cardiac surgery involving extra-corporeal circulation can lead to cognitive dysfunction. As such surgery is associated with signs of inflammation and pro-inflammatory mediators activate tryptophan oxidation to neuroactive kynurenines which modulate NMDA receptor function and oxidative stress. Post anaesthesia cognitive dysfunction has often been correlated with these sequelae. Recently these deficits have been shown to be correlated with changes in KYN pathway markers, but not cytokines, following cardiac surgery and in recovering stroke patients (Forrest et al.; J. Neurochem. 201, 119(1):136-52).

In general, TRP catabolism has been reported to be altered in stroke. The activation of the KYN pathway in the acute phase of stroke may participate in the ischemic damage by direct mechanisms which include excitotoxicity and oxidative stress among others, since inhibition of the KYN pathway decreases brain injury in animal models of stroke. Probably, an interplay between the immune system and the KYN pathway could exist after stroke, but also different inflammatory-independent mechanisms could mediate a role in the regulation of this pathway, modulating the rate-limiting enzymes of TRP catabolism. Interestingly, the KYN pathway after cerebral ischemia could also play a role during the chronic phase of this pathology in which stroke survivors present a high incidence of disabilities such as dementia and depression or even being a risk factor for stroke outcome and mortality. All together the KYN and TRP catabolism could have a significant role in after cerebral ischemia and IDO/TDO inhibitors may provide new pharmacological tools in both acute and chronic phases of stroke (Cuartero et al.; Curr Pharm Des. 2016; 22(8): 1060-1073).

The present invention provides novel compounds of Formula (I) which inhibit the activity of IDO and/or TDO enzymes.

1) A first embodiment of the present invention relates to compounds of Formula (I)

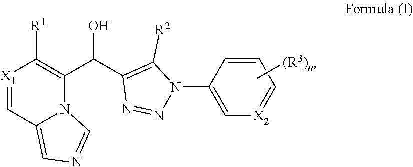

wherein

X₁ represents nitrogen or carbon (especially carbon);

X₂ represents nitrogen or carbon (especially carbon);

R¹ represents
C₁₋₄-alkyl (especially methyl or ethyl);
C₃₋₅-cycloalkyl (especially cyclopropyl); or
halogen (especially chlorine);

R² represents
hydrogen;
C₁₋₃-alkyl (especially methyl or ethyl); or
halogen (especially chlorine);

each R³ independently represents
C₁₋₄-alkyl (especially methyl);
C₁₋₃-alkoxy-C₁₋₄-alkyl (especially methoxymethyl);
halogen (especially fluorine, chlorine or bromine);
—OR⁴, wherein R⁴ represents hydrogen, C₁₋₄-alkyl (especially methyl or ethyl), hydroxy-C₂₋₅-alkyl (especially 2-hydroxy-2-methylpropyl or 3-hydroxy-3-methylbutyl), (oxetan-3-yl)-C₁₋₃-alkyl (especially (oxetan-3-yl)-methyl) or (3-fluoro-oxetan-3-yl)-C₁₋₃-alkyl (especially (3-fluoro-oxetan-3-yl)-methyl);
—NR^{N1}R^{N2}, wherein
   R^{N1} represents hydrogen and R^{N2} represents —(C═O)—R^{CO}, wherein R^{CO} represents C₁₋₃-alkoxy (especially methoxy);
   R^{N1} and R^{N2} independently represent hydrogen or C₁₋₃-alkyl (especially methyl);
   R^{N1} and R^{N2}, together with the nitrogen atom to which they are attached, form a 4- to 6-membered saturated heterocyclic ring comprising one nitrogen ring atom (notably azetidinyl, pyrrolidinyl or piperidinyl; especially pyrrolidinyl); or
   R^{N1} represents C₁₋₃-alkyl (especially methyl) and R^{N2} represents 1,2-ethanediyl such that the fragment

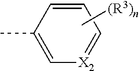

of Formula (I) represents 1-(C₁₋₃-alkyl)-2,3-dihydro-indol-5-yl (especially 1-methyl-2,3-dihydro-indol-5-yl);
2-oxa-6-aza-spiro[3.3]hept-6-yl or 6-oxa-1-aza-spiro[3.3]hept-1-yl; and
n represents 0, 1, 2, 3, 4 or 5 (especially 0, 1, 2 or 3) (i.e. (R³)ₙ represents 0, or 1 to 5 substituents R³, wherein it is understood that when n=0, R³ is non-existent).

[In a sub-embodiment of embodiment 1), one substituent R³ (especially —OR⁴ or —NR^{N1}R^{N2}) is attached in para-position with regard to the point of attachment to the rest of the molecule, and no further R³ is present, or the remaining R³, if present, is/are especially selected from halogen (especially fluorine, chlorine or bromine)]

2) Another embodiment of the present invention relates to compounds according to embodiment 1), wherein

X₁ represents nitrogen or carbon (especially carbon);

X₂ represents nitrogen or carbon (especially carbon);

R¹ represents
C₁₋₄-alkyl (especially methyl or ethyl);
C₃₋₅-cycloalkyl (especially cyclopropyl); or
halogen (especially chlorine);

R² represents
hydrogen; or
C₁₋₃-alkyl (especially methyl);

each R³ independently represents
C₁₋₄-alkyl (especially methyl);
C₁₋₃-alkoxy-C₁₋₄-alkyl (especially methoxymethyl);
halogen (especially fluorine, chlorine or bromine);
—OR⁴, wherein R⁴ represents hydrogen, C₁₋₄-alkyl (especially methyl or ethyl), hydroxy-C₂₋₅-alkyl (especially 2-hydroxy-2-methylpropyl), (oxetan-3-yl)-C₁₋₃-alkyl (especially (oxetan-3-yl)-methyl) or (3-fluoro-oxetan-3-yl)-C₁₋₃-alkyl (especially (3-fluoro-oxetan-3-yl)-methyl); or
—NR^{N1}R^{N2}, wherein R^{N1} represents hydrogen and R^{N2} represents —(C═O)—R^{CO}, wherein R^{CO} represents C₁₋₃-alkoxy (especially methoxy); and
n represents 0, 1, 2, 3, 4 or 5 (especially 0, 1, 2 or 3) (i.e. (R³)ₙ represents 0, or 1 to 5 substituents R³, wherein it is understood that when n=0, R³ is non-existent).

[In a sub-embodiment of embodiment 2), one substituent R³ (especially —OR⁴ or —NR^{N1}R^{N2}) is attached in para-position with regard to the point of attachment to the rest of the molecule, and no further R³ is present, or the remaining R³, if present, is/are especially selected from halogen (especially fluorine, chlorine or bromine)]

Definitions provided hereinbelow are intended to apply uniformly to the compounds of Formula (I)/(II), as defined in any one of embodiments 1) to 20), and, mutatis mutandis, throughout the description and the claims unless an otherwise expressly set out definition provides a broader or narrower definition. It is well understood that a definition or preferred definition of a term defines and may replace the respective term independently of (and in combination with) any definition or preferred definition of any or all other terms as defined herein. If not explicitly defined otherwise in the respective embodiment or claim, groups defined herein are unsubstituted.

The term “alkyl”, used alone or in combination, refers to a saturated straight or branched hydrocarbon chain containing one to six carbon atoms. Examples are methyl, ethyl, n-propyl, iso-propyl, n-butyl, tert-butyl, sec-butyl, iso-butyl, n-pentyl, 1,1-dimethylpropyl, 2,2-dimethylpropyl, 3-methylbutyl, 3-pentyl, 2-pentyl, 1,2-dimethylpropyl and 2-methylbutyl. The term “C_{x-y}-alkyl” (x and y each being an integer), used alone or in combination, refers to a saturated straight or branched hydrocarbon chain with x to y carbon atoms. Thus, the term C₁₋₄-alkyl, alone or in combination with other groups, means saturated, branched or straight chain groups with one to four carbon atoms. Examples of C₁₋₄-alkyl groups are methyl, ethyl, n-propyl, iso-propyl, n-butyl, tert-butyl, sec-butyl and iso-butyl.

The term “cycloalkyl”, used alone or in combination, refers to a saturated monocyclic hydrocarbon ring containing three to six carbon atoms. The term “C_{x-y}-cycloalkyl” (x and y each being an integer), refers to a saturated monocyclic hydrocarbon ring containing x to y carbon atoms. Examples of C₃₋₅-cycloalkyl group are cyclopropyl, cyclobutyl, and cyclopentyl; especially cyclopropyl and cyclobutyl; notably cyclopropyl. All of the above groups are unsubstituted or substituted as explicitly defined.

The term “alkoxy”, used alone or in combination, refers to an alkyl-O— group wherein the alkyl group is as defined before. The term “C_{x-y}-alkoxy” (x and y each being an integer) refers to an alkoxy group as defined before containing x to y carbon atoms. For example, the term “C_{x-y}-alkoxy” (x and y each being an integer), used alone or in combination, refers to an alkyl-O— group wherein the alkyl group refers to a straight or branched hydrocarbon chain with x to y carbon atoms. For example, a C₁₋₃-alkoxy refers to methoxy, ethoxy, n-propoxy and iso-propoxy; especially methoxy.

The term “halogen” means fluorine, chlorine, bromine or iodine; especially fluorine, chlorine or bromine. For halogen substituents attached to phenyl, pyridinyl, imidazo[1,5-a]pyridinyl, or imidazo[1,5-a]pyrazinyl independently preferred are fluorine and chlorine.

The term “hydroxyalkyl”, used alone or in combination, refers to an alkyl group as defined before, wherein one hydrogen atom has been replaced by a hydroxy group. Representative examples of hydroxyalkyl groups include 2-hydroxyethyl, 2-hydroxypropyl, 3-hydroxypropyl, 4-hydroxybutyl, 2-hydroxybutyl, 3-hydroxybutyl, 3-hydroxy-1-methylpropyl, 3-hydroxy-2-methylpropyl, 2-hydroxy-1-methylpropyl 2-hydroxy-2-methylpropyl, 3-hydroxy-1,1-dimethylpropyl, 3-hydroxy-2,2-dimethylpropyl, 3-hydroxy-1,2-dimethylpropyl, 3-hydroxy-1-ethylpropyl, 1-hydroxymethyl-butyl, 2-hydroxypentyl, 3-hydroxypentyl, 4-hydroxypentyl, 5-hydroxypentyl, 2-hydroxy-3-methylbutyl and 3-hydroxy-3-methylbutyl. The term “hydroxy-C_{x-y}-alkyl” (x and y each being an integer), used alone or in combination, refers to a hydroxyalkyl group as defined before wherein the alkyl group contains x to y carbon atoms. A hydroxy-C₂₋₅-alkyl group is a hydroxyalkyl group as defined before which contains from two to five carbon atoms, especially 3-hydroxypropyl, 2-hydroxy-2-methylpropyl, 2-hydroxyethyl, 3-hydroxy-2,2-dimethylpropyl or 3-hydroxy-3-methylbutyl.

n represents the number of R³ substituents in the phenyl or pyridinyl ring as depicted in Formula (I)/(II), wherein n is an integer selected from a group consisting of 0, 1, 2, 3, 4 and 5; especially 1, 2, 3, 4 and 5; notably 2, 3 and 4. It is understood that when n=0, no substituent R³ is present in Formula (I)/(II).

It is understood that when X₂ represents a carbon, X₂ represents a CH or a C—R³ group. It is further understood that when X₁ represents a carbon, X₁ represents a CH group.

The term “C₁₋₃-alkoxy-C₁₋₄-alkyl” refers to an alkyl group as defined before, wherein one of its hydrogen atoms has been replaced by a C₁₋₃-alkoxy group as defined before. Representative examples of C₁₋₃-alkoxy-C₁₋₄-alkyl include methoxymethyl, ethoxymethyl, propoxyethyl, ethoxyethyl, ethoxypropyl and propoxypropyl. A preferred example of C₁₋₃-alkoxy-C₁₋₄-alkyl is methoxymethyl.

The term “oxetan-3-yl-C₁₋₃-alkyl” refers to an alkyl group as defined before, wherein one of its hydrogen atoms has been replaced by an oxetane ring, wherein said oxetane ring is attached to said alkyl group in ring position 3. Representative examples include oxetan-3-yl-methyl, 1-(oxetan-3-yl)-ethyl, 2-(oxetan-3-yl)-ethyl and 1-(oxetan-3-yl)-propyl; especially oxetan-3-yl-methyl.

The term “(3-fluoro-oxetan-3-yl)-C₁₋₃-alkyl” refers to an oxetan-3-yl-C₁₋₃-alkyl group as defined before, wherein the hydrogen atom in position 3 of the oxetane ring has been replaced by fluorine. Representative examples include (3-fluoro-oxetan-3-yl)-methyl, 2-(3-fluoro-oxetan-3-yl)-ethyl and 3-(3-fluoro-oxetan-3-yl)-propyl; especially (3-fluoro-oxetan-3-yl)-methyl.

3) A further embodiment relates to compounds according to any one of embodiments 1) or 2), wherein X₁ represents carbon.

4) A further embodiment relates to compounds according to any one of embodiments 1) or 2), wherein X₁ represents nitrogen.

5) A further embodiment relates to compounds according to any one of embodiments 1) to 4), wherein X₂ represents carbon.

6) A further embodiment relates to compounds according to any one of embodiments 1) to 4), wherein X₂ represents nitrogen.

7) A further embodiment relates to compounds according to any one of embodiments 1) to 6), wherein R¹ represents C₃₋₅-cycloalkyl (especially cyclopropyl) or halogen (especially chlorine); notably R¹ represents cyclopropyl.

8) A further embodiment relates to compounds according to any one of embodiments 1) to 6), wherein R¹ represents C₁₋₄-alkyl; notably R¹ represents ethyl.

9) A further embodiment relates to compounds according to any one of embodiments 1) to 8), wherein R² represents hydrogen.

10) A further embodiment relates to compounds according to any one of embodiments 1) to 8), wherein R² represents hydrogen or C₁₋₃-alkyl (especially methyl or ethyl).

11) A further embodiment relates to compounds according to any one of embodiments 1) to 10), wherein R³ independently represents
C₁₋₃-alkoxy-C₁₋₄-alkyl (especially methoxymethyl);
halogen (especially fluorine, chlorine or bromine);
—OR⁴, wherein R⁴ represents hydrogen, C₁₋₄-alkyl (especially methyl or ethyl), hydroxy-C₂₋₅-alkyl (especially 2-hydroxy-2-methylpropyl or 3-hydroxy-3-methylbutyl), (oxetan-3-yl)-C₁₋₃-alkyl (especially (oxetan-3-yl)-methyl) or (3-fluoro-oxetan-3-yl)-C₁₋₃-alkyl (especially (3-fluoro-oxetan-3-yl)-methyl);
—NR^{N1}R^{N2}, wherein R^{N1} represents hydrogen and R^{N2} represents —(C═O)—R^{CO}, wherein R^{CO} represents C₁₋₃-alkoxy (especially methoxy).

12) A further embodiment relates to compounds according to any one of embodiments 1) to 10), wherein

R³ independently represents
C₁₋₃-alkoxy-C₁₋₄-alkyl (especially methoxymethyl);
halogen (especially fluorine, chlorine or bromine);
—OR⁴, wherein R⁴ represents hydrogen, C₁₋₄-alkyl (especially methyl or ethyl), hydroxy-C₂₋₅-alkyl (especially 2-hydroxy-2-methylpropyl or 3-hydroxy-3-methylbutyl), (oxetan-3-yl)-C₁₋₃-alkyl (especially (oxetan-3-yl)-methyl) or (3-fluoro-oxetan-3-yl)-C₁₋₃-alkyl (especially (3-fluoro-oxetan-3-yl)-methyl).

13) A further embodiment relates to compounds according to any one of embodiments 1) to 12), wherein n represents 1, 2 or 3 (especially 2 or 3).

14) A further embodiment relates to compounds according to any one of embodiments 1) and 3) to 10),

wherein

n represents 1, 2 or 3;

one substituent R³ represents
—OR⁴, wherein R⁴ represents hydrogen, C₁₋₄-alkyl (especially methyl or ethyl), hydroxy-C₂₋₅-alkyl (especially 2-hydroxy-2-methylpropyl or 3-hydroxy-3-methylbutyl), (oxetan-3-yl)-C₁₋₃-alkyl (especially (oxetan-3-yl)-methyl) or (3-fluoro-oxetan-3-yl)-C₁₋₃-alkyl (especially (3-fluoro-oxetan-3-yl)-methyl); or
—NR^{N1}R^{N2}, wherein
   R^{N1} represents hydrogen and R^{N2} represents —(C═O)—R^{CO}, wherein R^{CO} represents C₁₋₃-alkoxy (especially methoxy);
   R^{N1} and R^{N2} independently represent hydrogen or C₁₋₃-alkyl (especially methyl);
   R^{N1} and R^{N2}, together with the nitrogen atom to which they are attached, form a 4- to 6-membered saturated heterocyclic ring comprising one nitrogen ring atom (notably azetidinyl, pyrrolidinyl or piperidinyl; especially pyrrolidinyl); or
2-oxa-6-aza-spiro[3.3]hept-6-yl or 6-oxa-1-aza-spiro[3.3]hept-1-yl;

wherein said one substituent is attached in para-position with regard to the point of attachment to the rest of the molecule and the remaining R³, if present, is/are selected from halogen (especially fluorine or chlorine).

15) A further embodiment relates to compounds according to any one of embodiments 1) to 10), wherein

n represents 1, 2 or 3;

one substituent R³ represents
—OR⁴, wherein R⁴ represents hydrogen, C₁₋₄-alkyl (especially methyl or ethyl), hydroxy-C₂₋₅-alkyl (especially 2-hydroxy-2-methylpropyl or 3-hydroxy-3-methylbutyl), (oxetan-3-yl)-C₁₋₃-alkyl (especially (oxetan-3-yl)-methyl) or (3-fluoro-oxetan-3-yl)-C₁₋₃-alkyl (especially (3-fluoro-oxetan-3-yl)-methyl); or
—NR^{N1}R^{N2}, wherein R^{N1} represents hydrogen and R^{N2} represents —(C═O)—R^{CO}, wherein R^{CO} represents C₁₋₃-alkoxy (especially methoxy);

wherein said one substituent is attached in para-position with regard to the point of attachment to the rest of the molecule and the remaining R³, if present, is/are selected from halogen (especially fluorine or chlorine).

16) A further embodiment relates to compounds according to any one of embodiments 1) to 10), wherein

n represents 1, 2 or 3;

one substituent R³ represents
—OR⁴, wherein R⁴ represents hydrogen, C₁₋₄-alkyl (especially methyl or ethyl), hydroxy-C₂₋₅-alkyl (especially 2-hydroxy-2-methylpropyl or 3-hydroxy-3-methylbutyl), (oxetan-3-yl)-C₁₋₃-alkyl (especially (oxetan-3-yl)-methyl) or (3-fluoro-oxetan-3-yl)-C₁₋₃-alkyl (especially (3-fluoro-oxetan-3-yl)-methyl);

wherein said one substituent is attached in para-position with regard to the point of attachment to the rest of the molecule and the remaining R³, if present, is/are selected from halogen (especially fluorine or chlorine).

17) A further embodiment relates to compounds according to any one of embodiments 1) to 10), wherein the fragment

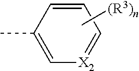

of Formula (I) represents
phenyl, 4-hydroxyphenyl, 4-methoxyphenyl, 3-bromo-4-methoxyphenyl, 4-methylphenyl, 3-chloro-4-hydroxyphenyl, 3-chloro-4-methoxyphenyl, 3-fluoro-4-hydroxyphenyl, 3-fluoro-4-methoxyphenyl, 2-fluoro-3-chloro-4-methoxyphenyl, 3-chloro-4-methoxy-5-fluorophenyl, 2-fluoro-4-methoxy-5-chlorophenyl, 2,5-difluoro-4-methoxyphenyl, 4-((oxetan-3-yl)methoxy)-phenyl, 3-fluoro-4-((oxetan-3-yl)methoxy)-phenyl, 4-((3-fluoro-oxetan-3-yl)methoxy)-phenyl, 3-fluoro-4-((3-fluoro-oxetan-3-yl)methoxy)-phenyl, 4-(methoxy-carboxamido)-phenyl, 4-(2-hydroxy-2-methylpropoxy)-phenyl, 4-(methoxymethyl)-phenyl; or 4-ethoxypyridin-3-yl; or, in addition to the above-listed, 3-fluoro-4-(2-hydroxy-2-methylpropoxy)-phenyl, or 6-ethoxypyridin-3-yl.

18) A further embodiment relates to compounds according to any one of embodiments 1) and 3) to 10), wherein the fragment

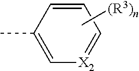

of Formula (I) represents
phenyl, 4-hydroxyphenyl, 4-methoxyphenyl, 3-bromo-4-methoxyphenyl, 4-methylphenyl, 3-chloro-4-hydroxyphenyl, 3-chloro-4-methoxyphenyl, 3-fluoro-4-hydroxyphenyl, 3-fluoro-4-methoxyphenyl, 2-fluoro-3-chloro-4-methoxyphenyl, 3-chloro-4-methoxy-5-fluorophenyl, 2-fluoro-4-methoxy-5-chlorophenyl, 2,5-difluoro-4-methoxyphenyl, 4-((oxetan-3-yl)methoxy)-phenyl, 3-fluoro-4-((oxetan-3-yl)methoxy)-phenyl, 4-((3-fluoro-oxetan-3-yl)methoxy)-phenyl, 3-fluoro-4-((3-fluoro-oxetan-3-yl)methoxy)-phenyl, 4-(methoxy-carboxamido)-phenyl, 4-(2-hydroxy-2-methylpropoxy)-phenyl, 4-(methoxymethyl)-phenyl; or 4-ethoxypyridin-3-yl; or, in addition to the above-listed, 3-fluoro-4-(2-hydroxy-2-methylpropoxy)-phenyl, or 6-ethoxypyridin-3-yl; or
3-fluoro-4-(3-hydroxy-3-methylbutoxy)-phenyl, 3-chloro-4-(3-hydroxy-3-methylbutoxy)-phenyl, 2,5-difluoro-4-((3-fluoro-oxetan-3-yl)methoxy)-phenyl, 2,5-difluoro-4-((oxetan-3-yl)methoxy)-phenyl, 2,5-difluoro-4-(2-hydroxy-2-methylpropoxy)-phenyl, 4-(2-oxa-6-aza-spiro[3.3]hept-6-yl)-phenyl, 4-(6-oxa-1-aza-spiro[3.3]hept-1-yl)-phenyl, 1-methyl-2,3-dihydro-1H-indol-5-yl, 4-amino-phenyl, 4-(methylamino)-phenyl, 4-(pyrrolidin-1-yl)-phenyl, 4-dimethylamino-phenyl, 2-fluoro-phenyl, or 2,4-difluoro-phenyl.

19) A further embodiment relates to compounds according to any one of embodiments 1) to 2), wherein

X₁ represents carbon; R¹ represents methyl, ethyl, cyclopropyl or chlorine; R² represents hydrogen; and wherein the fragment

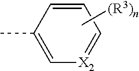

of Formula (I) represents 4-hydroxyphenyl, 4-methoxyphenyl, 3-chloro-4-hydroxyphenyl, 3-chloro-4-methoxyphenyl, 3-fluoro-4-hydroxyphenyl, 3-fluoro-4-methoxyphenyl, 2-fluoro-4-methoxy-5-chlorophenyl, 2,5-difluoro-4-methoxyphenyl, 3-fluoro-4-((oxetan-3-yl)methoxy)-phenyl, 4-((3-fluoro-oxetan-3-yl)methoxy)-phenyl, 4-(methoxy-carboxamido)-phenyl, or 4-(2-hydroxy-2-methylpropoxy)-phenyl; or 4-ethoxypyridin-3-yl.

20) Another embodiment relates to compounds according to any one of embodiments 1) to 19), which are also compounds of Formula (II) (i.e. wherein the asymmetric carbon atom bearing the OH group, to which the fragment [1,2,3]triazol-1,4-diyl is attached has the absolute configuration depicted in Formula (II) (i.e. said asymmetric carbon atom is in absolute (R)-configuration)).

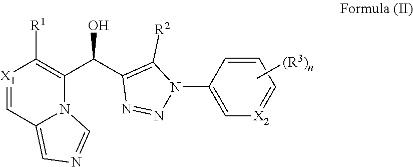

21) Another embodiment relates to a compound according to any one of embodiments 1) or 2) selected from a group consisting of:
(6-Chloro-imidazo[1,5-a]pyridin-5-yl)-(1-phenyl-1H-[1,2,3]triazol-4-yl)-methanol;
(R)-(6-Chloro-imidazo[1,5-a]pyridin-5-yl)-(1-phenyl-1H-[1,2,3]triazol-4-yl)-methanol;
(6-Methyl-imidazo[1,5-a]pyridin-5-yl)-(1-phenyl-1H-[1,2,3]triazol-4-yl)-methanol;
(R)-(6-Methyl-imidazo[1,5-a]pyridin-5-yl)-(1-phenyl-1H-[1,2,3]triazol-4-yl)-methanol;
(6-Cyclopropyl-imidazo[1,5-a]pyrazin-5-yl)-(1-phenyl-1H-[1,2,3]triazol-4-yl)-methanol;
(R)-(6-Cyclopropyl-imidazo[1,5-a]pyrazin-5-yl)-(1-phenyl-1H-[1,2,3]triazol-4-yl)-methanol;
(6-Cyclopropyl-imidazo[1,5-a]pyridin-5-yl)-[1-(4-methoxy-phenyl)-1H-[1,2,3]triazol-4-yl]-methanol;
(6-Cyclopropyl-imidazo[1,5-a]pyridin-5-yl)-(1-p-tolyl-1H-[1,2,3]triazol-4-yl)-methanol;
(4-{4-[(6-Cyclopropyl-imidazo[1,5-a]pyridin-5-yl)-hydroxy-methyl]-[1,2,3]triazol-1-yl}-phenyl)-carbamic acid methyl ester;
2-Chloro-4-{4-[(6-cyclopropyl-imidazo[1,5-a]pyridin-5-yl)-hydroxy-methyl]-[1,2,3]triazol-1-yl}-phenol;
2-Chloro-4-{4-[(R)-(6-cyclopropyl-imidazo[1,5-a]pyridin-5-yl)-hydroxy-methyl]-[1,2,3]triazol-1-yl}-phenol;
2-Chloro-4-{4-[(S)-(6-cyclopropyl-imidazo[1,5-a]pyridin-5-yl)-hydroxy-methyl]-[1,2,3]triazol-1-yl}-phenol;
[1-(3-Chloro-4-methoxy-phenyl)-1H-[1,2,3]triazol-4-yl]-(6-cyclopropyl-imidazo[1,5-a]pyridin-5-yl)-methanol;
(R)-[1-(3-Chloro-4-methoxy-phenyl)-1H-[1,2,3]triazol-4-yl]-(6-cyclopropyl-imidazo[1,5-a]pyridin-5-yl)-methanol;
(6-Cyclopropyl-imidazo[1,5-a]pyridin-5-yl)-[1-(6-ethoxy-pyridin-3-yl)-1H-[1,2,3]triazol-4-yl]-methanol;
2-Chloro-4-{4-[(6-cyclopropyl-imidazo[1,5-a]pyrazin-5-yl)-hydroxy-methyl]-[1,2,3]triazol-1-yl}-phenol;
2-Chloro-4-{4-[(R)-(6-cyclopropyl-imidazo[1,5-a]pyrazin-5-yl)-hydroxy-methyl]-[1,2,3]triazol-1-yl}-phenol;
[1-(3-Chloro-4-methoxy-phenyl)-1H-[1,2,3]triazol-4-yl]-(6-cyclopropyl-imidazo[1,5-a]pyrazin-5-yl)-methanol;
(R)-[1-(3-Chloro-4-methoxy-phenyl)-1H-[1,2,3]triazol-4-yl]-(6-cyclopropyl-imidazo[1,5-a]pyrazin-5-yl)-methanol;
(4-{4-[(6-Cyclopropyl-imidazo[1,5-a]pyrazin-5-yl)-hydroxy-methyl]-[1,2,3]triazol-1-yl}-phenyl)-carbamic acid methyl ester;
(6-Cyclopropyl-imidazo[1,5-a]pyrazin-5-yl)-[1-(6-ethoxy-pyridin-3-yl)-1H-[1,2,3]triazol-4-yl]-methanol;
4-{4-[(6-Cyclopropyl-imidazo[1,5-a]pyridin-5-yl)-hydroxy-methyl]-[1,2,3]triazol-1-yl}-phenol;
4-{4-[(6-Cyclopropyl-imidazo[1,5-a]pyrazin-5-yl)-hydroxy-methyl]-[1,2,3]triazol-1-yl}-phenol;
(6-Cyclopropyl-imidazo[1,5-a]pyrazin-5-yl)-{1-[4-(3-fluoro-oxetan-3-ylmethoxy)-phenyl]-1H-[1,2,3]triazol-4-yl}-methanol;
(6-Cyclopropyl-imidazo[1,5-a]pyrazin-5-yl)-[1-(4-methoxy-phenyl)-1H-[1,2,3]triazol-4-yl]-methanol;
1-(4-{4-[(6-Cyclopropyl-imidazo[1,5-a]pyrazin-5-yl)-hydroxy-methyl]-[1,2,3]triazol-1-yl}-phenoxy)-2-methyl-propan-2-ol;
(6-Cyclopropyl-imidazo[1,5-a]pyridin-5-yl)-{1-[4-(3-fluoro-oxetan-3-ylmethoxy)-phenyl]-1H-[1,2,3]triazol-4-yl}-methanol;
(6-Chloro-imidazo[1,5-a]pyridin-5-yl)-[1-(4-methoxy-phenyl)-5-methyl-1H-[1,2,3]triazol-4-yl]-methanol;
(R)-(6-Cyclopropyl-imidazo[1,5-a]pyrazin-5-yl)-[1-(3-fluoro-4-methoxy-phenyl)-1H-[1,2,3]triazol-4-yl]-methanol;
(R)-(6-Cyclopropyl-imidazo[1,5-a]pyrazin-5-yl)-[1-(2,5-difluoro-4-methoxy-phenyl)-1H-[1,2,3]triazol-4-yl]-methanol;
(R)-[1-(3-Bromo-4-methoxy-phenyl)-1H-[1,2,3]triazol-4-yl]-(6-cyclopropyl-imidazo[1,5-a]pyrazin-5-yl)-methanol;
(R)-(6-Cyclopropyl-imidazo[1,5-a]pyrazin-5-yl)-[1-(4-methoxymethyl-phenyl)-1H-[1,2,3]triazol-4-yl]-methanol;
(R)-[1-(5-Chloro-2-fluoro-4-methoxy-phenyl)-1H-[1,2,3]triazol-4-yl]-(6-cyclopropyl-imidazo[1,5-a]pyrazin-5-yl)-methanol;
(R)-[1-(3-Chloro-5-fluoro-4-methoxy-phenyl)-1H-[1,2,3]triazol-4-yl]-(6-cyclopropyl-imidazo[1,5-a]pyrazin-5-yl)-methanol;
4-{4-[(R)-(6-Cyclopropyl-imidazo[1,5-a]pyrazin-5-yl)-hydroxy-methyl]-[1,2,3]triazol-1-yl}-2-fluoro-phenol;
(R)-(6-Cyclopropyl-imidazo[1,5-a]pyrazin-5-yl)-{1-[3-fluoro-4-(3-fluoro-oxetan-3-ylmethoxy)-phenyl]-1H-[1,2,3]triazol-4-yl}-methanol;
1-(4-{4-[(R)-(6-Cyclopropyl-imidazo[1,5-a]pyrazin-5-yl)-hydroxy-methyl]-[1,2,3]triazol-1-yl}-2-fluoro-phenoxy)-2-methyl-propan-2-ol;
(R)-(6-Chloro-imidazo[1,5-a]pyridin-5-yl)-[1-(4-methoxy-phenyl)-1H-[1,2,3]triazol-4-yl]-methanol;
(R)-(6-Chloro-imidazo[1,5-a]pyridin-5-yl)-[1-(2,5-difluoro-4-methoxy-phenyl)-1H-[1,2,3]triazol-4-yl]-methanol;
[1-(2,5-Difluoro-4-methoxy-phenyl)-1H-[1,2,3]triazol-4-yl]-(6-ethyl-imidazo[1,5-a]pyrazin-5-yl)-methanol;
(R)-[1-(3-Chloro-2-fluoro-4-methoxy-phenyl)-1H-[1,2,3]triazol-4-yl]-(6-cyclopropyl-imidazo[1,5-a]pyrazin-5-yl)-methanol;
(6-Ethyl-imidazo[1,5-a]pyridin-5-yl)-[1-(4-methoxy-phenyl)-1H-[1,2,3]triazol-4-yl]-methanol;
(R)-(6-Ethyl-imidazo[1,5-a]pyridin-5-yl)-[1-(4-methoxy-phenyl)-1H-[1,2,3]triazol-4-yl]-methanol;
[1-(2,5-Difluoro-4-methoxy-phenyl)-1H-[1,2,3]triazol-4-yl]-(6-ethyl-imidazo[1,5-a]pyridin-5-yl)-methanol;
(R)-[1-(2,5-Difluoro-4-methoxy-phenyl)-1H-[1,2,3]triazol-4-yl]-(6-ethyl-imidazo[1,5-a]pyridin-5-yl)-methanol; and
(R)-(6-Cyclopropyl-imidazo[1,5-a]pyrazin-5-yl)-{1-[3-fluoro-4-(oxetan-3-ylmethoxy)-phenyl]-1H-[1,2,3]triazol-4-yl}-methanol.

It is understood that all compounds listed in embodiment 21) are notably in enriched (R)-stereoisomeric form, and especially in essentially pure (R)-stereoisomeric form.

22) Another embodiment relates to a compound according to embodiment 1) selected from a group consisting of:
(6-Chloro-imidazo[1,5-a]pyridin-5-yl)-[5-ethyl-1-(4-methoxy-phenyl)-1H-[1,2,3]triazol-4-yl]-methanol;
(6-Chloro-imidazo[1,5-a]pyridin-5-yl)-(5-methyl-1-phenyl-1H-[1,2,3]triazol-4-yl)-methanol;
[1-(5-Chloro-2-fluoro-4-methoxy-phenyl)-5-methyl-1H-[1,2,3]triazol-4-yl]-(6-chloro-imidazo[1,5-a]pyridin-5-yl)-methanol;
(R)-(6-Cyclopropyl-imidazo[1,5-a]pyrazin-5-yl)-[1-(4-pyrrolidin-1-yl-phenyl)-1H-[1,2,3]triazol-4-yl]-methanol;
(R)-[1-(4-Amino-phenyl)-1H-[1,2,3]triazol-4-yl]-(6-cyclopropyl-imidazo[1,5-a]pyrazin-5-yl)-methanol;
(R)-(6-Cyclopropyl-imidazo[1,5-a]pyrazin-5-yl)-[1-(4-methylamino-phenyl)-1H-[1,2,3]triazol-4-yl]-methanol;
(R)-(6-Chloro-imidazo[1,5-a]pyridin-5-yl)-[1-(4-methoxy-phenyl)-5-methyl-1H-[1,2,3]triazol-4-yl]-methanol;
2-Chloro-4-{4-[(6-chloro-imidazo[1,5-a]pyridin-5-yl)-hydroxy-methyl]-5-methyl-[1,2,3]triazol-1-yl}-phenol;
(R)-(6-Cyclopropyl-imidazo[1,5-a]pyrazin-5-yl)-[1-(4-dimethylamino-phenyl)-1H-[1,2,3]triazol-4-yl]-methanol;
(R)-(6-Cyclopropyl-imidazo[1,5-a]pyrazin-5-yl)-{1-[4-(2-oxa-6-aza-spiro[3.3]hept-6-yl)-phenyl]-1H-[1,2,3]triazol-4-yl}-methanol;
(R)-(6-Cyclopropyl-imidazo[1,5-a]pyrazin-5-yl)-{1-[4-(6-oxa-1-aza-spiro[3.3]hept-1-yl)-phenyl]-1H-[1,2,3]triazol-4-yl}-methanol;
(R)-(6-Cyclopropyl-imidazo[1,5-a]pyrazin-5-yl)-[1-(1-methyl-2,3-dihydro-1H-indol-5-yl)-1H-[1,2,3]triazol-4-yl]-methanol;
4-{4-[(6-Chloro-imidazo[1,5-a]pyridin-5-yl)-hydroxy-methyl]-5-methyl-[1,2,3]triazol-1-yl}-2-fluoro-phenol;
4-(2-Chloro-4-{4-[(6-chloro-imidazo[1,5-a]pyridin-5-yl)-hydroxy-methyl]-5-methyl-[1,2,3]triazol-1-yl}-phenoxy)-2-methyl-butan-2-ol;
4-(4-{4-[(6-Chloro-imidazo[1,5-a]pyridin-5-yl)-hydroxy-methyl]-5-methyl-[1,2,3]triazol-1-yl}-2-fluoro-phenoxy)-2-methyl-butan-2-ol;
(6-Chloro-imidazo[1,5-a]pyridin-5-yl)-[5-chloro-1-(4-methoxy-phenyl)-1H-[1,2,3]triazol-4-yl]-methanol;
(R)-(6-Cyclopropyl-imidazo[1,5-a]pyrazin-5-yl)-{1-[2,5-difluoro-4-(3-fluoro-oxetan-3-ylmethoxy)-phenyl]-1H-[1,2,3]triazol-4-yl}-methanol;
(R)-(6-Cyclopropyl-imidazo[1,5-a]pyrazin-5-yl)-{1-[2,5-difluoro-4-(oxetan-3-ylmethoxy)-phenyl]-1H-[1,2,3]triazol-4-yl}-methanol;
(6-Cyclopropyl-imidazo[1,5-a]pyrazin-5-yl)-[1-(2,4-difluoro-phenyl)-5-methyl-1H-[1,2,3]triazol-4-yl]-methanol;
(6-Cyclopropyl-imidazo[1,5-a]pyrazin-5-yl)-[1-(2,5-difluoro-4-methoxy-phenyl)-5-methyl-1H-[1,2,3]triazol-4-yl]-methanol;
1-(4-{4-[(R)-(6-Cyclopropyl-imidazo[1,5-a]pyrazin-5-yl)-hydroxy-methyl]-[1,2,3]triazol-1-yl}-2,5-difluoro-phenoxy)-2-methyl-propan-2-ol; and
(6-Cyclopropyl-imidazo[1,5-a]pyrazin-5-yl)-[1-(2-fluoro-phenyl)-5-methyl-1H-[1,2,3]triazol-4-yl]-methanol.

23) Another embodiment relates to a compound according to any one of embodiments 1) or 2) selected from a group consisting of:
(R)-(6-Chloro-imidazo[1,5-a]pyridin-5-yl)-(1-phenyl-1H-[1,2,3]triazol-4-yl)-methanol;
(R)-(6-Methyl-imidazo[1,5-a]pyridin-5-yl)-(1-phenyl-1H-[1,2,3]triazol-4-yl)-methanol;
(R)-(6-Cyclopropyl-imidazo[1,5-a]pyrazin-5-yl)-(1-phenyl-1H-[1,2,3]triazol-4-yl)-methanol;
2-Chloro-4-{4-[(R)-(6-cyclopropyl-imidazo[1,5-a]pyridin-5-yl)-hydroxy-methyl]-[1,2,3]triazol-1-yl}-phenol;
(R)-[1-(3-Chloro-4-methoxy-phenyl)-1H-[1,2,3]triazol-4-yl]-(6-cyclopropyl-imidazo[1,5-a]pyridin-5-yl)-methanol;
(R)-[1-(3-Chloro-4-methoxy-phenyl)-1H-[1,2,3]triazol-4-yl]-(6-cyclopropyl-imidazo[1,5-a]pyrazin-5-yl)-methanol;
(R)-(6-Cyclopropyl-imidazo[1,5-a]pyrazin-5-yl)-[1-(3-fluoro-4-methoxy-phenyl)-1H-[1,2,3]triazol-4-yl]-methanol;
(R)-(6-Cyclopropyl-imidazo[1,5-a]pyrazin-5-yl)-[1-(2,5-difluoro-4-methoxy-phenyl)-1H-[1,2,3]triazol-4-yl]-methanol;
2-Chloro-4-{4-[(R)-(6-cyclopropyl-imidazo[1,5-a]pyrazin-5-yl)-hydroxy-methyl]-[1,2,3]triazol-1-yl}-phenol;
(R)-[1-(3-Bromo-4-methoxy-phenyl)-1H-[1,2,3]triazol-4-yl]-(6-cyclopropyl-imidazo[1,5-a]pyrazin-5-yl)-methanol;
(R)-(6-Cyclopropyl-imidazo[1,5-a]pyrazin-5-yl)-[1-(4-methoxymethyl-phenyl)-1H-[1,2,3]triazol-4-yl]-methanol;
(R)-[1-(5-Chloro-2-fluoro-4-methoxy-phenyl)-1H-[1,2,3]triazol-4-yl]-(6-cyclopropyl-imidazo[1,5-a]pyrazin-5-yl)-methanol;
(R)-[1-(3-Chloro-5-fluoro-4-methoxy-phenyl)-1H-[1,2,3]triazol-4-yl]-(6-cyclopropyl-imidazo[1,5-a]pyrazin-5-yl)-methanol;
4-{4-[(R)-(6-Cyclopropyl-imidazo[1,5-a]pyrazin-5-yl)-hydroxy-methyl]-[1,2,3]triazol-1-yl}-2-fluoro-phenol;
(R)-(6-Cyclopropyl-imidazo[1,5-a]pyrazin-5-yl)-{1-[3-fluoro-4-(3-fluoro-oxetan-3-ylmethoxy)-phenyl]-1H-[1,2,3]triazol-4-yl}-methanol;
1-(4-{4-[(R)-(6-Cyclopropyl-imidazo[1,5-a]pyrazin-5-yl)-hydroxy-methyl]-[1,2,3]triazol-1-yl}-2-fluoro-phenoxy)-2-methyl-propan-2-ol;
(R)-(6-Chloro-imidazo[1,5-a]pyridin-5-yl)-[1-(4-methoxy-phenyl)-1H-[1,2,3]triazol-4-yl]-methanol;
(R)-(6-Chloro-imidazo[1,5-a]pyridin-5-yl)-[1-(2,5-difluoro-4-methoxy-phenyl)-1H-[1,2,3]triazol-4-yl]-methanol;
(R)-[1-(3-Chloro-2-fluoro-4-methoxy-phenyl)-1H-[1,2,3]triazol-4-yl]-(6-cyclopropyl-imidazo[1,5-a]pyrazin-5-yl)-methanol;
(R)-(6-Ethyl-imidazo[1,5-a]pyridin-5-yl)-[1-(4-methoxy-phenyl)-1H-[1,2,3]triazol-4-yl]-methanol;
(R)-[1-(2,5-Difluoro-4-methoxy-phenyl)-1H-[1,2,3]triazol-4-yl]-(6-ethyl-imidazo[1,5-a]pyridin-5-yl)-methanol; and
(R)-(6-Cyclopropyl-imidazo[1,5-a]pyrazin-5-yl)-{1-[3-fluoro-4-(oxetan-3-ylmethoxy)-phenyl]-1H-[1,2,3]triazol-4-yl}-methanol.

24) Another embodiment relates to a compound according to embodiment 1) selected from a group consisting of:
(R)-(6-Cyclopropyl-imidazo[1,5-a]pyrazin-5-yl)-[1-(4-pyrrolidin-1-yl-phenyl)-1H-[1,2,3]triazol-4-yl]-methanol;
(R)-[1-(4-Amino-phenyl)-1H-[1,2,3]triazol-4-yl]-(6-cyclopropyl-imidazo[1,5-a]pyrazin-5-yl)-methanol;
(R)-(6-Cyclopropyl-imidazo[1,5-a]pyrazin-5-yl)-[1-(4-methylamino-phenyl)-1H-[1,2,3]triazol-4-yl]-methanol;
(R)-(6-Chloro-imidazo[1,5-a]pyridin-5-yl)-[1-(4-methoxy-phenyl)-5-methyl-1H-[1,2,3]triazol-4-yl]-methanol;
(R)-(6-Cyclopropyl-imidazo[1,5-a]pyrazin-5-yl)-[1-(4-dimethylamino-phenyl)-1H-[1,2,3]triazol-4-yl]-methanol;
(R)-(6-Cyclopropyl-imidazo[1,5-a]pyrazin-5-yl)-{1-[4-(2-oxa-6-aza-spiro[3.3]hept-6-yl)-phenyl]-1H-[1,2,3]triazol-4-yl}-methanol;
(R)-(6-Cyclopropyl-imidazo[1,5-a]pyrazin-5-yl)-{1-[4-(6-oxa-1-aza-spiro[3.3]hept-1-yl)-phenyl]-1H-[1,2,3]triazol-4-yl}-methanol;
(R)-(6-Cyclopropyl-imidazo[1,5-a]pyrazin-5-yl)-[1-(1-methyl-2,3-dihydro-1H-indol-5-yl)-1H-[1,2,3]triazol-4-yl]-methanol;
(R)-(6-Cyclopropyl-imidazo[1,5-a]pyrazin-5-yl)-{1-[2,5-difluoro-4-(3-fluoro-oxetan-3-ylmethoxy)-phenyl]-1H-[1,2,3]triazol-4-yl}-methanol;
(R)-(6-Cyclopropyl-imidazo[1,5-a]pyrazin-5-yl)-{1-[2,5-difluoro-4-(oxetan-3-ylmethoxy)-phenyl]-1H-[1,2,3]triazol-4-yl}-methanol; and
1-(4-{4-[(R)-(6-Cyclopropyl-imidazo[1,5-a]pyrazin-5-yl)-hydroxy-methyl]-[1,2,3]triazol-1-yl}-2,5-difluoro-phenoxy)-2-methyl-propan-2-ol.

Based on the dependencies of the different embodiments 1) to 20) as disclosed hereinabove, the following embodiments are thus possible and intended, and herewith specifically disclosed in individualized form:

2+1, 3+1, 3+2+1, 5+1, 5+2+1, 5+3+1, 5+3+2+1, 7+1, 7+2+1, 7+3+1, 7+3+2+1, 7+5+1, 7+5+2+1, 7+5+3+1, 7+5+3+2+1, 10+1, 10+2+1, 10+3+1, 10+3+2+1, 10+5+1, 10+5+2+1, 10+5+3+1, 10+5+3+2+1, 10+7+1, 10+7+2+1, 10+7+3+1, 10+7+3+2+1, 10+7+5+1, 10+7+5+2+1, 10+7+5+3+1, 10+7+5+3+2+1, 12+1, 12+2+1, 12+3+1, 12+3+2+1, 12+5+1, 12+5+2+1, 12+5+3+1, 12+5+3+2+1, 12+7+1, 12+7+2+1, 12+7+3+1, 12+7+3+2+1, 12+7+5+1, 12+7+5+2+1, 12+7+5+3+1, 12+7+5+3+2+1, 12+10+1, 12+10+2+1, 12+10+3+1, 12+10+3+2+1, 12+10+5+1, 12+10+5+2+1, 12+10+5+3+1, 12+10+5+3+2+1, 12+10+7+1, 12+10+7+2+1, 12+10+7+3+1, 12+10+7+3+2+1, 12+10+7+5+1, 12+10+7+5+2+1, 12+10+7+5+3+1, 12+10+7+5+3+2+1, 13+1, 13+2+1, 13+3+1, 13+3+2+1, 13+5+1, 13+5+2+1, 13+5+3+1, 13+5+3+2+1, 13+7+1, 13+7+2+1, 13+7+3+1, 13+7+3+2+1, 13+7+5+1, 13+7+5+2+1, 13+7+5+3+1, 13+7+5+3+2+1, 13+10+1, 13+10+2+1, 13+10+3+1, 13+10+3+2+1, 13+10+5+1, 13+10+5+2+1, 13+10+5+3+1, 13+10+5+3+2+1, 13+10+7+1, 13+10+7+2+1, 13+10+7+3+1, 13+10+7+3+2+1, 13+10+7+5+1, 13+10+7+5+2+1, 13+10+7+5+3+1, 13+10+7+5+3+2+1, 13+12+1, 13+12+2+1, 13+12+3+1, 13+12+3+2+1, 13+12+5+1, 13+12+5+2+1, 13+12+5+3+1, 13+12+5+3+2+1, 13+12+7+1, 13+12+7+2+1, 13+12+7+3+1, 13+12+7+3+2+1, 13+12+7+5+1, 13+12+7+5+2+1, 13+12+7+5+3+1, 13+12+7+5+3+2+1, 13+12+10+1, 13+12+10+2+1, 13+12+10+3+1, 13+12+10+3+2+1, 13+12+10+5+1, 13+12+10+5+2+1, 13+12+10+5+3+1, 13+12+10+5+3+2+1, 13+12+10+7+1, 13+12+10+7+2+1, 13+12+10+7+3+1, 13+12+10+7+3+2+1, 13+12+10+7+5+1, 13+12+10+7+5+2+1, 13+12+10+7+5+3+1, 13+12+10+7+5+3+2+1, 15+1, 15+2+1, 15+3+1, 15+3+2+1, 15+5+1, 15+5+2+1, 15+5+3+1, 15+5+3+2+1, 15+7+1, 15+7+2+1, 15+7+3+1, 15+7+3+2+1, 15+7+5+1, 15+7+5+2+1, 15+7+5+3+1, 15+7+5+3+2+1, 15+10+1, 15+10+2+1, 15+10+3+1, 15+10+3+2+1, 15+10+5+1, 15+10+5+2+1, 15+10+5+3+1, 15+10+5+3+2+1, 15+10+7+1, 15+10+7+2+1, 15+10+7+3+1, 15+10+7+3+2+1, 15+10+7+5+1, 15+10+7+5+2+1, 15+10+7+5+3+1, 15+10+7+5+3+2+1, 20+1, 20+2+1, 20+3+1, 20+3+2+1, 20+5+1, 20+5+2+1, 20+5+3+1, 20+5+3+2+1, 20+7+1, 20+7+2+1, 20+7+3+1, 20+7+3+2+1, 20+7+5+1, 20+7+5+2+1, 20+7+5+3+1, 20+7+5+3+2+1, 20+10+1, 20+10+2+1, 20+10+3+1, 20+10+3+2+1, 20+10+5+1, 20+10+5+2+1, 20+10+5+3+1, 20+10+5+3+2+1, 20+10+7+1, 20+10+7+2+1, 20+10+7+3+1, 20+10+7+3+2+1, 20+10+7+5+1, 20+10+7+5+2+1, 20+10+7+5+3+1, 20+10+7+5+3+2+1, 20+13+1, 20+13+2+1, 20+13+3+1, 20+13+3+2+1, 20+13+5+1, 20+13+5+2+1, 20+13+5+3+1, 20+13+5+3+2+1, 20+13+7+1, 20+13+7+2+1, 20+13+7+3+1, 20+13+7+3+2+1, 20+13+7+5+1, 20+13+7+5+2+1, 20+13+7+5+3+1, 20+13+7+5+3+2+1, 20+13+10+1, 20+13+10+2+1, 20+13+10+3+1, 20+13+10+3+2+1, 20+13+10+5+1, 20+13+10+5+2+1, 20+13+10+5+3+1, 20+13+10+5+3+2+1, 20+13+10+7+1, 20+13+10+7+2+1, 20+13+10+7+3+1, 20+13+10+7+3+2+1, 20+13+10+7+5+1, 20+13+10+7+5+2+1, 20+13+10+7+5+3+1, 20+13+10+7+5+3+2+1, 20+13+12+1, 20+13+12+2+1, 20+13+12+3+1, 20+13+12+3+2+1, 20+13+12+5+1, 20+13+12+5+2+1, 20+13+12+5+3+1, 20+13+12+5+3+2+1, 20+13+12+7+1, 20+13+12+7+2+1, 20+13+12+7+3+1, 20+13+12+7+3+2+1, 20+13+12+7+5+1, 20+13+12+7+5+2+1, 20+13+12+7+5+3+1, 20+13+12+7+5+3+2+1, 20+13+12+10+1, 20+13+12+10+2+1, 20+13+12+10+3+1, 20+13+12+10+3+2+1, 20+13+12+10+5+1, 20+13+12+10+5+2+1, 20+13+12+10+5+3+1, 20+13+12+10+5+3+2+1, 20+13+12+10+7+1, 20+13+12+10+7+2+1, 20+13+12+10+7+3+1, 20+13+12+10+7+3+2+1, 20+13+12+10+7+5+1, 20+13+12+10+7+5+2+1, 20+13+12+10+7+5+3+1, 20+13+12+10+7+5+3+2+1, 20+15+1, 20+15+2+1, 20+15+3+1, 20+15+3+2+1, 20+15+5+1, 20+15+5+2+1, 20+15+5+3+1, 20+15+5+3+2+1, 20+15+7+1, 20+15+7+2+1, 20+15+7+3+1, 20+15+7+3+2+1, 20+15+7+5+1, 20+15+7+5+2+1, 20+15+7+5+3+1, 20+15+7+5+3+2+1, 20+15+10+1, 20+15+10+2+1, 20+15+10+3+1, 20+15+10+3+2+1, 20+15+10+5+1, 20+15+10+5+2+1, 20+15+10+5+3+1, 20+15+10+5+3+2+1, 20+15+10+7+1, 20+15+10+7+2+1, 20+15+10+7+3+1, 20+15+10+7+3+2+1, 20+15+10+7+5+1, 20+15+10+7+5+2+1, 20+15+10+7+5+3+1, 20+15+10+7+5+3+2+1.

In the list above the numbers refer to the embodiments according to their numbering provided hereinabove whereas “+” indicates the dependency from another embodiment. The different individualized embodiments are separated by commas. In other words, “3+2+1” for example refers to embodiment 3) depending on embodiment 2), depending on embodiment 1), i.e. embodiment “3+2+1” corresponds to embodiment 1) further characterized by the features of the embodiments 2) and 3).

The compounds of Formula (I) encompass compounds with at least one (i.e. the asymmetric carbon atom to which the fragment [1,2,3]triazol-1,4-diyl is attached) and possibly more asymmetric centers, such as one or more asymmetric carbon atoms, which are allowed to be present in (R)- as well as (S)-configuration. The compounds of Formula (I) may further encompass compounds with one or more double bonds which are allowed to be present in Z- as well as E-configuration and/or compounds with substituents at a ring system which are allowed to be present, relative to each other, in cis- as well as trans-configuration. The compounds of Formula (I) may thus be present as mixtures of stereoisomers or preferably in stereoisomerically enriched form, especially as essentially pure stereoisomers. In Formula (II), in addition to the asymmetric carbon atom to which the fragment [1,2,3]triazol-1,4-diyl is attached and which has the defined absolute configuration shown in Formula (II), the compounds of said formula may contain further asymmetric carbon atoms which are allowed to be present in (R)- as well as (S)-configuration. The compounds of Formula (II) may thus be present as mixtures of stereoisomers or preferably as pure stereoisomers. Mixtures of stereoisomers may be separated in a manner known to a person skilled in the art.

In this patent application, a dotted line (e.g.

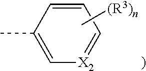

shows the point of attachment of the radical drawn.

In case a particular compound (or generic structure) is designated as (R)- or (S)-enantiomer, such designation is to be understood as referring to the respective compound (or generic structure) in enriched, especially essentially pure, enantiomeric form. Likewise, in case a specific asymmetric center in a compound is designated as being in (R)- or (S)-configuration or as being in a certain relative configuration, such designation is to be understood as referring to the compound that is in enriched, especially essentially pure, form with regard to the respective configuration of said asymmetric center. In analogy, cis- or trans-designations are to be understood as referring to the respective stereoisomer in enriched, especially essentially pure, form. Likewise, in case a particular compound (or generic structure) is designated as Z- or E-stereoisomer (or in case a specific double bond in a compound is designated as being in Z- or E-configuration), such designation is to be understood as referring to the respective compound (or generic structure) in enriched, especially essentially pure, stereoisomeric form (or to the compound that is in enriched, especially essentially pure, form with regard to the respective configuration of the double bond).

The term “enriched”, when used in the context of stereoisomers, is to be understood in the context of the present invention to mean that the respective stereoisomer is present in a ratio of at least 70:30, especially of at least 90:10 (i.e., in a purity of at least 70% by weight, especially of at least 90% by weight), with regard to the respective other stereoisomer/the entirety of the respective other stereoisomers.

The term “essentially pure”, when used in the context of stereoisomers, is to be understood in the context of the present invention to mean that the respective stereoisomer is present in a purity of at least 95% by weight, especially of at least 99% by weight, with regard to the respective other stereoisomer/the entirety of the respective other stereoisomers.

The present invention also includes isotopically labeled, especially ²H (deuterium) labeled compounds of Formula (I), which compounds are identical to the compounds of Formula (I) except that one or more atoms have each been replaced by an atom having the same atomic number but an atomic mass different from the atomic mass usually found in nature. Isotopically labeled, especially ²H (deuterium) labeled compounds of Formula (I) and salts thereof are within the scope of the present invention. Substitution of hydrogen with the heavier isotope ²H (deuterium) may lead to greater metabolic stability, resulting e.g. in increased in-vivo half-life or reduced dosage requirements, or may lead to a modified metabolism, resulting e.g. in an improved safety profile. In one embodiment of the invention, the compounds of Formula (I) are not isotopically labeled, or they are labeled only with one or more deuterium atoms. In a sub-embodiment, the compounds of Formula (I) are not isotopically labeled at all. Isotopically labeled compounds of Formula (I) may be prepared in analogy to the methods described hereinafter, but using the appropriate isotopic variation of suitable reagents or starting materials.

Where the plural form is used for compounds, salts, pharmaceutical compositions, diseases, this is intended to mean also a single compound, salt, composition and disease.

The term “modulate”, “modulation” or “modulator” used throughout the current text relate to an increase or to a decrease of the activity of an enzyme or a receptor. The term IDO and/or TDO inhibitor refers to an agent capable of inhibiting the activity of IDO and/or TDO enzymes.

Any reference hereinbefore or hereinafter to a compound of Formula (I) is to be understood as referring also to salts, especially pharmaceutically acceptable salts, of a compound of Formula (I), as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example ‘Handbook of Pharmaceutical Salts. Properties, Selection and Use.’, P. Heinrich Stahl, Camille G. Wermuth (Eds.), Wiley-VCH, 2008, and ‘Pharmaceutical Salts and Co-crystals’, Johan Wouters and Luc Quere (Eds.), RSC Publishing, 2012.

The compounds of Formula (I) and their pharmaceutically acceptable salts can be used as medicaments, e.g. in the form of pharmaceutical compositions for enteral (such as especially oral) or parenteral (including topical application or inhalation) administration.

The compounds of Formula (I) are suitable for inhibiting IDO and/or TDO enzymes, and for the prevention and/or treatment of diseases or disorders related to the IDO and/or TDO enzymes (such as especially cancers) in mammals, such as especially humans.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of Formula (I) or their pharmaceutically acceptable salts, optionally in combination with other therapeutically valuable substances, into a galenical administration form together with suitable, non-toxic, inert, pharmaceutically acceptable solid or liquid carrier materials and, if desired, usual pharmaceutical adjuvants.

In a preferred embodiment of the invention, the administered amount is comprised between 1 mg and 1000 mg per day, particularly between 5 mg and 500 mg per day, more particularly between 25 mg and 400 mg per day, especially between 50 mg and 200 mg per day.

Whenever the word “between” is used to describe a numerical range, it is to be understood that the end points of the indicated range are explicitly included in the range. For example: if a temperature range is described to be between 40° C. and 80° C., this means that the end points 40° C. and 80° C. are included in the range; or if a variable is defined as being an integer between 1 and 4, this means that the variable is the integer 1, 2, 3, or 4.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., and preferably to an interval extending from Y minus 5° C. to Y plus 5° C.

For avoidance of any doubt, if compounds are described as useful for the prevention or treatment of certain diseases, such compounds are likewise suitable for use in the preparation of a medicament for the prevention or treatment of said diseases.

The present invention also relates to a method for the prevention or treatment of a disease or disorder mentioned hereinabove comprising administering to a subject a pharmaceutically active amount of a compound of Formula (I) either alone or in combination with other pharmacologically active compounds and/or therapies.

The meaning of the term “prevention” may also be understood as “prophylaxis”.

One or more compounds of Formula (I) may be used in the prevention and/or treatment of diseases or disorders related to the IDO and/or TDO enzymes; such as especially cancers.

Cancers may be defined as including skin cancer including melanoma; metastatic melanoma; lung cancer including non-small cell lung cancer; bladder cancer including urinary bladder cancer; urothelial cell carcinoma; renal carcinomas including renal cell carcinoma; metastatic renal cell carcinoma; metastatic renal clear cell carcinoma; gastro-intestinal cancers including colorectal cancer; metastatic colorectal cancer; familial adenomatous polyposis (FAP); esophageal cancer; gastric cancer; gallbladder cancer; cholangiocarcinoma; hepatocellular carcinoma; and pancreatic cancer such as pancreatic adenocarcinoma or pancreatic ductal carcinoma; endometrial cancer; ovarian cancer; cervical cancer; neuroblastoma; prostate cancer including castrate-resistant prostate cancer; brain tumors including brain metastases, malignant gliomas, glioblastoma multiforme, medulloblastoma, meningiomas, neuroblastoma, astrocytoma; breast cancer including triple negative breast carcinoma; oral tumors; nasopharyngeal tumors; thoracic cancer; head and neck cancer; mesothelioma; leukemias including acute myeloid leukemia, adult T-cell leukemia; carcinomas; adenocarcinomas; thyroid carcinoma including papillary thyroid carcinoma; choriocarcinoma; sarcomas including Ewing's sarcoma; osteosarcoma; rhabdomyosarcoma; Kaposi's sarcoma; lymphoma including Burkitt's lymphoma, Hodgkin's lymphoma, MALT lymphoma; multiple myelomas; and virally induced tumors.

Further, cancers may be defined as including include brain cancers, skin cancers, bladder cancers, ovarian cancers, breast cancers, gastric cancers, pancreatic cancers, prostate cancers, colon cancers, blood cancers, lung cancers and bone cancers. Examples of such cancer types include neuroblastoma, intestine carcinoma such as rectum carcinoma, colon carcinoma, familiar adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, renal carcinoma, kidney parenchymal carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, diffuse large B-cell lymphoma (DLBCL), hepatocellular carcinoma, gall bladder carcinoma, bronchial carcinoma, small cell lung carcinoma, non-small cell lung carcinoma, multiple myeloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma and plasmacytoma.

Cancers may notably be defined as including skin cancer in particular advanced melanoma and Merkel cell carcinoma; lung cancer including non-small cell lung cancer; bladder cancer; head and neck cancer; renal cell cancer; Hodgkin's lymphoma; cervical cancer; endometrial cancer; breast cancer; colon cancer; gastrointestinal stromal tumors; pancreatic cancer; prostatic cancer; leukemia including acute myeloid leukemia; lymphoma; gastric cancer; ovarian cancer; esophageal carcinomas; hepatocarcinoma; and brain tumors in particular glioblastoma, mesothelioma, neuroblastoma, sarcoma in particular high-grade osteosarcoma, astrocytoma, myeloma.

Cancers may especially be defined as including solid tumors that have specific genetic features, called mismatch repair deficiency and high microsatellite instability; skin cancer, in particular advanced melanoma, Merkel cell carcinoma, and cutaneous squamous cell carcinoma; lung cancer (especially non-small cell lung cancer (NSCLC)); bladder cancer; advanced cervical cancer; advanced gastric cancer; head and neck cancer; renal cell carcinoma; metastatic colorectal cancer with mismatch repair deficiency (dMMR) or high microsatellite instability (MSI-H); primary mediastinal large B-cell lymphoma; advanced liver cancer; and Hodgkin's lymphoma.

One or more compounds of Formula (I) may be used in the prevention and/or treatment of any cancer, notably the cancers mentioned hereinabove, either alone, or in combination with further pharmacologically active compounds and/or therapies.

In addition to cancers, especially cancers as listed above, further diseases or disorders related to the IDO and/or TDO enzymes may be defined as including neurodegenerative disorders such as Parkinson's disease, Alzheimer's disease, Huntington's disease and Amyotrophic lateral sclerosis; Central nervous system (CNS) disorders such as Psychiatric disorders (schizophrenia, depression); pain; stroke; epilepsy; chronic infectious diseases such as HIV (AIDS including its manifestations such as cachexia, dementia and diarrhea) and HCV; infection and inflammation caused by various bacteria (such as Chlamydia strains and enteropathogenic strains), parasites (such as Trypanosoma, Leishmania, plasmodium) or viruses (such as influenza, human papilloma virus, cytomegalovirus, herpes simplex virus, Epstein-Barr virus, poliovirus, varicella zoster virus and coxsackie virus) as well as other infections (e.g. skin infections, GI infection, urinary tract infections, genito-urinary infections, systemic infections), autoimmune diseases including asthma, rheumatoid arthritis, multiple sclerosis, allergic inflammation, inflammatory bowel disease, psoriasis and systemic lupus erythematosus, organ transplantation (e.g. organ transplant rejection), metabolic disorders such as obesity, type 2 diabetes and/or fatty acid liver disease; cataracts; endometriosis; contraception and abortion.

Further autoimmune diseases include collagen diseases such as rheumatoid arthritis, systemic lupus erythematosus, Sharp's syndrome, CREST syndrome (calcinosis, Raynaud's syndrome, esophageal dysmotility, telangiectasia), dermatomyositis, vasculitis (Morbus Wegener's) and Sjogren's syndrome, renal diseases such as Goodpasture's syndrome, rapidly-progressing glomerulonephritis and membranoproliferative glomerulonephritis type II, endocrine diseases such as type-I diabetes, autoimmune polyendocrinopathy-candidiasis-ectodermal dystrophy (APECED), autoimmune parathyroidism, pernicious anemia, gonad insufficiency, idiopathic Morbus Addison's, hyperthyreosis, Hashimoto's thyroiditis and primary myxedema, skin diseases such as pemphigus vulgaris, bullous pemphigoid, herpes gestationis, epidermolysis bullosa and erythema multiforme major, liver diseases such as primary biliary cirrhosis, autoimmune cholangitis, autoimmune hepatitis type-1, autoimmune hepatitis type-2, primary sclerosing cholangitis, neuronal diseases such as multiple sclerosis, myasthenia gravis, myasthenic Lambert-Eaton syndrome, acquired neuromyotomy, Guillain-Barre syndrome (Muller-Fischer syndrome), stiff-man syndrome, cerebellar degeneration, ataxia, opsoclonus, sensoric neuropathy and achalasia, blood diseases such as autoimmune hemolytic anemia, idiopathic thrombocytopenic purpura (Morbus Werlhof), infectious diseases with associated autoimmune reactions such as AIDS, malaria and Chagas disease.

The terms “radiotherapy” or “radiation therapy” or “radiation oncology”, refer to the medical use of ionizing radiation in the prevention (adjuvant therapy) and/or treatment of cancer; including external and internal radiotherapy.

The term “targeted therapy” refers to the prevention/prophylaxis (adjuvant therapy) and/or treatment of cancer with one or more anti-neoplastic agents such as small molecules or antibodies which act on specific types of cancer cells or stromal cells. Some targeted therapies block the action of certain enzymes, proteins, or other molecules involved in the growth and spread of cancer cells. Other types of targeted therapies help the immune system kill cancer cells (immunotherapies); or deliver toxic substances directly to cancer cells and kill them. An example of a targeted therapy which is in particular suitable to be combined with the compounds of the present invention is immunotherapy, especially immunotherapy targeting the programmed death 1 (PD-1) receptor or its ligand PD-L1 (Feig C et al, PNAS 2013).

Immunotherapy further refers to (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators). Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-HI (PD-LI), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-IBBL, CD137 (4-IBB), TRAIL/Apo2-L, TRAILR1/DR4, TRAILR2/DR5, TRAILR3, TRAILR4, OPG, RANK, RANKL, TWEAKR/FnI4, TWEAK, BAFFR, EDAR, XEDAR, TACT, APRIL, BCMA, LTpR, LIGHT, DcR3, HVEM, VEGI/TLIA, TRAMP/DR3, EDAR, EDAI, XEDAR, EDA2, TNFRI, Lymphotoxin a/TNFp, TNFR2, TNFa, LTPR, Lymphotoxin a 1p2, FAS, FASL, RELT, DR6, TROY, NGFR.

When used in combination with the compounds of Formula (I), the term “targeted therapy” especially refers to agents such as: a) Epidermal growth factor receptor (EGFR) inhibitors or blocking antibodies (for example Gefitinib, Erlotinib, Afatinib, Icotinib, Lapatinib, Panitumumab, Zalutumumab, Nimotuzumab, Matuzumab and Cetuximab) as well as trastuzumab (HERCEPTIN); b) RAS/RAF/MEK pathway inhibitors (for example Vemurafenib, Sorafenib, Dabrafenib, GDC-0879, PLX-4720, LGX818, RG7304, Trametinib (GSK1120212), Cobimetinib (GDC-0973/XL518), Binimetinib (MEK162, ARRY-162), Selumetinib (AZD6244)); c) Janus kinase (JAK) inhibitors (for example Ruxolitinib, Itacitinib, Momelotinib); d) Aromatase inhibitors (for example Exemestane, Letrozole, Anastrozole, Vorozole, Formestane, Fadrozole); e); signal transduction inhibitors (STI). A “signal transduction inhibitor” is an agent that selectively inhibits one or more vital steps in signaling pathways, in the normal function of cancer cells, thereby leading to apoptosis. Suitable STis include but are not limited to: (i) bcr/abl kinase inhibitors such as, for example, STI 571 (GLEEVEC®), Dasatinib; (ii) epidermal growth factor (EGF) receptor inhibitors such as, for example, kinase inhibitors (IRESSA®, SSI-774) and antibodies (Imclone: C225 [Goldstein et al., Clin. Cancer Res., 1:1311-1318 (1995)], and Abgenix: ABX-EGF); (iii) her-2/neu receptor inhibitors such as famesyl transferase inhibitors (FTI) such as, for example, L-744,832 (Kohl et al., Nat. Med., 1(8):792-797 (1995)); (iv) inhibitors of Akt family kinases or the Akt pathway, such as, for example, rapamycin (see, for example, Sekulic et al., Cancer Res., 60:3504-3513 (2000)); (v) cell cycle kinase inhibitors such as, for example, flavopiridol and UCN-O1 (see, for example, Sausville, Curr. Med. Chem. Anti-Cane. Agents, 3:47-56 (2003)); and (vi) phosphatidyl inositol kinase inhibitors such as, for example, LY294002 (see, for example, Vlahos et al., J Biol. Chem., 269:5241-5248 (1994)). f) Angiogenesis inhibitors, especially VEGF signalling inhibitors such as Bevacuzimab (Avastin), Ramucirumab, Sorafenib or Axitinib; g) Immune Checkpoint inhibitors (for example: anti-PD1 antibodies such as Pembrolizumab (Lambrolizumab, MK-3475), Nivolumab, Pidilizumab (CT-011), AMP-514/MED10680, PDR001, SHR-1210; REGN2810, BGBA317, PF-06801591, MGA-012, TSR042, JS-001, BCD100, IBI-308, BI-754091; fusion proteins targeting PD-1 such as AMP-224; small molecule anti-PD1 agents such as for example compounds disclosed in WO2015/033299, WO2015/044900 and WO2015/034820; anti-PD1L antibodies, such as BMS-936559, atezolizumab (MPDL3280A, RG7446), avelumab (MSB0010718C), durvalumab (MED14736); anti-PDL2 antibodies, such as AMP224; anti-CTLA-4 antibodies, such as ipilimumab, tremelimumab; anti-Lymphocyte-activation gene 3 (LAG-3) antibodies, such as Relatlimab (BMS-986016), IMP701, IMP731, MK-4280, ImmuFact IMP321; anti T cell immunoglobulin mucin-3 (TIM-3) antibodies, such as MBG453, TSR-022; anti T cell immunoreceptor with Ig and ITIM domains (TIGIT) antibodies, such as RG6058 (anti-TIGIT, MTIG7192A); anti-Killer-cell immunoglobulin-like receptors (KIR) for example Lirilumab (IPH2102/BMS-986015), antagonists of Galectins (such as Galectin-1, Galectin-9), BTLA; h) Vaccination approaches (for example dendritic cell vaccination, DNA, peptide or protein vaccination (for example with gp100 peptide or MAGE-A3 peptide) as well as recombinant viruses; i) Re-introduction of patient derived or allogenic (non-self) cancer cells genetically modified to secrete immunomodulatory factors such as granulocyte monocyte colony stimulating factor (GMCSF) gene-transfected tumor cell vaccine (GVAX) or Fms-related tyrosine kinase 3 (Flt-3) ligand gene-transfected tumor cell vaccine (FVAX), or Toll like receptor enhanced GM-CSF tumor based vaccine (TEGVAX); j) T-cell based adoptive immunotherapies, including chimeric antigen receptor (CAR) engineered T-cells (for example CTL019); k) Cytokine or immunocytokine based therapy (for example Interferon alpha, interferon beta, interferon gamma, interleukin 2, interleukin 6, interleukin 10, interleukin 15, TGF-3); l) Toll-like receptor (TLR) agonists (for example resiquimod, imiquimod, motolimod, glucopyranosyl lipid A, CpG oligodesoxynucleotides); m) Thalidomide analogues (for example Lenalidomide, Pomalidomide); n) Activators of T-cell co-stimulatory receptors (for example anti-CD137/4-1BB antibodies, such as BMS-663513 (urelumab), Utomilumab (PF-05082566); anti-OX40/CD134 (Tumor necrosis factor receptor superfamily, member 4) (such as RG7888 (MOXR0916), 9B12; MEDI6469, GSK3174998, MEDI6383, MEDI0562), anti OX40-Ligand/CD252; anti-glucocorticoid-induced TNFR family related gene (GITR) (such as TRX518, MEDI1873, MK-4166, BMS-986156, BMS-986153), anti-CD40 (TNF receptor superfamily member 5) antibodies (such as Dacetuzumab (SGN-40), HCD122, CP-870,893, RG7876, ADC-1013, APX005M, SEA-CD40); anti-CD40-Ligand antibodies (such as BG9588); anti-CD27 antibodies such as Varlilumab; anti-CD28 antibodies; anti-ICOS antibodies; o) Molecules binding a tumor specific antigen as well as a T-cell surface marker such as bispecific antibodies or antibody fragments, antibody mimetic proteins such as designed ankyrin repeat proteins (DARPINS), bispecific T-cell engager (BITE, for example AMG103, AMG330); p) Antibodies or small molecular weight inhibitors targeting colony-stimulating factor-1 receptor (CSF-1R) (for example Emactuzumab (RG7155), Cabiralizumab (FPA-008), PLX3397). q) Agents targeting immune cell check points on natural killer cells such as antibodies against Killer-cell immunoglobulin-like receptors (KIR) for example Lirilumab (IPH2102/BMS-986015); r) Agents targeting the Adenosine receptors or the ectonucleases CD39 and CD73 that convert adenosin triphosphate (ATP) to Adenosine, such as MEDI9447 (anti-CD73 antibody), PBF-509; CPI-444 (Adenosine A2a receptor antagonist); s) antagonists to chemokine receptors including CCR2 or CCR4; t) modulators of the complement system v) agents that deplete or inhibit T regulatory cells (e.g., using an anti-CD25 monoclonal antibody (e.g., daclizumab) or by ex vivo anti-CD25 bead depletion) or reverse/prevent T cell anergy or exhaustion.

When used in combination with the compounds of Formula (I), immune checkpoint inhibitors such as those listed under f), and especially those targeting the programed cell death receptor 1 (PD-1 receptor) or its ligand PD-L1, are preferred.

The term “chemotherapy” refers to the treatment of cancer with one or more cytotoxic anti-neoplastic agents (“cytotoxic chemotherapy agents”). Chemotherapy is often used in conjunction with other cancer treatments, such as radiation therapy or surgery. The term especially refers to conventional chemotherapeutic agents which act by killing cells that divide rapidly, one of the main properties of most cancer cells. Chemotherapy may use one drug at a time (single-agent chemotherapy) or several drugs at once (combination chemotherapy or polychemotherapy). Chemotherapy using drugs that convert to cytotoxic activity only upon light exposure is called photochemotherapy or photodynamic therapy.

The term “cytotoxic chemotherapy agent” or “chemotherapy agent” as used herein refers to an active anti-neoplastic agent inducing apoptosis or necrotic cell death. When used in combination with the compounds of Formula (I), the term especially refers to conventional cytotoxic chemotherapy agents such as: 1) alkylating agents (including, without limitation, nitrogen mustards, ethylenimine derivatives, alkyl sulfonates, nitrosoureas and triazenes) such as uracil mustard, mechlorethamine, chlorambucil, cyclophosphamide, ifosfamide, streptozocin, carmustine, lomustine, melphalan, busulfan, procarbazine, dacarbazine, temozolomide, pipobroman, triethylene-melamine, triethylenethiophosphoramine, thiotepa or altretamine; in particular temozolomide); 2) platinum drugs (for example cisplatin, carboplatin or oxaliplatin); 3) antimetabolite drugs (for example 5-fluorouracil, floxuridine, pentostatine, capecitabine, 6-mercaptopurine, methotrexate, gemcitabine, cytarabine, fludarabine or pemetrexed); 4) anti-tumor antibiotics (for example daunorubicin, doxorubicin, epirubicin, idarubicin, actinomycin-D, bleomycin, mitomycin-C or mitoxantrone); 5) mitotic inhibitors (for example paclitaxel, docetaxel, ixabepilone, vinblastine, vincristine, vinorelbine, vindesine or estramustine); or 6) topoisomerase inhibitors (for example etoposide, teniposide, topotecan, irinotecan, diflomotecan or elomotecan). Also suitable are cytotoxic agents such as biological response modifiers; growth inhibitors; antihormonal therapeutic agents; leucovorin; tegafur; and haematopoietic growth factors.

When used in combination with the compounds of Formula (I), preferred cytotoxic chemotherapy agents are the above-mentioned alkylating agents (notably fotemustine, cyclophosphamide, ifosfamide, carmustine, dacarbazine and prodrugs thereof such as especially temozolomide or pharmaceutically acceptable salts of these compounds; in particular temozolomide); mitotic inhibitors (notably paclitaxel, docetaxel, ixabepilone; or pharmaceutically acceptable salts of these compounds; in particular paclitaxel); platinum drugs (notably cisplatin, oxaliplatin and carboplatin); as well etoposide and gemcitabine. 1) Chemotherapy may be given with a curative intent or it may aim to prolong life or to palliate symptoms. 2) Combined modality chemotherapy is the use of drugs with other cancer treatments, such as radiation therapy or surgery. 3) Induction chemotherapy is the first line treatment of cancer with a chemotherapeutic drug. This type of chemotherapy is used for curative intent. 4) Consolidation chemotherapy is the given after remission in order to prolong the overall disease-free time and improve overall survival. The drug that is administered is the same as the drug that achieved remission. 5) Intensification chemotherapy is identical to consolidation chemotherapy but a different drug than the induction chemotherapy is used. 6) Combination chemotherapy involves treating a patient with a number of different drugs simultaneously. The drugs differ in their mechanism and side effects. The biggest advantage is minimising the chances of resistance developing to any one agent. Also, the drugs can often be used at lower doses, reducing toxicity. 7) Neoadjuvant chemotherapy is given prior to a local treatment such as surgery, and is designed to shrink the primary tumor. It is also given to cancers with a high risk of micrometastatic disease. 8) Adjuvant chemotherapy is given after a local treatment (radiotherapy or surgery). It can be used when there is little evidence of cancer present, but there is risk of recurrence. It is also useful in killing any cancerous cells that have spread to other parts of the body. These micrometastases can be treated with adjuvant chemotherapy and can reduce relapse rates caused by these disseminated cells. 9) Maintenance chemotherapy is a repeated low-dose treatment to prolong remission. 10) Salvage chemotherapy or palliative chemotherapy is given without curative intent, but simply to decrease tumor load and increase life expectancy. For these regimens, a better toxicity profile is generally expected.

Preparation of Compounds of Formula (I):

The compounds of Formula (I) can be manufactured by the methods given below, by the methods given in the Examples or by analogous methods. Optimum reaction conditions may vary with the particular reactants or solvents used, but such conditions can be determined by a person skilled in the art by routine optimization procedures.

In the schemes below, the generic groups R¹, R², R³, R⁴, X₁, X₂ and n are as defined for the compounds of Formula (I). For avoidance of doubt, X refers to halogen or, when comprised in a heterocycle (e.g. as X₁ or X₂), it refers to nitrogen or carbon. In some instances, said generic groups may be incompatible with the assembly illustrated in the schemes, or will require the use of protecting groups (PG). The use of protecting groups is well known in the art (see for example “Protective Groups in Organic Synthesis”, T. W. Greene, P. G. M. Wuts, Wiley-Interscience, 1999). For the purposes of this discussion, it will be assumed that such protecting groups as necessary are in place. In some cases, the final product may be further modified, for example, by manipulation of substituents to give a new final product. These manipulations may include, but are not limited to, reduction, oxidation, alkylation, acylation, and hydrolysis reactions which are commonly known to those skilled in the art. The compounds obtained may also be converted into salts, especially pharmaceutically acceptable salts in a manner known in the art.

Compounds of the Formula (I) of the present invention can be prepared according to the general synthetic schemes as outlined below.

### Synthesis of Compounds of Formula (I)

Generally, compounds of Formula (I) where R²═H are obtained by reaction of a propargylic alcohol intermediate 1 with an azide 2 using standard copper-catalyzed azide-alkyne cycloaddition (CuAAC) conditions such as copper sulfate, ascorbic acid sodium salt in a mixture of polar solvents such as DMF and water at room temperature (Scheme 1). Azides can be prepared using standard methods (for example, from halides, boronic acids or amines).

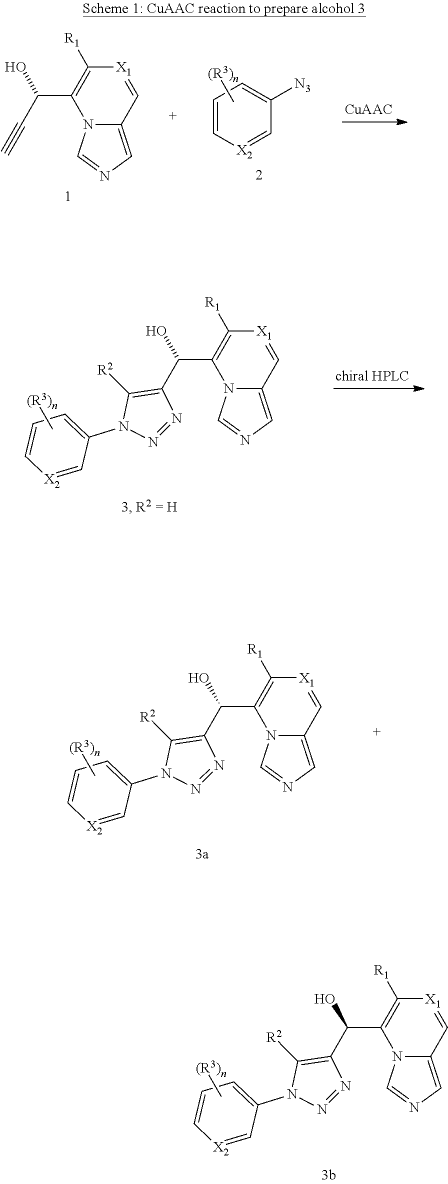

The enantiopure alcohols 3a and 3b can be obtained by chiral separation of the resulting product 3 of the CuAAC (Scheme 1).

Alternatively, enantiopure compounds of Formula (I) where R²═H can be obtained by CuAAC reaction of enantiopure propargylic alcohol 1a and a suitable azide 2 (Scheme 2). The enantiopure propargylic alcohol can be obtained by chiral separation of the racemic propargylic alcohol 1.

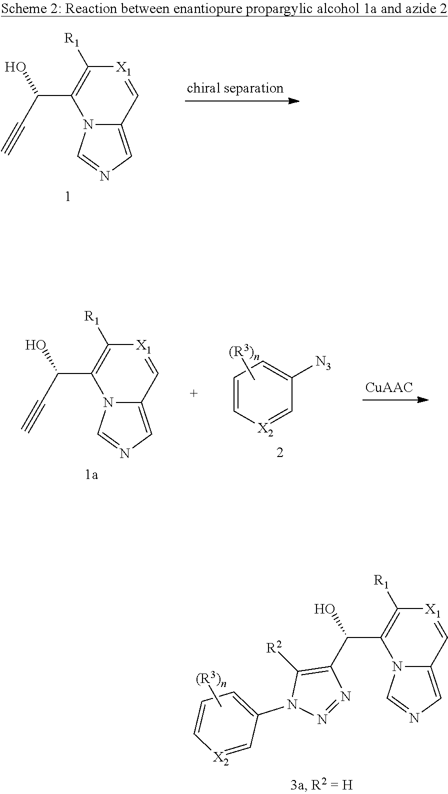

Alternatively, compound of Formula (I) where R²=Me can be obtained by reaction of a magnesium bromide species 5 with aldehyde 4 (Scheme 3). The Grignard reagent 5 can be prepared in situ using a suitable azide 2 and propynylmagnesium bromide.

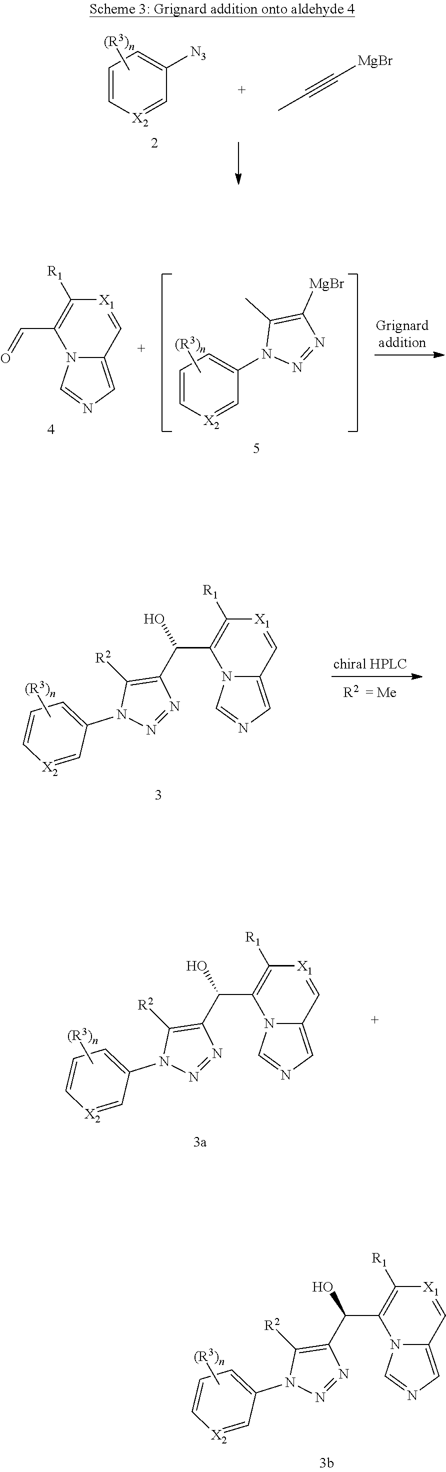

Alternatively, compounds of Formula (I) can be obtained by a deprotonation/addition sequence starting from intermediate 6 and a suitable aldehyde 7 (Scheme 4, for example with X₁=carbon). Compound 6 can be deprotonated using a base such as n-BuLi in a solvent such as THF and at a temperature ranging from −78° C. to 0° C. and the resulting anion can be treated with aldehyde 7 in a solvent such as THF and at a temperature ranging from −78° C. to RT. The racemic compounds can then be separated using chiral preparative HPLC to give alcohols 3a and 3b.

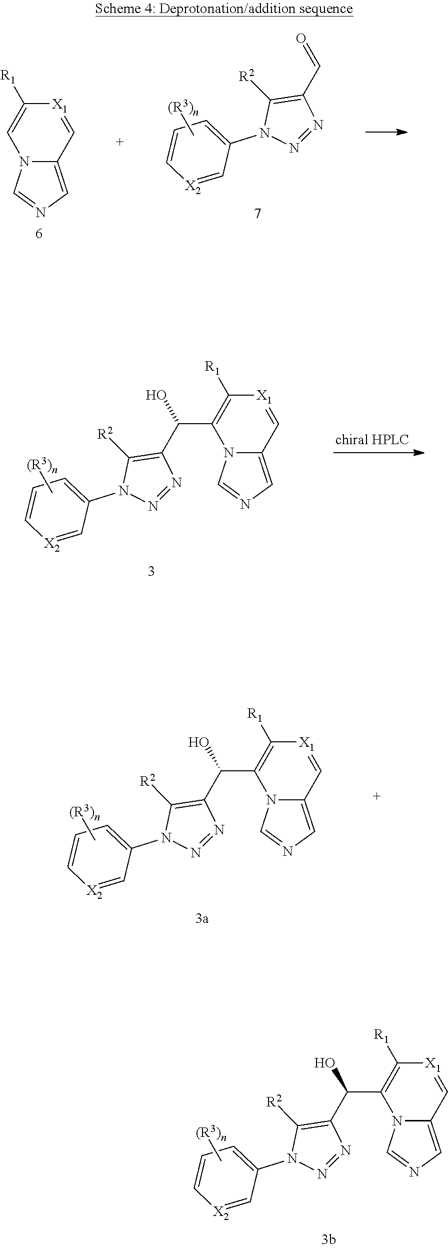

Alternatively, a protecting/directing group strategy can be used to prepare compounds of Formula (I) (Scheme 5). Deprotonation of intermediate 8 using a base such as n-BuLi or LDA in a solvent such as THF at a temperature around −78° C. or higher, and subsequent addition of a suitable aldehyde 7 give alcohol 9. Removal of the thioether function is performed using a catalyst such as Raney nickel in a solvent such as a mixture of ethanol and water, at a temperature ranging from RT to 90° C. to give compound 3. The racemic compounds can then be separated using chiral preparative HPLC to give alcohols 3a and 3b.

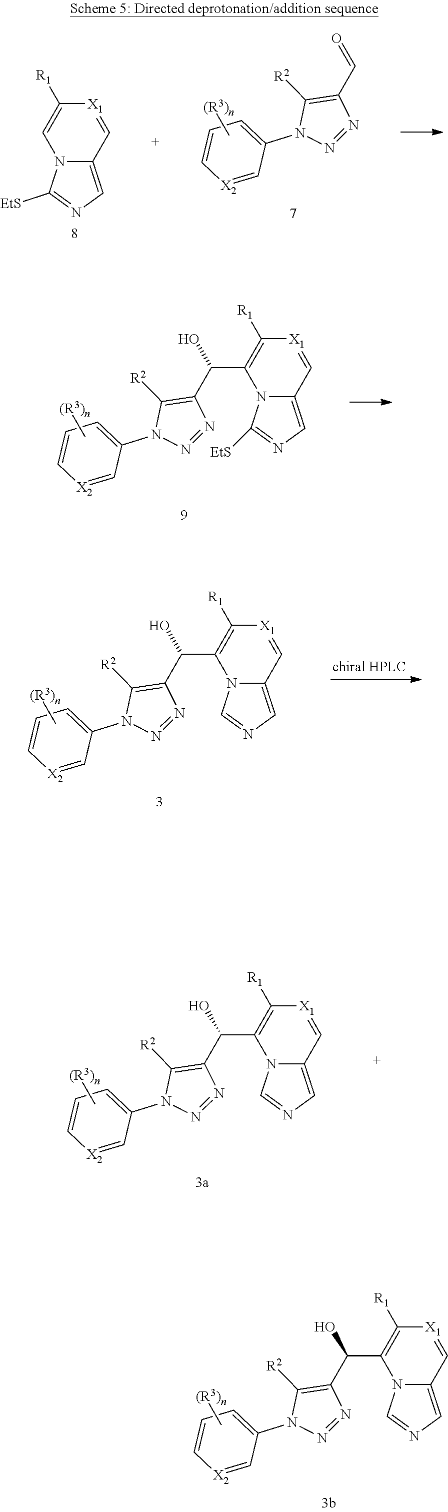

Alternatively, compounds of Formula (I) can be prepared by alkylation of 10 (Scheme 6) using standard alkylation conditions such as an halide (R⁴—X) in the presence of NaI and a base such as K₂CO₃ in a solvent such as DMF at a temperature ranging from RT to 100° C. The racemic compounds 11 can then be separated using chiral preparative HPLC to give alcohols 11a and 11b.

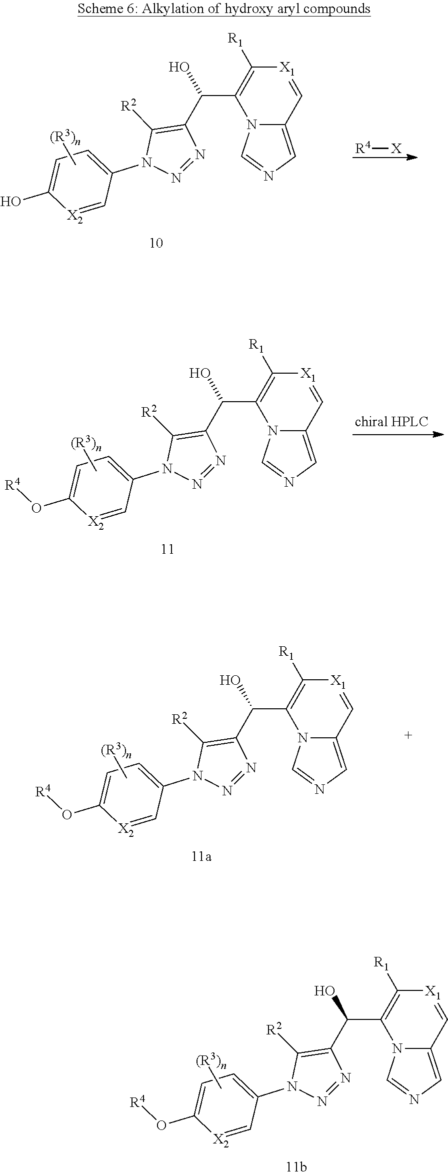

### Synthesis of Aldehyde 4 and Propargylic Alcohol Intermediate 1

Propargylic alcohol 1 can be prepared using the synthetic sequence described in Scheme 7 (for example with X₁=carbon).

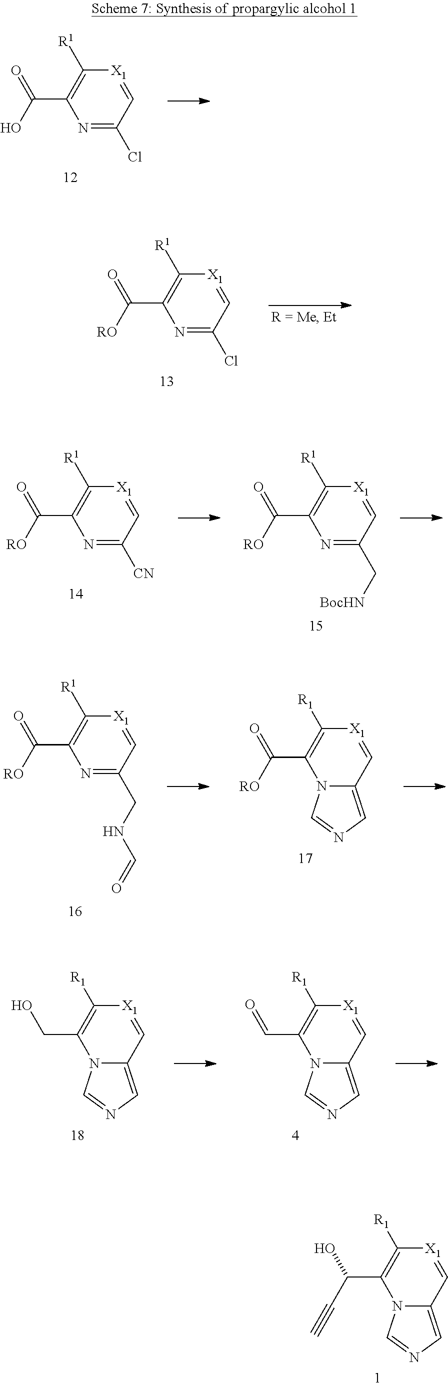

Starting from carboxylic acid 12, the acid function is converted into the corresponding methyl or ethyl ester using standard esterification conditions such as thionyl chloride in a solvent such as EtOH and a temperature around RT. The 2-Cl heteroaryl 13 is then converted into the corresponding 2-CN heteroaryl 14 by metal-catalyzed cyanation using Zn(CN)₂ as the cyanide source, a palladium catalyst such as Pd₂(dba)₃ and a ligand such as dppf, in a solvent such as DMF and at a temperature ranging from RT to 110° C. The nitrile function is reduced to the corresponding amine using Raney nickel under hydrogen atmosphere (generated for example in a HCube-Pro apparatus) in a solvent such as EtOH and in the presence of di-tert-butyl-dicarbonate in order to generate the Boc-protected amine 15. The protecting group is then removed and the primary amine converted into the corresponding formylated amine using a formylating agent such as ethylformate in the presence of a base such as DIPEA at a temperature ranging from RT to 50° C. Formylated amine 16 can then by cyclized into bicycling system 17 using a dehydrating agent such as POCl₃ in a solvent such as toluene or DCM and a temperature ranging from 0° C. to 110° C. The ester function is reduced into the corresponding alcohol using a reducing agent such as NaBH₄ in a solvent such as THF, MeOH or EtOH or a mixture. Alcohol 18 is oxidized to aldehyde 4 using an oxidizing agent such as Dess-Martin periodinane or MnO₂, in a solvent such as DCM, or CH₃CN and a temperature ranging from 0° C. to 70° C. Alternatively, aldehyde 4 can be obtained from ester 17 via Weinreb amide 22 (Scheme 8). Aldehyde 4 can be transformed into propargylic alcohol 1 via a Grignard reaction using ethynylmagnesium bromide in a solvent such as THF at a temperature ranging from 0° C. to RT.

An alternative synthetic pathway to propargylic alcohol 1 is shown in Scheme 8 (for example with X₁=nitrogen).

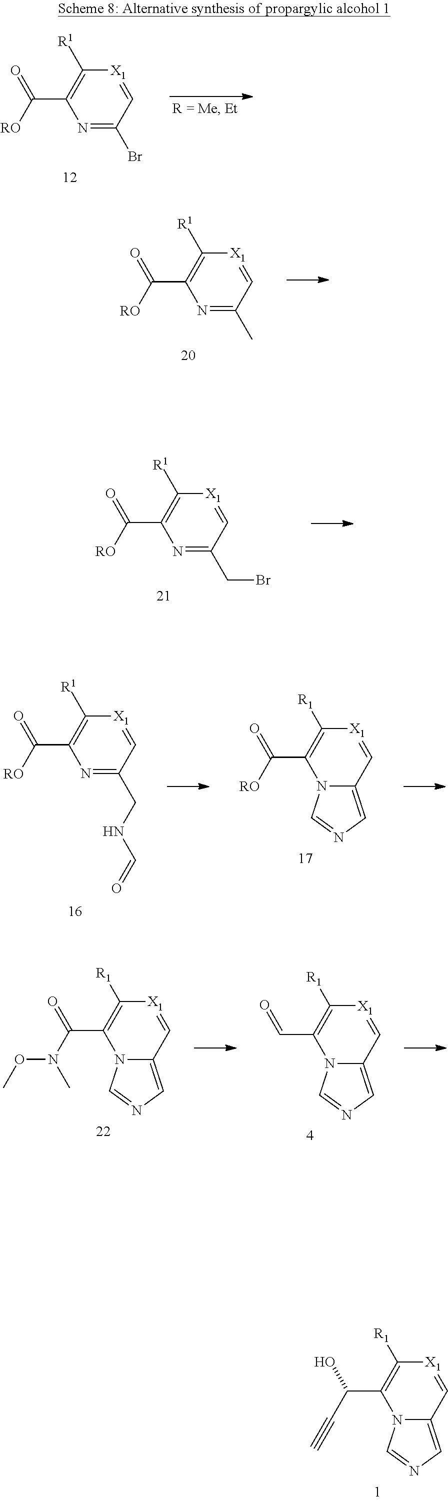

Commercially available (or prepared by esterification of the corresponding carboxylic acid, or by FGI to introduce R¹ substituent) bromide 19 is converted into bromo methyl 21 for example via bromination of methyl derivative 20 using standard radical bromination reaction conditions such as NBS in the presence of AIBN in a solvent such as CCl₄ and a temperature ranging from RT to 80° C. Methyl derivative, in turn, can be obtained by a cross-coupling reaction using for example trimethylboroxine, a palladium-based catalyst such as Pd(dppf)₂ in a solvent such as dioxane and in the presence of a base such as K₂CO₃ and at a temperature ranging from RT to 110° C. Bromide 21 can then be converted into formamide 16 using a two-step one-pot procedure involving the formation of a bis formamide by reaction with sodium diformamide in a solvent such as DMF and at a temperature around RT, followed by hydrolysis of one of the formyl groups under basic conditions, using for example NaHCO₃ as a base. Similarly to what is described in Scheme 7, formylated amine 16 can then by cyclized into bicyclic system 17 using a dehydrating agent such as POCl₃ in a solvent such as toluene or DCM and a temperature ranging from 0° C. to 110° C. The ester function is then converted into Weinreb amide 22 by saponification using a base such as LiOH in a mixture of solvents such as THF and water and at a temperature ranging from 0° C. to 50° C., followed by standard amide coupling using N,O-dimethylhydroxylamine, a coupling agent such as HATU in the presence of a base such as DIPEA in a solvent such as DMF at a temperature ranging from 0° C. to RT. Weinreb amide 22 can be reduced to aldehyde 4 using a reducing agent such as DIBALH in a solvent such as THF or toluene and a temperature ranging from −20° C. to RT. Aldehyde 4 can be transformed into propargylic alcohol 1 via a Grignard reaction using ethynylmagnesium bromide in a solvent such as THF at a temperature ranging from 0° C. to RT.

Alternatively, R¹ can be interconverted (Cl to cyclopropyl or methyl, or ethyl) at any appropriate stage of the syntheses displayed in Schemes 7 and 8. For example, aldehyde 4 can be converted into the corresponding intermediate where R¹ is an alkyl/cycloalkyl group (for example cyclopropyl, methyl or ethyl) using standard metal-catalyzed coupling reactions such as a Suzuki cross-coupling reaction (Scheme 9). Alternatively, the R¹ substituent can be introduced onto ester 17, using metal-catalyzed coupling reactions such as a Negishi cross-coupling reaction (Scheme 10).

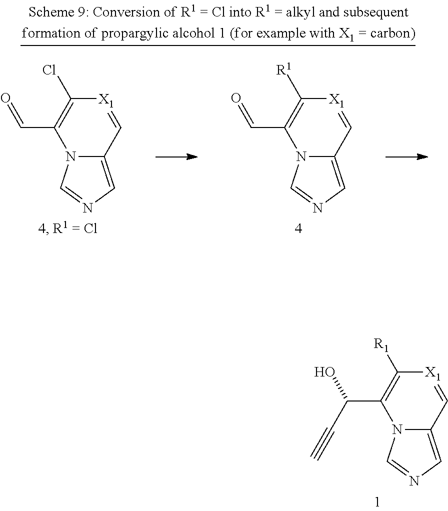

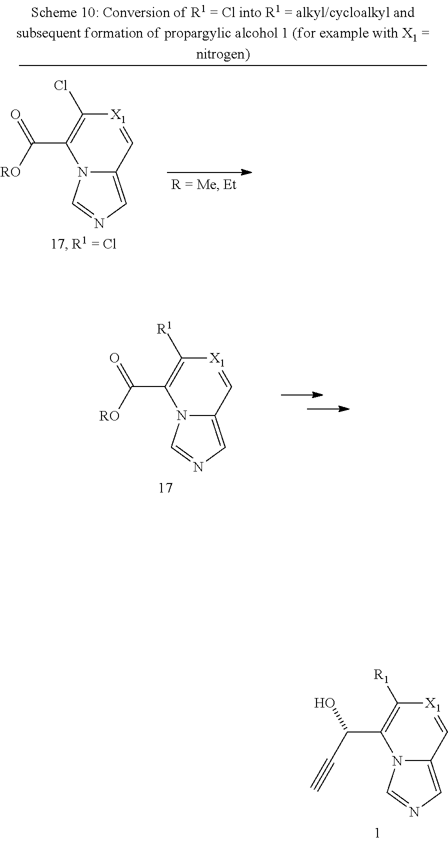

### Synthesis of Azides 2

Azides, if not commercially available, can be prepared using standard methods, for example starting from bromides, boronic acids (Chan-Lam coupling) or amines (Sandmeyer reaction), or by FGI of appropriately substituted azides.

Alternatively, azides 24 can be prepared by alkylation of 23 (Scheme 11) using standard alkylation conditions such as an halide (R⁴—X) in the presence of NaI and a base such as K₂CO₃ in a solvent such as DMF at a temperature ranging from RT to 50° C.

### Synthesis of Intermediate 6

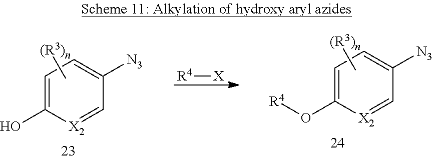

Primary amine 26 (either commercially available or prepared by functional group interconversion from the corresponding carboxylic acid or ester 25 (Scheme 12) or from the corresponding alcohol) is converted into the corresponding formylated amine 27, which can be cyclized into bicyclic system 6 using a dehydrating agent such as POCl₃ in a solvent such as toluene or DCM and a temperature ranging from 0° C. to 110° C.

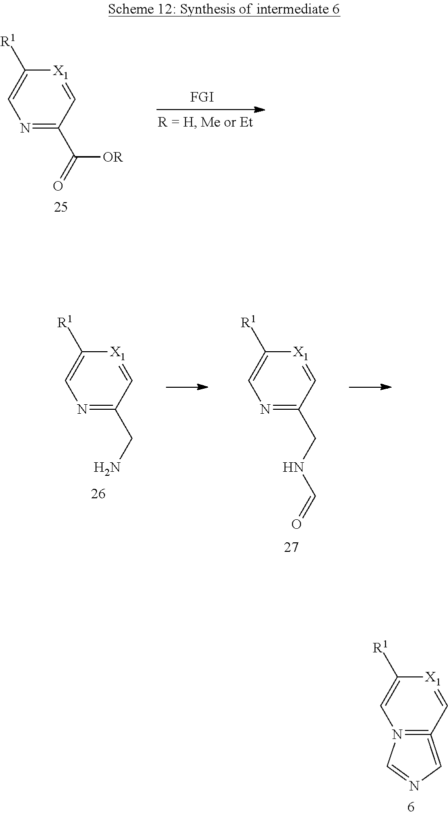

### Synthesis of Intermediate 8

Amine 26 can be cyclized into thiol 28 using standard conditions such as carbon disulfide in the presence of a base such as Et₃N, in a solvent such as MeOH and at a temperature ranging from 0° C. to 70° C. (Scheme 13). Thiol 28 can be alkylated using standard alkylation conditions such as EtI in the presence of a base such as K₂CO₃ in a solvent such as acetone and at a temperature ranging from RT to 45° C. to give intermediate 8.

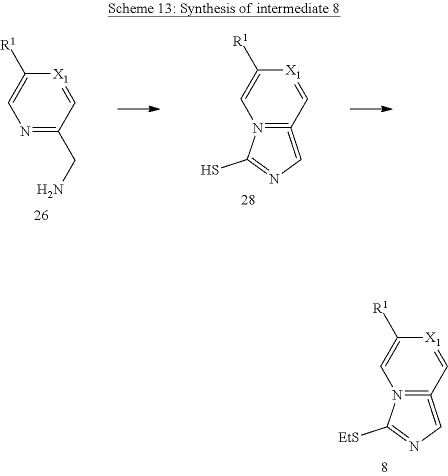

### Synthesis of Aldehyde 7

Aldehyde 7, if not commercially available, can be prepared by standard methods, two of them are described in Scheme 14 and Scheme 15. Aldehyde 7 can be prepared by base-catalyzed cycloaddition reaction between appropriately substituted beta-keto ester 29 and azide 2 (Scheme 14). Ester 30 can be reduced to the alcohol, which can be subsequently oxidized to said aldehyde. Alternatively, ester 30 can be transformed into the corresponding Weinreb amide, which can in turn be reduced to aldehyde 7.

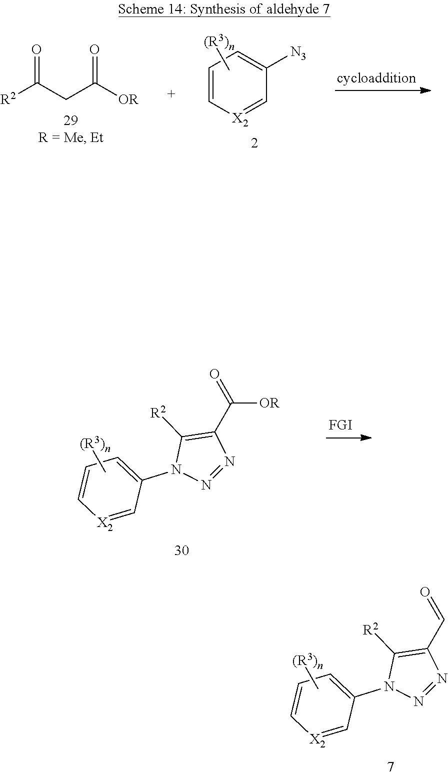

Alternatively, typical copper-catalyzed alkyne-azide coupling reaction can be used to couple alkyne 31 and azide 2 (Scheme 15). The resulting ester can be converted into the corresponding aldehyde using the methods described above.

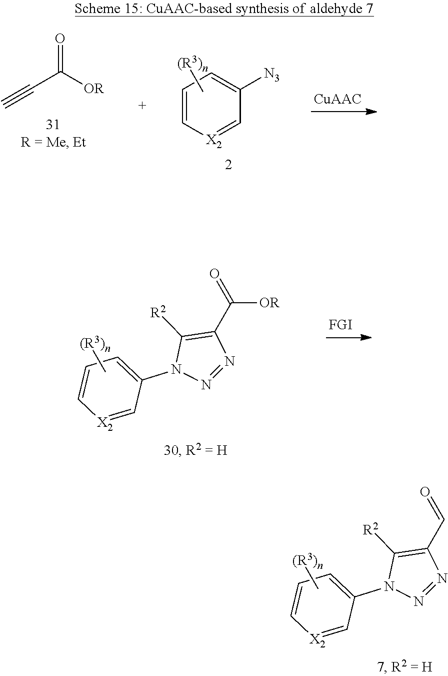

Whenever the compounds of Formula (I) are obtained in the form of mixtures of enantiomers, the enantiomers can be separated using methods known to one skilled in the art: e.g. by formation and separation of diastereomeric salts or by HPLC over a chiral stationary phase such as a Regis Whelk-O1(R,R) (10 μm) column, a Daicel ChiralCel OD-H (5-10 μm) column, or a Daicel ChiralPak IA (10 μm), IA, IB, IC, IF, or IF (5 μm) or AD-H (5 μm) column. Typical conditions of chiral HPLC are an isocratic mixture of eluent A (EtOH, in presence or absence of an amine such as triethylamine or diethylamine) and eluent B (heptane), at a flow rate of 0.8 to 150 mL/min.

The following examples are provided to illustrate the invention. These examples are illustrative only and should not be construed as limiting the invention in any way.

### EXPERIMENTAL PART

Chemistry

All temperatures are stated in ° C.

Preparative HPLC Conditions:

The conditions for preparative HPLC purifications were chosen among the possibilities given below depending on the properties of the compounds to be purified. More than one option per problem can lead to a successful result. Equipment: HPLC pumps: Gilson 333/334 or equivalent Autosampler: Gilson LH215 (with Gilson 845z injector) or equivalent Degasser: Dionex SRD-3200 or equivalent Make-up pump: Dionex ISO-3100A or equivalent DAD detector: Dionex DAD-3000 or equivalent MS detector: Single quadrupole mass analyzer Thermo Finnigan MSQ Plus or equivalent MRA splitter: MRA100-000 flow splitter or equivalent ELS detector: Polymer Laboratories PL-ELS1000 or equivalent. Method: Column: variable Waters Atlantis T3 30×75 mm 10 μm (acidic conditions only); Waters XBridge C18, 30×75 mm 10 μm (acidic/basic conditions); Waters XBridge C18, 50×150 mm 10 μm (acidic/basic conditions); Flow rate: variable 75 mL/min (for columns with dimension 30×75 mm), 150 mL/min (for columns with dimension 50×150 mm). Mobile phase: gradient mode A: Water+0.5% formic acid (acidic conditions) A: Water+0.5% ammonium hydroxide solution (25%) (basic conditions) B: Acetonitrile Gradient: variable, e.g. for 75 mL/min: “extremely polar”: t[min] % A % B Flow mL/min: 0.000 100 0 75; 1.000 100 0 75; 3.500 80 20 75; 4.000 5 95 75; 6.000 5 95 75; 6.200 100 0 75; 6.600 100 0 75. “very polar”: t[min] % A % B Flow mL/min: 0.000 95 5 75; 0.100 95 5 75; 3.000 50 50 75; 4.000 5 95 75; 6.000 5 95 75; 6.200 95 5 75; 6.600 95 5 75; “polar”: t[min] % A % B Flow mL/min: 0.000 90 10 75; 0.010 90 10 75; 4.000 5 95 75; 6.000 5 95 75; 6.200 90 10 75; 6.600 90 10 75; “normal”: t[min] % A % B Flow mL/min: 0.000 80 20 75; 0.010 80 20 75; 4.000 5 95 75; 6.000 5 95 75; 6.200 80 20 75; 6.600 80 20 75; “lipophilic”: t[min] % A % B Flow mL/min: 0.000 70 30 75; 0.010 70 30 75; 3.500 5 95 75; 6.000 5 95 75; 6.200 70 30 75; 6.600 70 30 75; “very lipophilic”: t[min] % A % B Flow mL/min: 0.000 50 50 75; 0.010 50 50 75; 3.000 5 95 75; 6.000 5 95 75; 6.200 50 50 75; 6.600 50 50 75. Injection volume: 100-2500 μL. Collection: UV/MS/ELSD if available, and all possible combinations; Make-up flow rate: 0.50 mL/min. Make-up eluent MS: acetonitrile/water/TFA 70:30:0.025 (V/V/V);

MS ionization mode: ESI+.

LC-MS-Conditions:

Basic conditions: Column: Waters BEH C18, 3.0×50 mm, 2.5 μm/01593635616710; Temperature: 40° C.; Injection volume: 0.30 μl; Eluent A: water/NH₃ with c(NH₃)=13 mmol/I; Eluent B: Acetonitrile; Ionisation: ESI+; Gradient: at 0.0 min=5% B, at 0.01 min=5% B, at 1.20 min=95% B, at 1.90 min=95% B, at 2.00 min=5% B; Flow=1.6 mL/min.

Acidic conditions: Column: Zorbax RRHD SB-Aq, 2.1×50 mm, 1.8 μm/USEAF01579; Temperature: 40° C.; Injection volume: 0.15 μl; Eluent A: water 0.04% TFA; Eluent B: Acetonitrile; Ionisation: ESI+; Gradient: at 0.0 min=5% B, at 0.01 min=5% B, at 1.20 min=95% B, at 1.90 min=95% B, at 2.10 min=5% B; Flow=0.8 mL/min.

QC conditions: Column: Acquity UPLC CSH C18 1.7 μm 2.1×50 mm; Temperature: 60° C.; Injection volume: 0.25 μl, partial loop 2 μl; Eluent A: H2O+0.05% v/v Formic Acid; Eluent B: Acetonitrile+0.045% v/v Formic Acid; Gradient: 2% B to 98% B over 2.0 min; Flow=1.0 mL/min. Detection: UV at 214 nm and MS (Xevo Triple Quadrupole Detector Instrument); Ionisation: ESI+.

### Abbreviations (as Used Hereinbefore or Hereinafter)

Ac acetate
aq. aqueous
ACN acetonitrile
AlBN azobisisobutyronitrile
BPDS bathophenanthrolinedisulfonic acid disodium salt hydrate
BRP back pressure regulator
Boc tert-butyloxycarbonyl
CCl₄ carbon tetrachloride
CuAAC copper-catalyzed azide-alkyne cycloaddition
dba dibenzylideneacetone
DCM dichloromethane
DEA diethylamine
DIBALH diisobutylaluminium hydride
DIPEA diisopropyl ethyl amine (Hunig's base)
DMF dimethyl formamide
DMSO dimethylsulfoxide
dppf 1,1′-bis(diphenylphosphino)ferrocene
Et ethyl
EtOAc ethyl acetate
Et₂O diethyl ether
EtOH ethanol
FC flash chromatography
FGI functional group interconversion
h hour(s)
HATU (1-[Bis(dimethylamino)methylene]-1H-1,2,3-triazolo[4,5-b]pyridinium 3-oxid hexafluorophosphate)
LC-MS liquid chromatography coupled with mass spectrometry
Me methyl
MeOH methanol
MeCN acetonitrile
min. minute(s)
mL milliliters
NaBH₄ sodium borohydride
NBS N-bromo succinimide
n-Bu n-butyl
org. organic
Pd(II)dppf [1,1′-Bis(diphenylphosphino)ferrocene]dichloropalladium(II)
prepHPLC preparative HPLC
RT room temperature
rflx reflux
sat. saturated
SFC supercritical fluid chromatography
TFA trifluoroacetic acid
THF tetrahydrofuran
t_{R} HPLC retention time in minutes

### INTERMEDIATES SYNTHESIS

### Intermediate A: rac-1-(6-cyclopropylimidazo[1,5-a]pyridin-5-yl)prop-2-yn-1-ol

### Step 1: Preparation of ethyl 3,6-dichloropicolinate

To a pale yellow solution of 3,6-dichloropyridine-2-carboxylic acid (9701 mg, 48 mmol) in EtOH (42 mL) is added dropwise thionyl chloride (8.84 mL, 120 mmol) at 0° C. The resulting milky suspension is refluxed for 1 h to afford completion. The solvent is evaporated under reduced pressure. To the mixture is added saturated NaHCO₃, the pH is adjusted to 7 and the mixture is extracted with Et₂O. The combined organic layers are washed with brine, dried over MgSO₄, filtered and concentrated under reduced pressure. The pale green oil is dissolved in Et₂O and treated with activated charcoal for 10 min then filtered and concentrated under reduced pressure to give 11.4 g of ethyl 3,6-dichloropicolinate as a colorless oil. LCMS (acidic): t_{R}=0.86 min, [M+H]⁺=220.08.

### Step 2: Preparation of ethyl 3-chloro-6-cyanopicolinate

To a degassed solution of ethyl 3,6-dichloropicolinate (2403 mg, 10.9 mmol) in DMF (47 mL) are added zinc cyanide (1374 mg, 11.5 mmol), tris(dibenzylideneacetone)dipalladium(0) (619 mg, 0.655 mmol) and 1,1′-bis(diphenylphosphino)ferrocene (371 mg, 0.655 mmol). The resulting black suspension is stirred at 110° C. for 2 h30 then at RT overnight. More zinc cyanide 98% (65.4 mg, 0.546 mmol), tris(dibenzylideneacetone)dipalladium(0) (103 mg, 0.109 mmol) and 1,1′-bis(diphenylphosphino)ferrocene (61.8 mg, 0.109 mmol) are added and the reaction mixture is heated to 110° C. for 2 h30 to afford near completion. The mixture is cooled down to RT then concentrated under reduced pressure. The residue is redissolevd in Et₂O, filtered and concentrated under reduced pressure. The residue is purified by FC (Silica gel; EtOAc/Heptane) to give 1.44 g of ethyl 3-chloro-6-cyanopicolinate as a yellow oil. LCMS (acidic): t_{R}=0.82 min, [M+H]⁺=211.14.

### Step 3: Preparation of ethyl 6-(((tert-butoxycarbonyl)amino)methyl)-3-chloropicolinate

Ethyl 3-chloro-6-cyanopicolinate (1440 mg, 6.63 mmol) is dissolved in EtOH (70 mL) and di-tert-butyl dicarbonate (4431 mg, 19.9 mmol) is added. The reaction is conducted in the HCube-Pro with Ra—Ni catalyst (7 cm long) with the following conditions: T=70° C., P=10 bar, F=1.0 mL/min, 100% H₂ mode (1 pass). The mixture is concentrated under reduced pressure. The residue is purified by FC (Silica gel; EtOAc/Heptane) to give 3.89 g of ethyl 6-(((tert-butoxycarbonyl)amino)methyl)-3-chloropicolinate as a white solid. LCMS (acidic): t_{R}=0.91 min, [M+H]⁺=315.25.

### Step 4: Preparation of ethyl 3-chloro-6-(formamidomethyl)picolinate

Ethyl 6-(((tert-butoxycarbonyl)amino)methyl)-3-chloropicolinate (2840 mg, 9.02 mmol) is dissolved in trifluoroacetic acid (9 mL, 116 mmol). The mixture is stirred at RT for 30 min and concentrated under reduced pressure. The residue is dissolved in sat. aq. NaHCO₃ and the pH was adjusted to 8 by adding solid NaHCO₃. DCM (9 mL) is added and the mixture is stirred vigorously. A pre-heated (at 50° C. for 30 min) mixture of formic acid (2.4 mL, 62.4 mmol) and acetic anhydride (2.4 mL, 25.2 mmol) is added. The resulting mixture is stirred at RT overnight. The layers are separated and the aqueous layer extracted DCM (3×). The combined organic extracts are dried over MgSO₄, filtered and concentrated under reduced pressure. The resulting crude yellow oil is crystallized in DCM/Et₂O/Pentane to give 1.87 g of ethyl 3-chloro-6-(formamidomethyl)picolinate as a white solid. LCMS (acidic): t_{R}=0.64 min, [M+H]⁺=243.03.

### Step 5: Preparation of ethyl 6-chloroimidazo[1,5-a]pyridine-5-carboxylate

Ethyl 3-chloro-6-(formamidomethyl)picolinate (1875 mg, 7.73 mmol) is dissolved in toluene (40 mL). POCl₃ (1.44 mL, 15.5 mmol) is added at 0° C. and the mixture heated to 110° C. for 10 min. The mixture is concentrated under reduced pressure. The residue is redissolved in DCM and sat. aq. NaHCO₃ is added. The aqueous layer was extracted DCM (3×). The combined organic extracts are dried (MgSO₄), filtered and concentrated under reduced pressure. The crude red oil is purified by FC(Silica gel; EtOAc/Heptane) to give 1.613 g of ethyl 6-chloroimidazo[1,5-a]pyridine-5-carboxylate as a bright yellow oil, which solidified at RT. LCMS (basic): t_{R}=0.83 min, [M+H]⁺=225.13.

### Step 6: Preparation of (6-chloroimidazo[1,5-a]pyridin-5-yl)methanol

To an ice-chilled bright yellow solution of ethyl 6-chloroimidazo[1,5-a]pyridine-5-carboxylate (1613 mg, 7.18 mmol) in EtOH (92 mL) is added NaBH₄ (823 mg, 21.5 mmol). The resulting orange suspension is stirred at RT for 20 h to afford completion. EtOH is removed under reduced pressure, water is added and the mixture extracted with DCM. The combined org. layers are dried (MgSO₄), filtered and concentrated under reduced pressure. The crude residue is triturated in Et₂O/Pentane and filtered to give 1.0 g of (6-chloroimidazo[1,5-a]pyridin-5-yl)methanol as an off-white solid. LCMS (basic): t_{R}=0.56 min, [M+H]⁺=183.24.

### Step 7: Preparation of 6-chloroimidazo[1,5-a]pyridine-5-carbaldehyde

To a suspension of (6-chloroimidazo[1,5-a]pyridin-5-yl)methanol (1000 mg, 5.48 mmol) in DCM (30 mL) is added a suspension of Dess-Martin periodinane (3667 mg, 8.21 mmol) in DCM (20 mL) under an N₂ atmosphere at 0° C. The yellow suspension is stirred at 0° C. and then warmed up to RT for 2 h. Saturated solutions of aqueous NaHCO₃ and Na₂S₂O₃ are added and the mixture is extracted with DCM (3×). The organic extracts are washed with brine, dried over MgSO₄, filtered and concentrated under reduced pressure to yield 869 mg of 6-chloroimidazo[1,5-a]pyridine-5-carbaldehyde as a red solid. LCMS (acidic): t_{R}=0.65 min, [M+H]⁺=181.26.

### Step 8: Preparation of 6-cyclopropylimidazo[1,5-a]pyridine-5-carbaldehyde

A degassed mixture of 6-chloroimidazo[1,5-a]pyridine-5-carbaldehyde (88.5 mg, 0.49 mmol), cyclopropylboronic acid (126 mg, 1.47 mmol), tricyclohexylphosphine (41.2 mg, 0.147 mmol), palladium(II) acetate (11.2 mg, 0.049 mmol) and K₂CO₃ (135 mg, 0.98 mmol) in toluene (8.5 mL) and water (3.4 mL) is heated at 80° C. overnight (half conversion). More cyclopropylboronic acid (378 mg, 4.4 mmol), tricyclohexylphosphine (185 mg, 0.66 mmol) and palladium(II) acetate (50.4 mg, 0.22 mmol) are added and the mixture was heated to 100° C. for 3 h to afford completion. The mixture is cooled to RT, diluted with EtOAc and filtered through a short pad of celite. The layers are separated and the aqueous layer extracted twice more with EtOAc. The combined organic extracts are dried over MgSO₄, filtered and concentrated under reduced pressure. The brown residue is dissolved in MeCN and successively washed with heptane and pentane, then concentrated under reduced pressure. The brown oil is triturated in Et₂O/Pentane and the resulting precipitate is collected by filtration to give 654 mg of 6-cyclopropylimidazo[1,5-a]pyridine-5-carbaldehyde as a beige solid. LCMS (acidic): t_{R}=0.47 min, [M+H]⁺=187.33.

### Step 9: Preparation of rac-1-(6-cyclopropylimidazo[1,5-a]pyridin-5-yl)prop-2-yn-1-ol (Intermediate A)

A solution of 6-cyclopropylimidazo[1,5-a]pyridine-5-carbaldehyde (654 mg, 3.51 mmol) in THF (25 mL) and Et₂O (10 mL) is cooled to −10° C. and treated with ethynylmagnesium bromide solution 0.5 M in THF (9 mL, 4.5 mmol). The reaction mixture is stirred at −10° C. and warmed up to RT for 4 h to afford completion. Ice and sat. aq. NH₄Cl are added and the mixture extracted with EtOAc (3×). The combined org. layers are dried over MgSO₄, filtered and concentrated under reduced pressure to give 701 mg of rac-1-(6-cyclopropylimidazo[1,5-a]pyridin-5-yl)prop-2-yn-1-ol as a brown foam. LCMS (acidic): t_{R}=0.52 min, [M+H]⁺=213.16.

### Intermediate B: rac-1-(6-cyclopropylimidazo[1,5-a]pyrazin-5-yl)prop-2-yn-1-ol

### Step 1: Preparation of methyl 3-chloro-6-methylpyrazine-2-carboxylate

Methyl 6-bromo-3-chloropyrazine-2-carboxylate (13.156 g, 49.7 mmol), trimethylboroxine (7.02 mL, 49.7 mmol), K₂CO₃ (13.738 g, 99.4 mmol) and Pd(II)dppf (2.029 g, 2.48 mmol) are suspended in dioxane (158 mL). The mixture is degassed with N₂ for 10 min and heated at 100° C. for 36 h. The mixture is cooled to RT and filtered through a pad of celite. The filtrate is concentrated under reduced pressure. The crude product is purified by FC (Silica gel; EtOAc/Heptane) to give 7.7 g of methyl 3-chloro-6-methylpyrazine-2-carboxylate as a yellow oil. LCMS (acidic): t_{R}=0.67 min, [M+H]⁺=187.18.

### Step 2: Preparation of methyl 6-(bromomethyl)-3-chloropyrazine-2-carboxylate

Methyl 3-chloro-6-methylpyrazine-2-carboxylate (5.84 g, 29.7 mmol) is dissolved in CCl₄ (82 mL). NBS (8.018 g, 44.6 mmol) and AIBN (249 mg, 1.49 mmol) are sequentially added. The mixture is refluxed for 24 h. More AlBN is added and the mixture stirred at reflux until almost completion of the reaction. The mixture is cooled to RT and concentrated under reduced pressure. To the residue is added water and EtOAc, the layers are separated and the aqueous phase is further extracted with EtOAc (2×). The combined organic layers are dried (MgSO₄), filtered and concentrated under reduced pressure. The crude product is purified by FC (Silica gel; EtOAc/Heptane) to give 3.57 g of methyl 6-(bromomethyl)-3-chloropyrazine-2-carboxylate as a light yellow oil. LCMS (acidic): t_{R}=0.79 min, [M+H]⁺=no mass.

### Step 3: Preparation of methyl 3-chloro-6-(formamidomethyl)pyrazine-2-carboxylate

To a solution of methyl 6-(bromomethyl)-3-chloropyrazine-2-carboxylate (8790 mg, 33.1 mmol) in DMF (116 mL) is added sodium diformylamide (3568 mg, 36.4 mmol). The reaction is stirred at RT for 1 h. A sat. aq. solution of NaHCO₃ is added and the reaction mixture stirred at RT overnight until complete conversion into the desired product. EtOAc is added, the layers separated and the aq. layer extracted with EtOAc (3×). The combined org. extracts are washed with brine, dried (MgSO₄), filtered and concentrated under reduced pressure to give 7.5 g of methyl 3-chloro-6-(formamidomethyl)pyrazine-2-carboxylate as a black oil. LC-MS (acidic): t_{R}=0.52 min, [M+H]⁺=230.23.

### Step 4: Preparation of methyl 6-chloroimidazo[1,5-a]pyrazine-5-carboxylate

Methyl 3-chloro-6-(formamidomethyl)pyrazine-2-carboxylate (3.077 g, 13.4 mmol) is dissolved in toluene (24 mL). POCl₃ (2.5 mL, 26.8 mmol) is added and the mixture is heated at 70° C. for 1 h. Aq. NaHCO₃ is added to the mixture until pH=7. The product is extracted with EtOAc (3×). The combined organic extracts are dried (MgSO₄), filtered and concentrated under reduced pressure. The crude product is purified by FC (Silica gel; EtOAc/Heptane) to give 1.82 g of methyl 6-chloroimidazo[1,5-a]pyrazine-5-carboxylate as a brown solid. LC-MS (acidic): t_{R}=0.64 min, [M+H]⁺=212.06.

### Step 5: Preparation of methyl 6-cyclopropylimidazo[1,5-a]pyrazine-5-carboxylate

A mixture of methyl 6-chloroimidazo[1,5-a]pyrazine-5-carboxylate (735 mg, 3.47 mmol), cyclopropylzinc bromide solution 0.5 M in THF (10.4 mL, 5.21 mmol) and tetrakis(triphenylphosphine)palladium(0) (40.2 mg, 0.035 mmol) in THF (7 mL) is stirred under N₂ for 1 h 15 at 70° C. Sat. aq. NaHCO₃ is added, the mixture diluted with EtOAc and filtered. The layers are separated and the aqueous phase is extracted with EtOAc (2×). The combined organic extracts are dried (MgSO₄), filtered and concentrated under reduced pressure. The crude product is purified by FC (Silica gel; Heptane/EtOAc) to give 262 mg of methyl 6-cyclopropylimidazo[1,5-a]pyrazine-5-carboxylate as a yellow solid. LC-MS (acidic): t_{R}=0.70 min, [M+H]⁺=218.15.

### Step 6: Preparation of 6-cyclopropyl-N-methoxy-N-methylimidazo[1,5-a]pyrazine-5-carboxamide

Step 6.1: Saponification: methyl 6-cyclopropylimidazo[1,5-a]pyrazine-5-carboxylate (524 mg, 2.41 mmol) is dissolved in THF (7.7 mL) and water (3.85 mL). Lithiumhydroxide monohydrate (123 mg, 2.89 mmol) is added and the mixture is stirred at RT for 2 h 15. The mixture is concentrated under reduced pressure.

Step 6.2: Amide coupling: The residue is dissolved in DMF (10 mL). DIPEA (1.24 mL, 7.24 mmol), N,O-dimethylhydroxylamine hydrochloride (288 mg, 2.89 mmol) and HATU (1.101 g, 2.89 mmol) are added and the mixture is stirred at RT for 2 h 30 The mixture is concentrated under reduced pressure. The crude product is purified by preparative HPLC (basic conditions) to give 374 mg of 6-cyclopropyl-N-methoxy-N-methylimidazo[1,5-a]pyrazine-5-carboxamide as a yellow solid. LC-MS (acidic): t_{R}=0.60 min, [M+H]⁺=247.14.

### Step 7: Preparation of 6-cyclopropylimidazo[1,5-a]pyrazine-5-carbaldehyde

To an ice-cold solution of 6-cyclopropyl-N-methoxy-N-methylimidazo[1,5-a]pyrazine-5-carboxamide (323 mg, 1.31 mmol) in THF (8.5 mL) is added diisobutylaluminium hydride solution (1.0 M in toluene, 1.31 mL, 1.31 mmol) in a dropwise manner. The resulting solution is stirred at 0° C. for 30 min. More diisobutylaluminium hydride solution (1.0 M in toluene, 0.66 mL, 0.66 mmol) is added at 0° C. and the mixture is stirred for 1 h at this temperature. Sat. aq. NH₄Cl is added, and the mixture is extracted with EtOAc (3×). The combined organic extracts are dried (MgSO₄), filtered and concentrated under reduced pressure to give 252 mg of 6-cyclopropylimidazo[1,5-a]pyrazine-5-carbaldehyde as a yellow solid. LC-MS (basic): t_{R}=0.63 min, [M+H]⁺=188.29. ¹H NMR (500 MHz, DMSO) δ: 10.68 (s, 1H), 9.49 (s, 1H), 9.28 (s, 1H), 8.08 (d, 1H), 3.00 (m, 1H), 1.24-1.25 (m, 2H), 1.12 (dd, J1=8.0 Hz, J2=2.9 Hz, 2H).

### Step 8: Preparation of rac-1-(6-cyclopropylimidazo[1,5-a]pyrazin-5-yl)prop-2-yn-1-ol (Intermediate B)

6-Cyclopropylimidazo[1,5-a]pyrazine-5-carbaldehyde (245 mg, 1.31 mmol) is dissolved in THF (5.8 mL). The solution is cooled to 0° C. and ethynylmagnesium bromide solution 0.5 M in THF (7.86 mL, 3.93 mmol) is added dropwise. The reaction mixture is stirred at 0° C. for 1 h. Aq. NH₄Cl is added and the product is extracted with EtOAc (3×). The combined organic extracts are dried (MgSO₄), filtered and concentrated under reduced pressure to give 285 mg of rac-1-(6-cyclopropylimidazo[1,5-a]pyrazin-5-yl)prop-2-yn-1-ol as a yellow-white solid. LC-MS (basic): t_{R}=0.58 min, [M+H]⁺=214.27.

### Intermediate Ba: (S)-1-(6-cyclopropylimidazo[1,5-a]pyrazin-5-yl)prop-2-yn-1-ol

Separation of rac-1-(6-cyclopropylimidazo[1,5-a]pyrazin-5-yl)prop-2-yn-1-ol on chiral stationary phase:

Column: ChiralPak AS-H, 30×250 mm, 5 μm; Temperature: 40° C.; BPR: 100 bar; Detector Wavelength: 227 nm; Mobile Phase: ACN/EtOH/DEA 50:50:0.1; Flow: 160.00 mL/min; Injection Volume: 6 mL.

613 mg of the racemate are separated by the method described above to give 312 mg of the S-enantiomer and 350 mg of the R-enantiomer.

### Intermediate C: rac-1-(6-chloroimidazo[1,5-a]pyridin-5-yl)prop-2-yn-1-ol

An ice-chilled solution of 6-chloroimidazo[1,5-a]pyridine-5-carbaldehyde—Intermediate A, step 7—(73.9 mg, 0.409 mmol) in THF (1.6 mL) is treated with ethynylmagnesium bromide solution 0.5 M in THF (2.46 mL, 17.9 mmol). The reaction mixture is stirred at 0-10° C. for 2 h (until completion of the reaction) then water and aq. NH₄Cl are added. The mixture is extracted with DCM (3×), the combined organic extracts dried over MgSO₄, filtered and concentrated under reduced pressure to give 77.8 mg of rac-1-(6-chloroimidazo[1,5-a]pyridin-5-yl)prop-2-yn-1-ol as an orange solid. LC-MS (basic): t_{R}=0.65 min, [M+H]⁺=207.19.

### Intermediates 3a and 3b: (S)-1-(6-chloroimidazo[1,5-a]pyridin-5-yl)prop-2-yn-1-ol and (R)-1-(6-chloroimidazo[1,5-a]pyridin-5-yl)prop-2-yn-1-ol

Separation of rac-1-(6-chloroimidazo[1,5-a]pyridin-5-yl)prop-2-yn-1-ol on chiral stationary phase:

Column: ChiralPak IH, 30×250 mm, 5 μm; Temperature: 40° C.; BPR: 100 bar; Detector Wavelength: 225 nm; Mobile Phase: 25% EtOH and 75% CO₂; Flow: 160.00 mL/min; Injection Volume: 1 mL.

233 mg of the racemate are separated by the method described above to give 100 mg of the S-enantiomer and 114 mg of the R-enantiomer.

### Intermediate D: rac-1-(6-methylimidazo[1,5-a]pyridin-5-yl)prop-2-yn-1-ol

### Step 1: Preparation of 6-methylimidazo[1,5-a]pyridine-5-carbaldehyde

A degassed suspension of 6-chloroimidazo[1,5-a]pyridine-5-carbaldehyde—Intermediate A, step 7—(117 mg, 0.648 mmol), trimethylboroxine (0.453 mL, 3.24 mmol), K₂CO₃ (181 mg, 1.3 mmol), [1,1′-bis(diphenylphosphino)ferrocene]clichloropalladium(11), complex with dichloromethane (26.5 mg, 0.0324 mmol) in dioxane (2 mL) is heated at 110° C. for 1 h to afford completion. The mixture is cooled to RT, diluted with EtOAc and filtered through a pad of celite. The filtrate is concentrated under reduced pressure, the residual brown oil triturated in Et₂O/pentane and filtered to afford 0.114 g of 6-methylimidazo[1,5-a]pyridine-5-carbaldehyde as an orange solid. LC-MS (basic): t_{R}=0.57 min, [M+H]⁺=161.14.

### Step 2: rac-1-(6-methylimidazo[1,5-a]pyridin-5-yl)prop-2-yn-1-ol (Intermediate D)

An ice-chilled solution of 6-methylimidazo[1,5-a]pyridine-5-carbaldehyde (60.9 mg, 0.38 mmol) in THF (1.6 mL) is treated with ethynylmagnesium bromide solution 0.5 M in THF (2.28 mL, 1.14 mmol). The reaction mixture is stirred at 0-10° C. for 2 h (until completion of the reaction). Water and aq. NH₄Cl are added and the mixture extracted with DCM (3×). The combined organic layers are dried over MgSO₄, filtered and concentrated under reduced pressure to give 70 mg of rac-1-(6-methylimidazo[1,5-a]pyridin-5-yl)prop-2-yn-1-ol as a brown solid. LC-MS (acidic): t_{R}=0.45 min, [M+H]⁺=187.21.

### Intermediate E: rac-1-(6-ethylimidazo[1,5-a]pyridin-5-yl)prop-2-yn-1-ol

### Step 1: Preparation of ethyl 6-ethylimidazo[1,5-a]pyridine-5-carboxylate

A degassed bright yellow solution of ethyl 6-chloroimidazo[1,5-a]pyridine-5-carboxylate—Intermediate A, step 5—(679 mg, 3.02 mmol) in THF (7.2 mL) is cooled down to 0° C. then [1,3-bis(diphenylphosphino)propane]clichloronickel(II) (82 mg, 0.151 mmol) is added followed by a degassed solution of ethylmagnesium bromide solution 1.0 M in THF (4.53 mL, 4.53 mmol). The reaction mixture is stirred at 0° C. for 30 min then heated to 70° C. for 1 h. Water is added followed by sat. aq. NaHCO₃. The product is extracted with EtOAc (3×). The combined organic extracts are dried (MgSO₄), filtered and concentrated under reduced pressure. The crude residue is purified by preparative HPLC (basic conditions) to give 0.380 g of ethyl 6-ethylimidazo[1,5-a]pyridine-5-carboxylate as a yellow oil. LC-MS (acidic): t_{R}=0.60 min, [M+H]⁺=219.30.

### Step 2: Preparation of (6-ethylimidazo[1,5-a]pyridin-5-yl)methanol

To an ice-chilled solution of ethyl 6-ethylimidazo[1,5-a]pyridine-5-carboxylate (380 mg, 1.74 mmol) in EtOH (12 mL) and DCM (2 mL) is added NaBH₄ (200 mg, 5.22 mmol) portionwise. The mixture is stirred at RT overnight. More NaBH₄ is added and the mixture stirred at RT until completion of the reaction. EtOH is removed under reduced pressure, water is added and the mixture extracted 3 times with DCM. The combined org. extracts are dried over MgSO₄, filtered and concentrated under reduced pressure to give 0.320 g of (6-ethylimidazo[1,5-a]pyridin-5-yl)methanol as a pale yellow foam. LC-MS (acidic): t_{R}=0.43 min, [M+H]⁺=177.40.

### Step 3: Preparation of 6-ethylimidazo[1,5-a]pyridine-5-carbaldehyde

A solution of (6-ethylimidazo[1,5-a]pyridin-5-yl)methanol (298 mg, 1.69 mmol) in CH₃CN/DCM 1:1 (6 mL) is treated with MnO₂ (817 mg, 8.46 mmol) ad RT and heated to 70° C. under microwave radiations for 1 h. More MnO₂ (817 mg, 8.46 mmol) is added and the mixture further stirred at 70° C. under microwave radiations for 45 min. The suspension is filtered and the filter cake rinsed with DCM. The filtrate is concentrated under reduced pressure, the residue suspended in Et₂O, filtered and the filtrate concentrated under reduced pressure to give 0.233 g of 6-ethylimidazo[1,5-a]pyridine-5-carbaldehyde as a brown oil. LC-MS (basic): t_{R}=0.65 min, [M+H]⁺=175.24.

### Step 4: Preparation of rac-1-(6-ethylimidazo[1,5-a]pyridin-5-yl)prop-2-yn-1-ol (Intermediate E)

A solution of 6-ethylimidazo[1,5-a]pyridine-5-carbaldehyde (256 mg, 1.47 mmol) in THF (10 mL) and Et₂O (4 mL) is cooled to 0° C. and treated with dropwise addition of ethynylmagnesium bromide solution 0.5 M in THF (3.8 mL, 1.91 mmol) over 20 min. The reaction mixture is stirred at 0° C. for 1 h and at RT overnight. More ethynylmagnesium bromide solution 0.5 M in THF is added and the mixture further stirred until completion of the reaction. Saturated aqueous NH₄Cl is added and the product is extracted three times with EtOAc. The combined organic extracts are dried over MgSO₄, filtered and concentrated under reduced pressure. The crude residue is purified by FC (Silica gel; Heptane/EtOAc) to give 0.060 g of rac-1-(6-ethylimidazo[1,5-a]pyridin-5-yl)prop-2-yn-1-ol as a brown solid. LC-MS (acidic): t_{R}=0.50 min, [M+H]⁺=201.28.

### Intermediate F: rac-1-(6-ethylimidazo[1,5-a]pyrazin-5-yl)prop-2-yn-1-ol

### Step 1: Preparation of methyl 6-vinylimidazo[1,5-a]pyrazine-5-carboxylate

To a solution of methyl 6-chloroimidazo[1,5-a]pyrazine-5-carboxylate—Intermediate B, step 4—(200 mg, 0.93 mmol) in EtOH (4 mL) are added potasssium vinyltrifluoroborate (144 mg, 1.02 mmol) and Et₃N (0.19 mL, 1.39 mmol) at RT and the mixture is stirred for 5 min. [1,1′-Bis(diphenylphosphino)ferrocene]clichloropalladium(II) (68 mg, 0.09 mmol) is then added and the mixture degassed with N₂ for 5 min. The reaction mixture is heated at 90° C. under microwave irradations for 2 h and concentrated under reduced pressure. The residue is diluted with water and EtOAc, filtered, the layers separated and the aqueous layer extracted with EtOAc (2×). The combined organic extracts are dried (MgSO₄), filtered and concentrated under reduced pressure. The crude residue is purified by FC (silica gel, Het/EtOAc) to give 132 mg of methyl 6-vinylimidazo[1,5-a]pyrazine-5-carboxylate as a yellow solid. LC-MS (acidic): t_{R}=0.65 min, [M+H]⁺=204.26.

### Step 2: Preparation of methyl 6-ethylimidazo[1,5-a]pyrazine-5-carboxylate

To a degassed (3 vacuum/N₂ cycles) solution of methyl 6-vinylimidazo[1,5-a]pyrazine-5-carboxylate (132 mg, 0.65 mmol) in MeOH (12 mL) is added at 0° C. Pd on charcoal (10% Pd, 69 mg, 0.06 mmol). The resulting black suspension is stirred at 0° C. under a H₂ atmosphere for 10 min. The mixture is filtered through a Whatman 0.45 uM glass microfiber filter and washed with MeOH. The filtrate is concentrated under reduced pressure to give 106 mg of methyl 6-ethylimidazo[1,5-a]pyrazine-5-carboxylate as a yellow sticky oil. LC-MS (acidic): t_{R}=0.58 min, [M+H]⁺=206.26.

### Step 3: Preparation of 6-ethyl-N-methoxy-N-methylimidazo[1,5-a]pyrazine-5-carboxamide

To a solution of methyl 6-ethylimidazo[1,5-a]pyrazine-5-carboxylate (106 mg, 0.52 mmol) in THF (1.65 mL) and water (0.83 mL) is added LiOH (26 mg, 0.62 mmol) and the mixture stirred at RT For 1 h. The mixture is then concentrated under reduced pressure and the residue redissolved in DMF (2.6 mL). DIPEA (0.26 mL, 1.55 mmol), N,O-dimethylhydroxylamine hydrochloride (62 mg, 0.62 mmol) and HATU (236 mg, 0.62 mmol) are added. The reaction mixture is stirred at RT for 16 h. The mixture is filtered and washed with DMF and the filtrate concentrated under reduced pressure. The crude residue is purified by preparative HPLC (basic conditions) to give 80 mg of 6-ethyl-N-methoxy-N-methylimidazo[1,5-a]pyrazine-5-carboxamide. LC-MS (acidic): t_{R}=0.54 min, [M+H]⁺=234.82.

### Step 4: Preparation of 6-ethylimidazo[1,5-a]pyrazine-5-carbaldehyde

To an ice-cold solution of 6-ethyl-N-methoxy-N-methylimidazo[1,5-a]pyrazine-5-carboxamide (80 mg, 0.34 mmol) in THF (2.2 mL) is added diisobutylaluminium hydride solution (1.0 M in toluene, 0.34 mL, 0.34 mmol) in a dropwise manner. The resulting solution is stirred at 0° C. for 30 min. More diisobutylaluminium hydride solution (1.0 M in toluene, 0.34 mL, 0.34 mmol) is added at 0° C. and the mixture is stirred for 1 h at this temperature. Sat. aq. NH₄Cl is added, and the mixture is extracted with EtOAc (3×). The combined organic extracts are dried (MgSO₄), filtered and concentrated under reduced pressure to give 59 mg of 6-ethylimidazo[1,5-a]pyrazine-5-carbaldehyde as a dark yellow sticky oil. LC-MS (basic): t_{R}=0.51 min, [M+H]⁺=176.14.

### Step 5: Preparation of rac-1-(6-ethylimidazo[1,5-a]pyrazin-5-yl)prop-2-yn-1-ol (Intermediate F)

A solution of 6-ethylimidazo[1,5-a]pyrazine-5-carbaldehyde (59 mg, 0.34 mmol) in THF (1.5 mL) is cooled to 0° C. and treated with dropwise addition of ethynylmagnesium bromide solution 0.5 M in THF (2.02 mL, 1.01 mmol) over 20 min. The reaction mixture is stirred at 0° C. for 1 h and 15 min. Saturated aqueous NH₄Cl is added and the product is extracted three times with EtOAc. The combined organic extracts are dried over MgSO₄, filtered and concentrated under reduced pressure to give 63 mg of rac-1-(6-ethylimidazo[1,5-a]pyrazin-5-yl)prop-2-yn-1-ol as a yellow oil. LC-MS (basic): t_{R}=0.49 min, [M+H]⁺=202.18.

### Intermediate G: 6-chloro-3-(ethylthio)imidazo[1,5-a]pyridine

### Step 1: Preparation of 6-chloroimidazo[1,5-a]pyridine-3-thiol

To a solution of (5-chloropyridin-2-yl)methanamine dihydrochloride (431 mg, 2.00 mmol) in MeOH (11 mL) is added Et₃N (0.56 mL, 4.00 mmol) followed by carbon disulfide (0.84 mL, 14.0 mmol). The reaction mixture is stirred at reflux for 2 h30. The resulting dark orange solution is cooled down to RT and concentrated under reduced pressure. The residue is partitioned between water and CH₂Cl₂. The layers are separated and the aq layer extracted with CH₂Cl₂ (2×). The combined org extracts are dried (MgSO₄), filtered and concentrated under reduced pressure. The crude residue is triturated in MeOH/Et₂O/Petroleum ether and the solid collected by filtration to give 256 mg of 6-chloroimidazo[1,5-a]pyridine-3-thiol as a beige solid. LC-MS (acidic): t_{R}=0.63 min, [M+H]⁺=185.16.

### Step 2: Preparation of 6-chloro-3-(ethylthio)imidazo[1,5-a]pyridine (Intermediate G)

A suspension of 6-chloroimidazo[1,5-a]pyridine-3-thiol (256 mg, 1.39 mmol), iodoethane (0.13 mL, 1.58 mmol) and K₂CO₃ (646 mg, 4.67 mmol) in acetone (12 mL) is stirred at 45° C. for 2 h. The mixture is then concentrated under reduced pressure and the residue partitioned between water and CH₂Cl₂. The layers are separated and the aq layer is extracted with CH₂Cl₂ (2×). The combined org extracts are dried (MgSO₄), filtered and concentrated under reduced pressure. The crude residue is triturated in CH₂Cl₂/Et₂O and the filtrate is concentrated under reduced pressure to give 203 mg of 6-chloro-3-(ethylthio)imidazo[1,5-a]pyridine as a brown solid. LC-MS (acidic): t_{R}=0.68 min, [M+H]⁺=213.18.

### EXAMPLES SYNTHESIS

### Example 1: rac-(6-Chloro-imidazo[1,5-a]pyridin-5-yl)-(1-phenyl-1H-[1,2,3]triazol-4-yl)-methanol

A degassed solution of Intermediate C, rac-1-(6-chloroimidazo[1,5-a]pyridin-5-yl)prop-2-yn-1-ol (38.8 mg, 0.188 mmol), azidobenzene solution ˜0.5 M in tert-butyl methyl ether (0.45 mL, 0.226 mmol), copper(II) sulfate pentahydrate (4.69 mg, 0.0188 mmol), L(+)-ascorbic acid sodium salt (7.52 mg, 0.0376 mmol) in DMF (1 mL) and water (0.2 mL) is stirred at RT overnight.

The mixture is filtered and purified by preparative HPLC (basic) to give 36.2 mg of rac-(6-Chloro-imidazo[1,5-a]pyridin-5-yl)-(1-phenyl-1H-[1,2,3]triazol-4-yl)-methanol as an off-white solid. LC-MS (QC): t_{R}=0.659 min, [M+H]⁺=326.1. ¹H NMR (500 MHz, DMSO) δ: 9.10 (s, 1H), 8.62 (s, 1H), 7.93 (d, J=7.9 Hz, 2H), 7.66 (d, J=9.5 Hz, 1H), 7.59 (t, J=7.6 Hz, 2H), 7.49 (m, 2H), 7.02 (d, J=4.2 Hz, 1H), 6.91 (d, J=9.5 Hz, 1H), 6.82 (d, J=4.1 Hz, 1H).

### Example 1a: (R)-(6-Chloro-imidazo[1,5-a]pyridin-5-yl)-(1-phenyl-1H-[1,2,3]triazol-4-yl)-methanol

Separation of rac-(6-chloro-imidazo[1,5-a]pyridin-5-yl)-(1-phenyl-1H-[1,2,3]triazol-4-yl)-methanol on chiral stationary phase:

Column: ChiralPak AS-H 30×250 mm, 5 μM; Detector Wavelength: UV 226 nM; Eluent: 75% CO₂ and 25% EtOH+0.1% DEA; Flow: 160.00 mL/min; BPR: 100 bar; Temperature: 40° C. Injection volume: 2000 μl. 25 mg of the racemate are separated by the method described above to give 9 mg of the R-enantiomer and 9 mg of the S-enantiomer.

Example 1a: LC-MS (QC): t_{R}=0.659 min, [M+H]⁺=326.1.

### Example 2: rac-(6-Methyl-imidazo[1,5-a]pyridin-5-yl)-(1-phenyl-1H-[1,2,3]triazol-4-yl)-methanol

Prepared following the procedure described for Example 1 using Intermediate D, rac-1-(6-methylimidazo[1,5-a]pyridin-5-yl)prop-2-yn-1-ol and azidobenzene. Purification by preparative HPLC (basic conditions) gives rac-(6-methyl-imidazo[1,5-a]pyridin-5-yl)-(1-phenyl-1H-[1,2,3]triazol-4-yl)-methanol. LC-MS (QC): t_{R}=0.519 min, [M+H]⁺=306.2. ¹H NMR (500 MHz, DMSO) δ: 9.06 (d, 1H), 8.48-8.61 (m, 1H), 7.85-8.02 (m, 2H), 7.57-7.61 (m, 2H), 7.47-7.50 (m, 2H), 7.33 (s, 1H), 6.69-6.81 (m, 1H), 6.54-6.66 (m, 2H), 2.47 (m, 3H).

### Example 2a: (R)-(6-Methyl-imidazo[1,5-a]pyridin-5-yl)-(1-phenyl-1H-[1,2,3]triazol-4-yl)-methanol

Separation of rac-(6-methyl-imidazo[1,5-a]pyridin-5-yl)-(1-phenyl-1H-[1,2,3]triazol-4-yl)-methanol on chiral stationary phase:

Column: ChiralPak AS-H, 30×250 mm, 5 μm; Temperature: 40° C.; BPR: 100 bar; Detector Wavelength: 222 nm; Eluent: 75% CO₂ and 25% EtOH+0.1% DEA; Flow: 160.00 mL/min; Injection Volume: 2.5 mL.

25 mg of the racemate are separated by the method described above to give 9 mg of the R-enantiomer and 9 mg of the S-enantiomer.

Example 2a: LC-MS (QC): t_{R}=0.518 min, [M+H]⁺=306.2.

### Example 3: rac-(6-Cyclopropyl-imidazo[1,5-a]pyrazin-5-yl)-(1-phenyl-1H-[1,2,3]triazol-4-yl)-methanol

Prepared following the procedure described for Example 1 using Intermediate B, rac-1-(6-cyclopropylimidazo[1,5-a]pyrazin-5-yl)prop-2-yn-1-ol and azidobenzene. Purification by preparative HPLC (basic conditions) gives rac-(6-cyclopropyl-imidazo[1,5-a]pyrazin-5-yl)-(1-phenyl-1H-[1,2,3]triazol-4-yl)-methanol. LC-MS (QC): t_{R}=0.727 min, [M+H]⁺=333.2. ¹H NMR (500 MHz, DMSO) δ: 9.07 (d, 1H), 8.97 (s, 1H), 8.65 (s, 1H), 7.92-7.94 (m, 2H), 7.78 (s, 1H), 7.60 (m, 2H), 7.48-7.51 (m, 1H), 6.89 (d, J=3.5 Hz, 1H), 6.83 (d, J=3.8 Hz, 1H), 2.48 (m, 1H), 1.06-1.10 (m, 1H), 0.94-1.01 (m, 3H).

### Example 3a: (R)-(6-Cyclopropyl-imidazo[1,5-a]pyrazin-5-yl)-(1-phenyl-1H-[1,2,3]triazol-4-yl)-methanol

Separation of rac-(6-cyclopropyl-imidazo[1,5-a]pyrazin-5-yl)-(1-phenyl-1H-[1,2,3]triazol-4-yl)-methanol on chiral stationary phase:

Column: ChiralPak AS-H 30×250 mm, 5 μM; Detector Wavelength: UV 227 nM; Eluent: 75% CO₂ and 25% EtOH+0.1% DEA; Flow: 160.00 mL/min; BPR: 100 bar; Temperature: 40° C. Injection volume: 1900 μl.

19 mg of the racemate are separated by the method described above to give:

5 mg of the R-enantiomer Example 3a and 6 mg of the S-enantiomer.

Example 3a: LC-MS (QC): t_{R}=0.727 min, [M+H]⁺=333.2.

### Example 4: rac-(6-Cyclopropyl-imidazo[1,5-a]pyridin-5-yl)-[1-(4-methoxy-phenyl)-1H-[1,2,3]triazol-4-yl]-methanol

Prepared following the procedure described for Example 1 using Intermediate A, rac-1-(6-cyclopropylimidazo[1,5-a]pyridin-5-yl)prop-2-yn-1-ol and 1-azido-4-methoxybenzene. Purification by preparative HPLC (basic conditions) gives rac-(6-cyclopropyl-imidazo[1,5-a]pyridin-5-yl)-[1-(4-methoxy-phenyl)-1H-[1,2,3]triazol-4-yl]-methanol. LC-MS (QC): t_{R}=0.630 min, [M+H]⁺=362.2. ¹H NMR (500 MHz, DMSO) δ: 8.95 (s, 1H), 8.52 (s, 1H), 7.72-7.99 (m, 2H), 7.49 (d, J=9.4 Hz, 1H), 7.23-7.41 (m, 1H), 7.13 (m, 2H), 7.01 (d, J=3.6 Hz, 1H), 6.67 (m, 2H), 3.73-3.94 (m, 3H), 2.16-2.30 (m, 1H), 2.00-2.15 (m, 2H), 0.97-1.07 (m, 2H), 0.86-0.91 (m, 1H), 0.71-0.83 (m, 1H).

### Example 5: rac-(6-Cyclopropyl-imidazo[1,5-a]pyridin-5-yl)-(1-p-tolyl-1H-[1,2,3]triazol-4-yl)-methanol

Prepared following the procedure described for Example 1 using Intermediate A, rac-1-(6-cyclopropylimidazo[1,5-a]pyridin-5-yl)prop-2-yn-1-ol and azidotoluene. Purification by preparative HPLC (basic conditions) gives rac-(6-cyclopropyl-imidazo[1,5-a]pyridin-5-yl)-(1-p-tolyl-1H-[1,2,3]triazol-4-yl)-methanol. LC-MS (QC): t_{R}=0.678 min, [M+H]⁺=346.2. ¹H NMR (500 MHz, DMSO) δ: 8.97 (s, 1H), 8.48-8.51 (m, 1H), 7.80 (d, J=8.4 Hz, 2H), 7.48 (d, J=9.4 Hz, 1H), 7.39 (m, 2H), 7.32 (s, 1H), 6.95-7.08 (m, 1H), 6.67-6.71 (m, 1H), 6.65 (m, 1H), 2.38 (s, 3H), 2.11-2.31 (m, 1H), 0.95-1.13 (m, 2H), 0.83-0.92 (m, 1H), 0.74-0.83 (m, 1H).

### Example 6: rac-(4-{4-[(6-Cyclopropyl-imidazo[1,5-a]pyridin-5-yl)-hydroxy-methyl]-[1,2,3]triazol-1-yl}-phenylycarbamic acid methyl ester

### Step 1: Preparation of methyl (4-azidophenyl)carbamate

Prepared according to the procedure described for Example 7, step 1 using 4-methoxycarbonylaminophenylboronic acid.

### Step 2

Prepared following the procedure described for Example 1, and using Intermediate A, rac-1-(6-cyclopropylimidazo[1,5-a]pyridin-5-yl)prop-2-yn-1-ol and methyl (4-azidophenyl)carbamate. Purification by prepHPLC (basic conditions) to give rac-(4-{4-[(6-cyclopropyl-imidazo[1,5-a]pyridin-5-yl)-hydroxy-methyl]-[1,2,3]triazol-1-yl}-phenyl)-carbamic acid methyl ester. LC-MS (QC): t_{R}=0.589; [M+H]⁺=405.2.

### Example 7: rac-2-Chloro-4-{4-[(6-cyclopropyl-imidazo[1,5-a]pyridin-5-yl)-hydroxy-methyl]-[1,2,3]triazol-1-yl}-phenol

### Step 1: Preparation of 4-azido-2-chlorophenol

In a round-bottomed flask, (3-chloro-4-hydroxyphenyl)boronic acid (508 mg, 2.83 mmol), sodium azide (279 mg, 4.24 mmol) and copper (II) acetate (51 mg, 0.28 mmol) are suspended in MeOH (10 mL). The reaction mixture is stirred at 60° C. for 3 h under an air atmosphere. The mixture is filtered, the solvent removed under reduced pressure and the residue purified by FC (silica gel, Et₂O) to give 4-azido-2-chlorophenol. LC-MS (basic): t_{R}=0.40; [M+H]⁺=not detected.

### Step 2

Prepared following the procedure described for Example 1 using Intermediate A, rac-1-(6-cyclopropylimidazo[1,5-a]pyridin-5-yl)prop-2-yn-1-ol and 4-azido-2-chlorophenol. Purification by prepHPLC (basic conditions) to give rac-2-chloro-4-{4-[(6-cyclopropyl-imidazo[1,5-a]pyridin-5-yl)-hydroxy-methyl]-[1,2,3]triazol-1-yl}-phenol. LC-MS (QC): t_{R}=0.601; [M+H]⁺=382.2.

### Examples 7a and 7b: 2-Chloro-4-{4-[(R)-(6-cyclopropyl-imidazo[1,5-a]pyridin-5-yl)-hydroxy-methyl]-[1,2,3]triazol-1-yl}-phenol and 2-Chloro-4-{4-[(S)-(6-cyclopropyl-imidazo[1,5-a]pyridin-5-yl)-hydroxy-methyl]-[1,2,3]triazol-1-yl}-phenol

Separation of rac-2-chloro-4-{4-[(6-cyclopropyl-imidazo[1,5-a]pyridin-5-yl)-hydroxy-methyl]-[1,2,3]triazol-1-yl}-phenol on chiral stationary phase. Method: Column: ChiralPak IA 30×250 mm, 5 μM; Detector Settings: UV-Vis-1; 210 nM; Eluent: 55% CO₂ and 45% (CH₂Cl₂/EtOH 1:1); Flow: 160.00 mL/min; BPR: 100 bar; Temperature: 40° C. Injection volume: 1000 μl.

10.5 mg of the racemate are separated by the method described above to give:

2 mg of the R-enantiomer Example 7a and 4 mg of the S-enantiomer Example 7b.

Example 7a: LC-MS (QC): t_{R}=0.602; [M+H]⁺=382.2.

Example 7b: LC-MS (QC): t_{R}=0.601; [M+H]⁺=382.2.

### Example 8: rac-[1-(3-Chloro-4-methoxy-phenyl)-1H-[1,2,3]triazol-4-yl]-(6-cyclopropyl-imidazo[1,5-a]pyridin-5-yl)-methanol

### Step 1: Preparation of 4-azido-2-chloro-1-methoxybenzene

To a solution of 3-chloro-4-methoxyaniline (500 mg; 3.11 mmol) in 1M aq. HCl (40 mL) is added at 0° C. a solution of sodium nitrite (217 mg; 3.11 mmol) in water (8 mL). The reaction mixture is stirred for 20 minutes, and sodium azide (245 mg; 3.73 mmol) is added. The reaction mixture is stirred at RT for 3 h. The mixture is diluted with EtOAc, the layers separated and the org. layer washed with brine, dried (MgSO₄), filtered and concentrated under reduced pressure to give 4-azido-2-chloro-1-methoxybenzene as a brown oil. ¹H NMR (400 MHz, DMSO) δ: 7.23 (d, J=2.7 Hz, 1H), 7.18 (m, 1H), 7.10 (dd, J1=2.7 Hz, J2=8.8 Hz, 1H), 3.85 (s, 3H).

### Step 2

Prepared following the procedure described for Example 1, and using Intermediate A, rac-1-(6-cyclopropylimidazo[1,5-a]pyridin-5-yl)prop-2-yn-1-ol and 4-azido-2-chloro-1-methoxybenzene. Purification by prepHPLC (basic conditions) to give rac-[1-(3-chloro-4-methoxy-phenyl)-1H-[1,2,3]triazol-4-yl]-(6-cyclopropyl-imidazo[1,5-a]pyridin-5-yl)-methanol. LC-MS (QC): t_{R}=0.705; [M+H]⁺=396.2. ¹H NMR (500 MHz, DMSO) δ: 9.02 (s, 1H), 8.35-8.72 (m, 1H), 8.04-8.08 (m, 1H), 7.90 (dd, J1=8.9 Hz, J2=2.5 Hz, 1H), 7.48 (d, J=9.3 Hz, 1H), 7.35 (d, J=9.0 Hz, 1H), 7.49-7.28 (br s, 1H), 7.01 (d, J=3.9 Hz, 1H), 6.59-6.71 (m, 2H), 3.93 (s, 3H), 2.20 (m, 1H), 0.94-1.10 (m, 2H), 0.73-0.94 (m, 2H).

### Example 8a: (R)-[1-(3-Chloro-4-methoxy-phenyl)-1H-[1,2,3]triazol-4-yl]-(6-cyclopropyl-imidazo[1,5-a]pyridin-5-yl)-methanol

Separation of rac-[1-(3-chloro-4-methoxy-phenyl)-1H-[1,2,3]triazol-4-yl]-(6-cyclopropyl-imidazo[1,5-a]pyridin-5-yl)-methanol on chiral stationary phase. Method: Column: ChiralPak AS-H 30×250 mm, 5 μM; Detector Settings: UV 259 nm; Eluent: 30% CO₂ and 70% MeCN/EtOH/DEA 50:50:0.1; Flow: 160.00 mL/min; BPR: 120 bar; Temperature: 40° C. Injection volume: 3000 μl.

12.4 mg of the racemate are separated by the method described above to give:

1.9 mg of the R-enantiomer Example 8a and 5.2 mg of the S-enantiomer.

Example 8a: LC-MS (QC): t_{R}=0.705; [M+H]⁺=396.2.

### Example 9: rac-(6-Cyclopropyl-imidazo[1,5-a]pyridin-5-yl)-[1-(6-ethoxy-pyridin-3-yl)-1H-[1,2,3]triazol-4-yl]-methanol

### Step 1: Preparation of 5-azido-2-ethoxypyridine

Prepared according to the procedure described for Example 7, step 1 using 2-ethoxypyridine-5-boronic acid.

### Step 2

Prepared following the procedure described for Example 1 using Intermediate A, rac-1-(6-cyclopropylimidazo[1,5-a]pyridin-5-yl)prop-2-yn-1-ol and 5-azido-2-ethoxypyridine. Purification by prepHPLC (basic conditions) to give rac-(6-cyclopropyl-imidazo[1,5-a]pyridin-5-yl)-[1-(6-ethoxy-pyridin-3-yl)-1H-[1,2,3]triazol-4-yl]-methanol. LC-MS (QC): t_{R}=0.652; [M+H]⁺=377.2. ¹H NMR (500 MHz, DMSO) δ: 9.00 (s, 1H), 8.66 (d, J=2.6 Hz, 1H), 8.39-8.61 (m, 1H), 8.22 (dd, J1=8.9 Hz, J2=2.8 Hz, 1H), 7.49 (d, J=9.4 Hz, 1H), 7.22-7.44 (m, 1H), 6.96-7.06 (m, 2H), 6.70 (d, J=4.0 Hz, 1H), 6.66 (d, J=9.4 Hz, 1H), 4.37 (q, J=7.0 Hz, 2H), 2.16-2.25 (m, 1H), 1.35 (t, J=7.0 Hz, 3H), 0.95-1.07 (m, 2H), 0.76-0.91 (m, 2H).

### Example 10: rac-2-Chloro-4-{4-[(6-cyclopropyl-imidazo[1,5-a]pyrazin-5-yl)-hydroxy-methyl]-[1,2,3]triazol-1-yl}-phenol

Prepared following the procedure described for Example 7 using Intermediate B, rac-1-(6-cyclopropylimidazo[1,5-a]pyrazin-5-yl)prop-2-yn-1-ol and 4-azido-2-chlorophenol. Purification by prepHPLC (basic conditions) to give rac-2-chloro-4-{4-[(6-cyclopropyl-imidazo[1,5-a]pyrazin-5-yl)-hydroxy-methyl]-[1,2,3]triazol-1-yl}-phenol. LC-MS (QC): t_{R}=0.703; [M+H]⁺=383.1. ¹H NMR (500 MHz, DMSO) δ: 8.94-8.96 (m, 2H), 8.63 (s, 1H), 7.95 (d, J=2.7 Hz, 1H), 7.77 (s, 1H), 7.70 (dd, J1=8.8 Hz, J2=2.7 Hz, 1H), 7.12 (d, J=8.8 Hz, 1H), 6.85 (d, J=3.3 Hz, 1H), 6.77 (d, J=3.9 Hz, 1H), 2.43-2.48 (m, 1H), 1.05-1.10 (m, 1H), 0.94-1.00 (m, 3H).

### Example 10a: 2-Chloro-4-{4-[(R)-(6-cyclopropyl-imidazo[1,5-a]pyrazin-5-yl)-hydroxy-methyl]-[1,2,3]triazol-1-yl}-phenol

Prepared following the procedure described for Example 7 using Intermediate Ba, (S)-1-(6-cyclopropylimidazo[1,5-a]pyrazin-5-yl)prop-2-yn-1-ol and 4-azido-2-chlorophenol. Purification by prepHPLC (basic conditions) to give 2-chloro-4-{4-[(R)-(6-cyclopropyl-imidazo[1,5-a]pyrazin-5-yl)-hydroxy-methyl]-[1,2,3]triazol-1-yl}-phenol. LC-MS (QC): t_{R}=0.702; [M+H]⁺=383.1.

### Example 11: rac-[1-(3-Chloro-4-methoxy-phenyl)-1H-[1,2,3]triazol-4-yl]-(6-cyclopropyl-imidazo[1,5-a]pyrazin-5-yl)-methanol

Prepared following the procedure described for Example 8 using Intermediate B, rac-1-(6-cyclopropylimidazo[1,5-a]pyrazin-5-yl)prop-2-yn-1-ol and 4-azido-2-chloro-1-methoxybenzene. Purification by prepHPLC (basic conditions) to give rac-[1-(3-chloro-4-methoxy-phenyl)-1H-[1,2,3]triazol-4-yl]-(6-cyclopropyl-imidazo[1,5-a]pyrazin-5-yl)-methanol. LC-MS (QC): t_{R}=0.843; [M+H]⁺=397.2. ¹H NMR (500 MHz, DMSO) δ: 9.02 (d, 1H), 8.96 (s, 1H), 8.63 (s, 1H), 8.06 (d, J=2.7 Hz, 1H), 7.89 (dd, J1=2.7 Hz, J2=8.9 Hz, 1H), 7.77 (d, 1H), 7.35 (d, J=9.1 Hz, 1H), 6.86 (d, J=2.0 Hz, 1H), 6.78 (d, J=2.7 Hz, 1H), 3.94 (s, 3H), 2.44-2.48 (m, 1H), 1.04-1.14 (m, 1H), 0.91-1.05 (m, 3H).

### Example 11a: (R)-[1-(3-Chloro-4-methoxy-phenyl)-1H-[1,2,3]triazol-4-yl]-(6-cyclopropyl-imidazo[1,5-a]pyrazin-5-yl)-methanol

Separation of rac-[1-(3-chloro-4-methoxy-phenyl)-1H-[1,2,3]triazol-4-yl]-(6-cyclopropyl-imidazo[1,5-a]pyrazin-5-yl)-methanol on chiral stationary phase. Method: Column: ChiralCel OD-H 30×250 mm, 5 μM; Detector Wavelength: UV 223 nM; Eluent: 35% CO₂ and 65% (MeCN/EtOH 1:1); Flow: 160.00 mL/min; BPR: 100 bar; Temperature: 40° C. Injection volume: 4000 μl.

21.1 mg of the racemate are separated by the method described above to give:

9 mg of the R-enantiomer Example 11a and 8.8 mg of the S-enantiomer.

Example 11a: LC-MS (QC): t_{R}=0.843; [M+H]⁺=397.2.

### Example 12: rac-(4-{4-[(6-Cyclopropyl-imidazo[1,5-a]pyrazin-5-yl)-hydroxy-methyl]-[1,2,3]triazol-1-yl}-phenyl)-carbamic acid methyl ester

Prepared following the procedure described for Example 6 using Intermediate B, rac-1-(6-cyclopropylimidazo[1,5-a]pyrazin-5-yl)prop-2-yn-1-ol and methyl (4-azidophenyl)carbamate. Purification by prepHPLC (basic conditions) to give rac-(4-{4-[(6-cyclopropyl-imidazo[1,5-a]pyrazin-5-yl)-hydroxy-methyl]-[1,2,3]triazol-1-yl}-phenyl)-carbamic acid methyl ester. LC-MS (QC): t_{R}=0.685; [M+H]⁺=406.2. ¹H NMR (500 MHz, DMSO) δ: 9.94 (s, 1H), 8.94-8.96 (m, 2H), 8.63 (s, 1H), 7.82 (d, J=9.1 Hz, 2H), 7.77 (s, 1H), 7.64 (d, J=9.0 Hz, 2H), 6.86 (d, J=3.8 Hz, 1H), 6.78 (d, J=4.0 Hz, 1H), 3.70 (s, 3H), 2.44-2.48 (m, 1H), 1.05-1.11 (m, 1H), 0.92-1.02 (m, 3H).

### Example 13: rac-(6-Cyclopropyl-imidazo[1,5-a]pyrazin-5-yl)-[1-(6-ethoxy-pyridin-3-yl)-1H-[1,2,3]triazol-4-yl]-methanol

Prepared following the procedure described for Example 9 using Intermediate B, rac-1-(6-cyclopropylimidazo[1,5-a]pyrazin-5-yl)prop-2-yn-1-ol and 5-azido-2-ethoxypyridine. Purification by prepHPLC (basic conditions) to give rac-(6-cyclopropyl-imidazo[1,5-a]pyrazin-5-yl)-[1-(6-ethoxy-pyridin-3-yl)-1H-[1,2,3]triazol-4-yl]-methanol. LC-MS (QC): t_{R}=0.782; [M+H]⁺=378.2. ¹H NMR (500 MHz, DMSO) δ: 9.00 (d, 1H), 8.96 (s, 1H), 8.69 (dd, J=2.8 Hz, 1H), 8.61 (s, 1H), 8.21 (dd, J1=8.9 Hz, J2=2.8 Hz, 1H), 7.77 (d, 1H), 7.02 (dd, J=8.9 Hz, 1H), 6.87 (d, J=3.8 Hz, 1H), 6.80 (d, J=4.0 Hz, 1H), 4.37 (q, J=7.0 Hz, 2H), 2.46 (m, 1H), 1.35 (t, J=7.0 Hz, 3H), 1.05-1.10 (m, 1H), 0.97-1.02 (m, 1H), 0.94-0.97 (m, 2H).

### Example 14: rac-4-{4-[(6-Cyclopropyl-imidazo[1,5-a]pyridin-5-yl)-hydroxy-methyl]-[1,2,3]triazol-1-yl}-phenol

### Step 1: Preparation of 4-azidophenol

Prepared according to the procedure described for Example 7, step 1 using (4-hydroxyphenyl)boronic acid.

### Step 2

Prepared following the procedure described for Example 1 using Intermediate A, rac-1-(6-cyclopropylimidazo[1,5-a]pyridin-5-yl)prop-2-yn-1-ol and 4-azidophenol. Purification by prepHPLC (basic conditions) to give rac-4-{4-[(6-cyclopropyl-imidazo[1,5-a]pyridin-5-yl)-hydroxy-methyl]-[1,2,3]triazol-1-yl}-phenol. LC-MS (QC): t_{R}=0.518; [M+H]⁺=348.2.

### Example 15: rac-4-{4-[(6-Cyclopropyl-imidazo[1,5-a]pyrazin-5-yl)-hydroxy-methyl]-[1,2,3]triazol-1-yl}-phenol

Prepared following the procedure described for Example 14 using Intermediate B, rac-1-(6-cyclopropylimidazo[1,5-a]pyrazin-5-yl)prop-2-yn-1-ol and 4-azidophenol. Purification by prepHPLC (basic conditions) to give rac-4-{4-[(6-cyclopropyl-imidazo[1,5-a]pyrazin-5-yl)-hydroxy-methyl]-[1,2,3]triazol-1-yl}-phenol. LC-MS (QC): t_{R}=0.597; [M+H]⁺=349.1.

### Example 16: rac-(6-Cyclopropyl-imidazo[1,5-a]pyrazin-5-yl)-{1-[4-(3-fluoro-oxetan-3-ylmethoxy)-phenyl]-1H-[1,2,3]triazol-4-yl}-methanol

A mixture of Example 15, rac-4-{4-[(6-cyclopropyl-imidazo[1,5-a]pyrazin-5-yl)-hydroxy-methyl]-[1,2,3]triazol-1-yl}-phenol (34.8 mg, 0.1 mmol), 3-(bromomethyl)-3-fluorooxetane (69 mg, 0.4 mmol), sodium iodide (0.757 mg, 0.005 mmol) and K₂CO₃ (55.3 mg, 0.4 mmol) in DMF (2 mL) is stirred at 50° C. for 16 h. The mixture is then filtered and purified by preparative HPLC (basic conditions) to give 31.7 mg of rac-(6-cyclopropyl-imidazo[1,5-a]pyrazin-5-yl)-{1-[4-(3-fluoro-oxetan-3-ylmethoxy)-phenyl]-1H-[1,2,3]triazol-4-yl}-methanol as a white solid. LC-MS (QC): t_{R}=0.760; [M+H]⁺=437.2. ¹H NMR (500 MHz, DMSO) δ: 8.96-8.97 (m, 2H), 8.65 (s, 1H), 7.86 (d, J=9.1 Hz, 2H), 7.77 (d, 1H), 7.20 (d, J=9.1 Hz, 2H), 6.86 (d, J=3.8 Hz, 1H), 6.77 (d, J=4.0 Hz, 1H), 4.68-4.78 (m, 4H), 4.52 (d, J=22.1 Hz, 2H), 2.43-2.47 (m, 1H), 1.05-1.13 (m, 1H), 0.91-1.04 (m, 3H).

### Example 17: rac-(6-Cyclopropyl-imidazo[1,5-a]pyrazin-5-yl)-[1-(4-methoxy-phenyl)-1H-[1,2,3]triazol-4-yl]-methanol

Prepared following the procedure described for Example 1 using Intermediate B, rac-1-(6-cyclopropylimidazo[1,5-a]pyrazin-5-yl)prop-2-yn-1-ol and 1-azido-4-methoxybenzene. Purification by preparative HPLC (basic conditions) gives rac-(6-cyclopropyl-imidazo[1,5-a]pyrazin-5-yl)-[1-(4-methoxy-phenyl)-1H-[1,2,3]triazol-4-yl]-methanol. LC-MS (QC): t_{R}=0.747 min, [M+H]⁺=363.2. ¹H NMR (500 MHz, DMSO) δ: 8.94-8.96 (m, 2H), 8.64 (s, 1H), 7.82 (d, J=9.1 Hz, 2H), 7.77 (d, 1H), 7.13 (d, J=9.1 Hz, 2H), 6.86 (s, 1H), 6.77 (s, 1H), 3.83 (s, 3H), 2.46 (m, 1H), 1.06-1.09 (m, 1H), 0.93-1.00 (m, 3H).

### Example 18: rac-1-(4-{4-[(6-Cyclopropyl-imidazo[1,5-a]pyrazin-5-yl)-hydroxy-methyl]-[1,2,3]triazol-1-yl}-phenoxy)-2-methyl-propan-2-ol

Prepared following the procedure described for Example 16 using Example 15, rac-4-{4-[(6-cyclopropyl-imidazo[1,5-a]pyrazin-5-yl)-hydroxy-methyl]-[1,2,3]triazol-1-yl}-phenol and 1-bromo-2-methylpropan-2-ol. Purification by preparative HPLC (basic conditions) gives rac-1-(4-{4-[(6-cyclopropyl-imidazo[1,5-a]pyrazin-5-yl)-hydroxy-methyl]-[1,2,3]triazol-1-yl}-phenoxy)-2-methyl-propan-2-ol. LC-MS (QC): t_{R}=0.725 min, [M+H]⁺=421.3. ¹H NMR (500 MHz, DMSO) δ: 8.94-8.96 (m, 2H), 8.65 (s, 1H), 7.81 (d, J=9.1 Hz, 2H), 7.77 (d, 1H), 7.13 (m, 2H), 6.86 (d, J=3.9 Hz, 1H), 6.76 (d, J=4.0 Hz, 1H), 4.67 (s, 1H), 3.79 (s, 2H), 2.44-2.48 (m, 1H), 1.22 (s, 6H), 1.06-1.09 (m, 1H), 0.94-1.00 (m, 3H).

### Example 19: rac-(6-Cyclopropyl-imidazo[1,5-a]pyridin-5-yl)-{1-[4-(3-fluoro-oxetan-3-ylmethoxy)-phenyl]-1H-[1,2,3]triazol-4-yl}-methanol

Prepared following the procedure described for Example 16 using Example 14, rac-4-{4-[(6-cyclopropyl-imidazo[1,5-a]pyridin-5-yl)-hydroxy-methyl]-[1,2,3]triazol-1-yl}-phenol and 3-(bromomethyl)-3-fluorooxetane. Purification by preparative HPLC (basic conditions) gives rac-(6-cyclopropyl-imidazo[1,5-a]pyridin-5-yl)-{1-[4-(3-fluoro-oxetan-3-ylmethoxy)-phenyl]-1H-[1,2,3]triazol-4-yl}-methanol. LC-MS (QC): t_{R}=0.645 min, [M+H]⁺=436.3. ¹H NMR (500 MHz, DMSO) δ: 8.91-9.06 (m, 1H), 8.39-8.57 (m, 1H), 7.80-7.91 (m, 2H), 7.44-7.53 (m, 1H), 7.29-7.37 (m, 1H), 7.13-7.26 (m, 2H), 6.94-7.05 (m, 1H), 6.63-6.69 (m, 2H), 4.61-4.85 (m, 4H), 4.44-4.60 (m, 2H), 2.16-2.32 (m, 1H), 0.94-1.17 (m, 2H), 0.72-0.93 (m, 2H).

### Example 20: rac-(6-Chloro-imidazo[1,5-a]pyridin-5-yl)-[1-(4-methoxy-phenyl)-5-methyl-1H-[1,2,3]triazol-4-yl]-methanol

### Step 1: Preparation of ethyl 1-(4-methoxyphenyl)-5-methyl-1H-1,2,3-triazole-4-carboxylate

A suspension of ethyl acetoacetate (0.21 ml, 1.67 mmol), 1-azido-4-methoxybenzene (193 mg, 1.29 mmol) and K₂CO₃ (714 mg, 5.17 mmol) in DMSO (0.8 mL) is stirred at 50° C. for 1h30. The mixture is cooled down to RT, diluted with water and EtOAc, and acidified with aq. 1N HCl. The layers are separated and the aq. layer extracted with EtOAc (2×). The combined org. extracts are dried (MgSO₄), filtered and concentrated under reduced pressure. The crude solid is triturated in Et₂O/Petroleum ether and filtered to give 165 mg of ethyl 1-(4-methoxyphenyl)-5-methyl-1H-1,2,3-triazole-4-carboxylate as a beige solid. LC-MS (acidic): t_{R}=0.86, [M+H]⁺=262.19.

### Step 2: Preparation of (1-(4-methoxyphenyl)-5-methyl-1H-1,2,3-triazol-4-yl)methanol

To a solution of ethyl 1-(4-methoxyphenyl)-5-methyl-1H-1,2,3-triazole-4-carboxylate (165 mg, 0.63 mmol) in EtOH (8.7 mL) is added NaBH₄ (218 mg, 5.76 mmol) at RT. The reaction mixture is stirred at RT for 48 h. The reaction mixture is concentrated under reduced pressure and the residue partitioned between DCM and water. The layers are separated and the aq. layer extracted with DCM (2×). The combined org. extracts are dried (MgSO₄), filtered and concentrated under reduced pressure. The crude solid is triturated with Et₂O/Petroleum ether and filtered to give 126 mg of (1-(4-methoxyphenyl)-5-methyl-1H-1,2,3-triazol-4-yl)methanol as a beige solid. LC-MS (acidic): t_{R}=0.60, [M+H]⁺=220.27.

### Step 3: Preparation of 1-(4-methoxyphenyl)-5-methyl-1H-1,2,3-triazole-4-carbaldehyde

To a solution of (1-(4-methoxyphenyl)-5-methyl-1H-1,2,3-triazol-4-yl)methanol (126 mg, 0.58 mmol) in CH₂Cl₂ (7 mL) is added Dess-Martin periodinane (271 mg, 0.64 mmol). The reaction mixture is stirred at RT for until completion of the reaction. Sat. aq. NaHCO₃ and sat. aq. Na₂S₂O₃ are added and the mixture is stirred at RT for 10 min. The layers are separated and the aq. layer extracted with CH₂Cl₂. The combined org. extracts are washed with brine, dried (MgSO₄), filtered and concentrated under reduced pressure to give 53 mg of 1-(4-methoxyphenyl)-5-methyl-1H-1,2,3-triazole-4-carbaldehyde as a white solid after preparative HPLC (basic conditions). LC-MS (basic): t_{R}=0.76, [M+H]⁺=218.16.

### Step 4: Preparation of rac-(6-chloro-imidazo[1,5-a]pyridin-5-yl)-[1-(4-methoxy-phenyl)-5-methyl-1H-[1,2,3]triazol-4-yl]-methanol (Example 20)

To a solution of 6-chloroimidazo[1,5-a]pyridine (30 mg, 0.20 mmol) in dry THF (2.3 mL) is added n-BuLi (2.5 M in hexanes, 0.16 mL, 0.40 mmol) at −78° C. The resulting brown solution is stirred at −78° C. for 45 min. A solution of 1-(4-methoxyphenyl)-5-methyl-1H-1,2,3-triazole-4-carbaldehyde (45 mg, 0.21 mmol) in dry THF (1 mL) is the added and the reaction mixture stirred at −78° C. for 1 h and slowly warmed up to 0° C. Sat. aq. NH₄Cl solution is added and the mixture extracted with EtOAc (3×). The combined org. extracts are dried (MgSO₄), filtered and concentrated under reduced pressure. Purification by preparative HPLC (acidic conditions) gives rac-(6-chloro-imidazo[1,5-a]pyridin-5-yl)-[1-(4-methoxy-phenyl)-5-methyl-1H-[1,2,3]triazol-4-yl]-methanol. LC-MS (QC): t_{R}=0.695 min, [M+H]⁺=370.1. The title compound (38 mg) is separated on chiral stationary phase using the following Method: Column: ChiralPak IH 30×250 mm, 5 μM; Detector Wavelength: 222 nm; Eluent: 55% CO₂ and 45% (MeCN/EtOH/DEA 50:50:0.1); Flow: 160.00 mL/min; BPR: 100 bar; Temperature: 40° C.; Injection volume: 1000 pit to give 13 mg of (R)-(6-Chloro-imidazo[1,5-a]pyridin-5-yl)-[1-(4-methoxy-phenyl)-5-methyl-1H-[1,2,3]triazol-4-yl]-methanol (Example 20a) and 14 mg of the S-enantiomer.

Example 20a: LC-MS (QC): t_{R}=0.681 min, [M+H]⁺=370.3.

### Example 21: (R)-(6-Cyclopropyl-imidazo[1,5-a]pyrazin-5-yl)-[1-(3-fluoro-4-methoxy-phenyl)-1H-[1,2,3]triazol-4-yl]-methanol

### Step 1: Preparation of 4-azido-2-fluoro-1-methoxybenzene

Prepared according to the procedure described for Example 7, step 1 using (3-fluoro-4-methoxyphenyl)boronic acid.

### Step 2

Prepared following the procedure described for Example 1 using Intermediate Ba, (S)-1-(6-cyclopropylimidazo[1,5-a]pyrazin-5-yl)prop-2-yn-1-ol and 4-azido-2-fluoro-1-methoxybenzene. Purification by prepHPLC (acidic conditions) to give (R)-(6-cyclopropyl-imidazo[1,5-a]pyrazin-5-yl)-[1-(3-fluoro-4-methoxy-phenyl)-1H-[1,2,3]triazol-4-yl]-methanol. LC-MS (QC): t_{R}=0.780; [M+H]⁺=381.2. ¹H NMR (500 MHz, DMSO) δ: 9.00 (d, J=0.6 Hz, 1H), 8.96 (s, 1H), 8.62 (s, 1H), 7.89 (dd, J1=12.1 Hz, J2=2.6 Hz, 1H), 7.77 (s, 1H), 7.74 (ddd, J1=8.9 Hz, J2=2.6 Hz, J3=1.5 Hz, 1H), 7.38 (t, J=9.1 Hz, 1H), 6.86 (d, J=3.7 Hz, 1H), 6.79 (d, J=4.0 Hz, 1H), 3.92 (s, 3H), 2.47 (m, 1H), 1.05-1.08 (m, 1H), 0.93-1.00 (m, 3H).

### Example 22: (R)-(6-Cyclopropyl-imidazo[1,5-a]pyrazin-5-yl)-[1-(2,5-difluoro-4-methoxy-phenyl)-1H-[1,2,3]triazol-4-yl]-methanol

### Step 1: Preparation of 1-azido-2,5-difluoro-4-methoxybenzene

Prepared according to the procedure described for Example 7, step 1 using (2,5-difluoro-4-methoxyphenyl)boronic acid.

### Step 2

Prepared following the procedure described for Example 1 using Intermediate Ba, (S)-1-(6-cyclopropylimidazo[1,5-a]pyrazin-5-yl)prop-2-yn-1-ol and 1-azido-2,5-difluoro-4-methoxybenzene. Purification by prepHPLC (acidic conditions) to give (R)-(6-cyclopropyl-imidazo[1,5-a]pyrazin-5-yl)-[1-(2,5-difluoro-4-methoxy-phenyl)-1H-[1,2,3]triazol-4-yl]-methanol. LC-MS (QC): t_{R}=0.799; [M+H]⁺=399.2. ¹H NMR (500 MHz, DMSO) δ: 8.96 (s, 1H), 8.76 (d, J=1.0 Hz, 1H), 8.62 (s, 1H), 7.83 (dd, J1=7.1 Hz, J2=11.2 Hz, 1H), 7.78 (s, 1H), 7.52 (dd, J1=7.6 Hz, J2=12.2 Hz, 1H), 6.88 (d, J=3.6 Hz, 1H), 6.80 (d, J=4.0 Hz, 1H), 3.94 (s, 3H), 2.46-2.48 (m, 1H), 1.05-1.08 (m, 1H), 0.93-1.01 (m, 3H).

### Example 23: (R)-[1-(3-Bromo-4-methoxy-phenyl)-1H-[1,2,3]triazol-4-yl]-(6-cyclopropyl-imidazo[1,5-a]pyrazin-5-yl)-methanol

### Step 1: Preparation of 4-azido-2-bromo-1-methoxybenzene

Prepared according to the procedure described for Example 8, step 1 using 3-bromo-4-methoxyaniline.

### Step 2

Prepared following the procedure described for Example 1 using Intermediate Ba, (S)-1-(6-cyclopropylimidazo[1,5-a]pyrazin-5-yl)prop-2-yn-1-ol and 4-azido-2-bromo-1-methoxybenzene. Purification by prepHPLC (acidic conditions) to give (R)-[1-(3-bromo-4-methoxy-phenyl)-1H-[1,2,3]triazol-4-yl]-(6-cyclopropyl-imidazo[1,5-a]pyrazin-5-yl)-methanol. LC-MS (QC): t_{R}=0.863; [M+H]⁺=441.1. ¹H NMR (500 MHz, DMSO) δ: 9.02 (s, 1H), 8.96 (s, 1H), 8.65 (s, 1H), 8.19 (d, J=2.7 Hz, 1H), 7.93 (dd, J1=8.9 Hz, J2=2.7 Hz, 1H), 7.78 (br s, 1H), 7.31 (d, J=9.1 Hz, 1H), 6.86 (s, 1H), 6.78 (br s, 1H), 3.93 (s, 3H), 2.44-2.48 (m, 1H), 1.06-1.09 (m, 1H), 0.94-1.00 (m, 3H).

### Example 24: (R)-(6-Cyclopropyl-imidazo[1,5-a]pyrazin-5-yl)-[1-(4-methoxymethyl-phenyl)-1H-[1,2,3]triazol-4-yl]-methanol

### Step 1: Preparation of 1-azido-4-(methoxymethyl)benzene

Prepared according to the procedure described for Example 8, step 1 using 4-(methoxymethyl)aniline.

### Step 2

Prepared following the procedure described for Example 1 using Intermediate Ba, (S)-1-(6-cyclopropylimidazo[1,5-a]pyrazin-5-yl)prop-2-yn-1-ol and 1-azido-4-(methoxymethyl)benzene. Purification by prepHPLC (acidic conditions) to give (R)-(6-cyclopropyl-imidazo[1,5-a]pyrazin-5-yl)-[1-(4-methoxymethyl-phenyl)-1H-[1,2,3]triazol-4-yl]-methanol. LC-MS (QC): t_{R}=0.741; [M+H]⁺=377.2. ¹H NMR (500 MHz, DMSO) δ: 9.05 (d, 1H), 8.96 (s, 1H), 8.65 (s, 1H), 7.91 (d, J=8.6 Hz, 2H), 7.78 (s, 1H), 7.53 (d, J=8.6 Hz, 2H), 6.88 (d, J=3.8 Hz, 1H), 6.79 (d, J=4.0 Hz, 1H), 4.49 (s, 2H), 3.33 (s, 3H), 2.47 (m, 1H), 1.05-1.10 (m, 1H), 0.94-1.01 (m, 3H).

### Example 25: (R)-[1-(5-Chloro-2-fluoro-4-methoxy-phenyl)-1H-[1,2,3]triazol-4-yl]-(6-cyclopropyl-imidazo[1,5-a]pyrazin-5-yl)-methanol

### Step 1: Preparation of 1-azido-5-chloro-2-fluoro-4-methoxybenzene

Prepared according to the procedure described for Example 8, step 1 using 5-chloro-2-fluoro-4-methoxyaniline.

### Step 2

Prepared following the procedure described for Example 1 using Intermediate Ba, (S)-1-(6-cyclopropylimidazo[1,5-a]pyrazin-5-yl)prop-2-yn-1-ol and 1-azido-5-chloro-2-fluoro-4-methoxybenzene. Purification by prepHPLC (acidic conditions) to give (R)-[1-(5-chloro-2-fluoro-4-methoxy-phenyl)-1H-[1,2,3]triazol-4-yl]-(6-cyclopropyl-imidazo[1,5-a]pyrazin-5-yl)-methanol. LC-MS (QC): t_{R}=0.861; [M+H]⁺=415.1. ¹H NMR (500 MHz, DMSO) δ: 8.96 (s, 1H), 8.76 (d, 1H), 8.63 (s, 1H), 7.97 (d, J=7.8 Hz, 1H), 7.76-7.79 (m, 1H), 7.49 (d, J=12.4 Hz, 1H), 6.88 (s, 1H), 6.76-6.83 (m, 1H), 3.97 (s, 3H), 2.46-2.48 (m, 1H), 1.05-1.08 (m, 1H), 0.93-1.00 (m, 3H).

### Example 26: (R)-[1-(3-Chloro-5-fluoro-4-methoxy-phenyl)-1H-[1,2,3]triazol-4-yl]-(6-cyclopropyl-imidazo[1,5-a]pyrazin-5-yl)-methanol

### Step 1: Preparation of 5-azido-1-chloro-3-fluoro-2-methoxybenzene

Prepared according to the procedure described for Example 8, step 1 using 3-chloro-5-fluoro-4-methoxyaniline.

### Step 2

Prepared following the procedure described for Example 1 using Intermediate Ba, (S)-1-(6-cyclopropylimidazo[1,5-a]pyrazin-5-yl)prop-2-yn-1-ol and 5-azido-1-chloro-3-fluoro-2-methoxybenzene. Purification by prepHPLC (acidic conditions) to give (R)-[1-(3-chloro-5-fluoro-4-methoxy-phenyl)-1H-[1,2,3]triazol-4-yl]-(6-cyclopropyl-imidazo[1,5-a]pyrazin-5-yl)-methanol. LC-MS (QC): t_{R}=0.920; [M+H]⁺=415.2. ¹H NMR (500 MHz, DMSO) δ: 9.08 (d, J=0.5 Hz, 1H), 8.96 (s, 1H), 8.61 (s, 1H), 7.99-8.02 (m, 2H), 7.74-7.82 (m, 1H), 6.87 (m, 1H), 6.83 (m, 1H), 3.96 (d, J=1.4 Hz, 3H), 2.47 (m, 1H), 1.06-1.09 (m, 1H), 0.92-1.01 (m, 3H).

### Example 27: 4-{4-[(R)-(6-Cyclopropyl-imidazo[1,5-a]pyrazin-5-yl)-hydroxy-methyl]-[1,2,3]triazol-1-yl}-2-fluoro-phenol

### Step 1: Preparation of 4-azido-2-fluorophenol

Prepared according to the procedure described for Example 7, step 1 using 2-fluoro-4-(4,4,5,5-tetramethyl-1,3,2-dioxaborolan-2-yl)phenol.

### Step 2

Prepared following the procedure described for Example 1 using Intermediate Ba, (S)-1-(6-cyclopropylimidazo[1,5-a]pyrazin-5-yl)prop-2-yn-1-ol and 4-azido-2-fluorophenol. Purification by prepHPLC (acidic conditions) to give 4-{4-[(R)-(6-cyclopropyl-imidazo[1,5-a]pyrazin-5-yl)-hydroxy-methyl]-[1,2,3]triazol-1-yl}-2-fluoro-phenol. LC-MS (QC): t_{R}=0.642; [M+H]⁺=367.2.

### Example 28: (R)-(6-Cyclopropyl-imidazo[1,5-a]pyrazin-5-yl)-{1-[3-fluoro-4-(3-fluoro-oxetan-3-ylmethoxy)-phenyl]-1H-[1,2,3]triazol-4-yl}-methanol

Prepared following the procedure described for Example 16 using Example 27, 4-{4-[(R)-(6-cyclopropyl-imidazo[1,5-a]pyrazin-5-yl)-hydroxy-methyl]-[1,2,3]triazol-1-yl}-2-fluoro-phenol and 3-(bromomethyl)-3-fluorooxetane. Purification by preparative HPLC (basic conditions) gives (R)-(6-cyclopropyl-imidazo[1,5-a]pyrazin-5-yl)-{1-[3-fluoro-4-(3-fluoro-oxetan-3-ylmethoxy)-phenyl]-1H-[1,2,3]triazol-4-yl}-methanol. LC-MS (QC): t_{R}=0.795 min, [M+H]⁺=455.2. ¹H NMR (500 MHz, DMSO) δ: 9.01 (d, J=0.6 Hz, 1H), 8.96 (s, 1H), 8.62 (s, 1H), 7.93 (dd, J1=11.9 Hz, J2=2.6 Hz, 1H), 7.75-7.78 (m, 2H), 7.45 (t, J=9.0 Hz, 1H), 6.87 (d, J=3.8 Hz, 1H), 6.80 (d, J=4.0 Hz, 1H), 4.69-4.78 (m, 4H), 4.61 (d, J=22.2 Hz, 2H), 2.46 (m, 1H), 1.06-1.09 (m, 1H), 0.94-1.01 (m, 3H).

### Example 29: 1-(4-{4-[(R)-(6-Cyclopropyl-imidazo[1,5-a]pyrazin-5-yl)-hydroxy-methyl]-[1,2,3]triazol-1-yl}-2-fluoro-phenoxy)-2-methyl-propan-2-ol

Prepared following the procedure described for Example 16 using Example 27, 4-{4-[(R)-(6-cyclopropyl-imidazo[1,5-a]pyrazin-5-yl)-hydroxy-methyl]-[1,2,3]triazol-1-yl}-2-fluoro-phenol and 1-bromo-2-methylpropan-2-ol. Purification by preparative HPLC (basic conditions) gives 1-(4-{4-[(R)-(6-cyclopropyl-imidazo[1,5-a]pyrazin-5-yl)-hydroxy-methyl]-[1,2,3]triazol-1-yl}-2-fluoro-phenoxy)-2-methyl-propan-2-ol. LC-MS (QC): t_{R}=0.767 min, [M+H]⁺=439.3. ¹H NMR (500 MHz, DMSO) δ: 8.99 (d, J=0.6 Hz, 1H), 8.96 (s, 1H), 8.62 (s, 1H), 7.88 (dd, J1=11.9 Hz, J2=2.6 Hz, 1H), 7.77 (d, J=0.5 Hz, 1H), 7.70 (ddd, J1=8.9 Hz, J2=2.6 Hz, J3=1.5 Hz, 1H), 7.38 (t, J=9.1 Hz, 1H), 6.86 (d, J=3.8 Hz, 1H), 6.79 (d, J=4.0 Hz, 1H), 4.71 (s, 1H), 3.87 (s, 2H), 2.47 (m, 1H), 1.23 (s, 6H), 1.06-1.08 (m, 1H), 0.94-1.00 (m, 3H).

### Example 30: (R)-(6-Chloro-imidazo[1,5-a]pyridin-5-yl)-[1-(4-methoxy-phenyl)-1H-[1,2,3]triazol-4-yl]-methanol

Prepared following the procedure described for Example 1 using Intermediate Ca, (S)-1-(6-chloroimidazo[1,5-a]pyridin-5-yl)prop-2-yn-1-ol and 1-azido-4-methoxybenzene. Purification by preparative HPLC (basic conditions) gives (R)-(6-chloro-imidazo[1,5-a]pyridin-5-yl)-[1-(4-methoxy-phenyl)-1H-[1,2,3]triazol-4-yl]-methanol. LC-MS (QC): t_{R}=0.683 min, [M+H]⁺=356.1. ¹H NMR (500 MHz, DMSO) δ: 8.97 (s, 1H), 8.62 (s, 1H), 7.83 (d, J=8.2 Hz, 2H), 7.65 (d, J=9.3 Hz, 1H), 7.45-7.54 (m, 1H), 7.13 (d, J=8.2 Hz, 2H), 6.97 (d, J=3.0 Hz, 1H), 6.90 (d, J=9.4 Hz, 1H), 6.80 (s, 1H), 3.83 (s, 3H).

### Example 31: (R)-(6-Chloro-imidazo[1,5-a]pyridin-5-yl)-[1-(2,5-difluoro-4-methoxy-phenyl)-1H-[1,2,3]triazol-4-yl]-methanol

Prepared following the procedure described for Example 22 using Intermediate Ca, (S)-1-(6-chloroimidazo[1,5-a]pyridin-5-yl)prop-2-yn-1-ol and 1-azido-2,5-difluoro-4-methoxybenzene. Purification by preparative HPLC (basic conditions) gives (R)-(6-chloro-imidazo[1,5-a]pyridin-5-yl)-[1-(2,5-difluoro-4-methoxy-phenyl)-1H-[1,2,3]triazol-4-yl]-methanol. LC-MS (QC): t_{R}=0.737 min, [M+H]⁺=392.1. ¹H NMR (500 MHz, DMSO) δ: 8.79 (s, 1H), 8.59 (s, 1H), 7.84 (dd, J1=7.1 Hz, J2=11.2 Hz, 1H), 7.66 (d, J=9.5 Hz, 1H), 7.47-7.54 (m, 2H), 7.00 (d, J=4.4 Hz, 1H), 6.91 (d, J=9.5 Hz, 1H), 6.81 (d, J=4.1 Hz, 1H), 3.95 (s, 3H).

### Example 32: rac-[1-(2,5-Difluoro-4-methoxy-phenyl)-1H-[1,2,3]triazol-4-yl]-(6-ethyl-imidazo[1,5-a]pyrazin-5-yl)-methanol

Prepared following the procedure described for Example 22 using Intermediate F, rac-1-(6-ethylimidazo[1,5-a]pyrazin-5-yl)prop-2-yn-1-ol and 1-azido-2,5-difluoro-4-methoxybenzene. Purification by preparative HPLC (basic conditions) gives rac-[1-(2,5-difluoro-4-methoxy-phenyl)-1H-[1,2,3]triazol-4-yl]-(6-ethyl-imidazo[1,5-a]pyrazin-5-yl)-methanol. LC-MS (QC): t_{R}=0.729 min, [M+H]⁺=387.2.

### Example 33: (R)-[1-(3-Chloro-2-fluoro-4-methoxy-phenyl)-1H-[1,2,3]triazol-4-yl]-(6-cyclopropyl-imidazo[1,5-a]pyrazin-5-yl)-methanol

### Step 1: Preparation of 1-azido-3-chloro-2-fluoro-4-methoxybenzene

Prepared according to the procedure described for Example 7, step 1 using (3-chloro-2-fluoro-4-methoxyphenyl)boronic acid.

### Step 2

Prepared following the procedure described for Example 1 using Intermediate Ba, (S)-1-(6-cyclopropylimidazo[1,5-a]pyrazin-5-yl)prop-2-yn-1-ol and 1-azido-3-chloro-2-fluoro-4-methoxybenzene. Purification by prepHPLC (acidic conditions) to give (R)-[1-(3-chloro-2-fluoro-4-methoxy-phenyl)-1H-[1,2,3]triazol-4-yl]-(6-cyclopropyl-imidazo[1,5-a]pyrazin-5-yl)-methanol. LC-MS (QC): t_{R}=0.863; [M+H]⁺=415.2. ¹H NMR (500 MHz, DMSO) δ: 8.96 (s, 1H), 8.79 (d, J=1.1 Hz, 1H), 8.62 (s, 1H), 7.77-7.78 (m, 2H), 7.23 (dd, J1=9.3 Hz, J2=1.8 Hz, 1H), 6.89 (d, J=3.8 Hz, 1H), 6.81 (d, J=4.0 Hz, 1H), 3.99 (s, 3H), 2.47 (m, 1H), 1.05-1.08 (m, 1H), 0.93-1.01 (m, 3H).

### Example 34: rac-(6-Ethyl-imidazo[1,5-a]pyridin-5-yl)-[1-(4-methoxy-phenyl)-1H-[1,2,3]triazol-4-yl]-methanol

Prepared following the procedure described for Example 1 using Intermediate E, rac-1-(6-ethylimidazo[1,5-a]pyridin-5-yl)prop-2-yn-1-ol and 1-azido-4-methoxybenzene. Purification by preparative HPLC (basic conditions) gives rac-(6-ethyl-imidazo[1,5-a]pyridin-5-yl)-[1-(4-methoxy-phenyl)-1H-[1,2,3]triazol-4-yl]-methanol. LC-MS (QC): t_{R}=0.605 min, [M+H]⁺=350.2. ¹H NMR (500 MHz, DMSO) δ: 8.95 (d, 1H), 8.45-8.54 (m, 1H), 7.83 (m, 2H), 7.43-7.57 (m, 1H), 7.32 (s, 1H), 7.13 (m, 2H), 6.76-6.79 (m, 1H), 6.62 (m, 2H), 3.83 (s, 3H), 2.75-2.85 (m, 2H), 1.09-1.31 (m, 3H).

### Example 34a: (R)-(6-Ethyl-imidazo[1,5-a]pyridin-5-yl)-[1-(4-methoxy-phenyl)-1H-[1,2,3]triazol-4-yl]-methanol

Separation of rac-(6-ethyl-imidazo[1,5-a]pyridin-5-yl)-[1-(4-methoxy-phenyl)-1H-[1,2,3]triazol-4-yl]-methanol on chiral stationary phase. Method: Column: ChiralPak IH 30×250 mm, 5 μM; Detector Wavelength: 222 nM; Eluent: 50% CO₂ and 50% (MeCN/EtOH/DEA 50:50:0.1); Flow: 160.00 mL/min; BPR: 100 bar; Temperature: 40° C.; Injection volume: 1000 μl.

18 mg of the racemate are separated by the method described above to give:

7.7 mg of the R-enantiomer Example 34a and 9.6 mg of the S-enantiomer.

Example 34a: LC-MS (QC): t_{R}=0.606 min, [M+H]⁺=350.2.

### Example 35: rac-[1-(2,5-Difluoro-4-methoxy-phenyl)-1H-[1,2,3]triazol-4-yl]-(6-ethyl-imidazo[1,5-a]pyridin-5-yl)-methanol

Prepared following the procedure described for Example 22 using Intermediate E, rac-1-(6-ethylimidazo[1,5-a]pyridin-5-yl)prop-2-yn-1-ol and 1-azido-2,5-difluoro-4-methoxybenzene. Purification by preparative HPLC (basic conditions) gives rac-[1-(2,5-difluoro-4-methoxy-phenyl)-1H-[1,2,3]triazol-4-yl]-(6-ethyl-imidazo[1,5-a]pyridin-5-yl)-methanol. LC-MS (QC): t_{R}=0.641 min, [M+H]⁺=386.2. ¹H NMR (500 MHz, DMSO) δ: 8.74 (s, 1H), 8.33-8.53 (m, 1H), 7.82 (dd, J1=11.2 Hz, J2=7.1 Hz, 1H), 7.46-7.56 (m, 2H), 7.32 (s, 1H), 6.77 (d, J=9.2 Hz, 1H), 6.67 (d, J=3.8 Hz, 1H), 6.56-6.62 (m, 1H), 3.86-4.00 (m, 3H), 2.80 (q, J=7.5 Hz, 2H), 1.28 (t, J=7.5 Hz, 3H).

### Example 35a: (R)-[1-(2,5-Difluoro-4-methoxy-phenyl)-1H-[1,2,3]triazol-4-yl]-(6-ethyl-imidazo[1,5-a]pyridin-5-yl)-methanol

Separation of rac-[1-(2,5-difluoro-4-methoxy-phenyl)-1H-[1,2,3]triazol-4-yl]-(6-ethyl-imidazo[1,5-a]pyridin-5-yl)-methanol on chiral stationary phase. Method: Column: ChiralPak IH 30×250 mm, 5 μM; Detector Wavelength: 222 nM; Eluent: 55% CO₂ and 45% (MeCN/EtOH/DEA 50:50:0.1); Flow: 160.00 mL/min; BPR: 100 bar; Temperature: 40° C.; Injection volume: 1000 μl.

16 mg of the racemate are separated by the method described above to give:

8.6 mg of the R-enantiomer Example 35a and 5.5 mg of the S-enantiomer.

Example 35a: LC-MS (QC): t_{R}=0.641 min, [M+H]⁺=386.2.

### Example 36: (R)-(6-Cyclopropyl-imidazo[1,5-a]pyrazin-5-yl)-{1-[3-fluoro-4-(oxetan-3-ylmethoxy)-phenyl]-1H-[1,2,3]triazol-4-yl}-methanol

Prepared following the procedure described for Example 16 using Example 27, 4-{4-[(R)-(6-cyclopropyl-imidazo[1,5-a]pyrazin-5-yl)-hydroxy-methyl]-[1,2,3]triazol-1-yl}-2-fluoro-phenol and 3-(bromomethyl)oxetane. Purification by preparative HPLC (acidic conditions) gives (R)-(6-cyclopropyl-imidazo[1,5-a]pyrazin-5-yl)-{1-[3-fluoro-4-(oxetan-3-ylmethoxy)-phenyl]-1H-[1,2,3]triazol-4-yl}-methanol. LC-MS (QC): t_{R}=0.748 min, [M+H]⁺=437.2. ¹H NMR (500 MHz, DMSO) δ: 9.00 (d, J=0.5 Hz, 1H), 8.96 (s, 1H), 8.62 (s, 1H), 7.90 (dd, J1=2.6 Hz, J2=12.0 Hz, 1H), 7.77 (d, J=0.4 Hz, 1H), 7.74 (ddd, J1=8.9 Hz, J2=2.6 Hz, J3=1.5 Hz, 1H), 7.43 (t, J=9.1 Hz, 1H), 6.86 (d, J=3.8 Hz, 1H), 6.80 (d, J=4.0 Hz, 1H), 4.73 (dd, J1=6.1 Hz, J2=7.9 Hz, 2H), 4.45 (t, J=6.1 Hz, 2H), 4.37 (d, J=6.7 Hz, 2H), 3.42-3.46 (m, 1H), 2.40-2.47 (m, 1H), 1.06-1.09 (m, 1H), 0.94-1.00 (m, 3H).

### Example 37: rac-(6-Chloro-imidazo[1,5-a]pyridin-5-yl)-[5-ethyl-1-(4-methoxy-phenyl)-1H-[1,2,3]triazol-4-yl]-methanol

### Step 1: preparation of ethyl 2-diazo-3-oxopentanoate

A solution of ethyl propionylacetate (4474 mg, 29.8 mmol, 1 eq) and 4-acetamidobenzenesulfonyl azide (7748 mg, 31.3 mmol, 1.05 eq) in MeCN (150 mL) is stirred under argon atmosphere at RT. All materials are dissolved completely, TEA (4.35 mL, 31.3 mmol, 1.05 eq) is added to the reaction which is stirred at room temperature overnight. The white precipitate is filtered, washed with CH₂Cl₂, and the filtrate concentrated under reduced pressure to give the title product.

### Step 2: Preparation of ethyl 5-ethyl-1-(4-methoxyphenyl)-1H-1,2,3-triazole-4-carboxylate

To a solution of ethyl 2-diazo-3-oxopentanoate (478 mg, 2.81 mmol) in DMF (6.12 mL) under argon atmosphere at RT are successively added 4-amino-anisol (0.363 mL, 3.09 mmol, 1.1 eq) and titanium(IV) chloride (˜1.0 M in toluene, 2.81 mL, 2.81 mmol, 1 eq). The reaction mixture is stirred overnight at 80° C. The slurry is diluted with water and EtOAc, and filtered. After separation the aqueous phase is extracted twice with EtOAc. The combined organic extracts are washed with water and brine, dried over MgSO₄, filtered and contrated under reduced pressure. Purification by silicagel flash chromatography (Hept/EtOAc) gives the title compound. LC-MS (acidic): t_{R}=0.82 min, [M+H]⁺=276.02.

### Step 3: Preparation of (5-ethyl-1-(4-methoxyphenyl)-1H-1,2,3-triazol-4-yl)methanol

Prepared following the procedure described for Example 20, step 2, using ethyl 5-ethyl-1-(4-methoxyphenyl)-1H-1,2,3-triazole-4-carboxylate. LC-MS (acidic): t_{R}=0.67 min, [M+H]⁺=234.41.

### Step 4: Preparation of 5-ethyl-1-(4-methoxyphenyl)-1H-1,2,3-triazole-4-carbaldehyde

Prepared following the procedure described for Example 20, step 3, using (5-ethyl-1-(4-methoxyphenyl)-1H-1,2,3-triazol-4-yl)methanol. LC-MS (acidic): t_{R}=0.84 min, [M+H]⁺=232.03.

### Step 4: Preparation of rac-(6-chloro-3-(ethylthio)imidazo[1,5-a]pyridin-5-yl)(5-ethyl-1-(4-methoxyphenyl)-1H-1,2,3-triazol-4-yl)methanol

Prepared following the procedure described for Reference example 2 using 6-chloro-3-(ethylthio)imidazo[1,5-a]pyridine and 5-ethyl-1-(4-methoxyphenyl)-1H-1,2,3-triazole-4-carbaldehyde. LC-MS (acidic): t_{R}=0.83 min, [M+H]⁺=444.17.

### Step 5: Preparation of rac-(6-chloro-imidazo[1,5-a]pyridin-5-yl)-[5-ethyl-1-(4-methoxy-phenyl)-1H-[1,2,3]triazol-4-yl]-methanol

Prepared following the procedure described for Reference example 2 using rac-(6-chloro-3-(ethylthio)imidazo[1,5-a]pyridin-5-yl)(5-ethyl-1-(4-methoxyphenyl)-1H-1,2,3-triazol-4-yl)methanol. Purification by preparative HPLC (basic conditions) gives rac-(6-chloro-imidazo[1,5-a]pyridin-5-yl)-[5-ethyl-1-(4-methoxy-phenyl)-1H-[1,2,3]triazol-4-yl]-methanol. LC-MS (QC): t_{R}=0.748 min, [M+H]⁺=384.2.

### Example 38: rac-(6-Chloro-imidazo[1,5-a]pyridin-5-yl)-(5-methyl-1-phenyl-1H-[1,2,3]triazol-4-yl)-methanol

### Step 1: Preparation of rac-(6-chloro-3-(ethylthio)imidazo[1,5-a]pyridin-5-yl)(5-methyl-1-phenyl-1H-1,2,3-triazol-4-yl)methanol

Prepared following the procedure described for Reference example 2 using 6-chloro-3-(ethylthio)imidazo[1,5-a]pyridine and 5-methyl-1-phenyl-1H-1,2,3-triazole-4-carbaldehyde. LC-MS (acidic): t_{R}=0.75 min, [M+H]⁺=400.23.

### Step 2: Preparation of rac-(6-chloro-imidazo[1,5-a]pyridin-5-yl)-(5-methyl-1-phenyl-1H-[1,2,3]triazol-4-yl)-methanol

Prepared following the procedure described for Reference example 2 using rac-(6-chloro-3-(ethylthio)imidazo[1,5-a]pyridin-5-yl)(5-methyl-1-phenyl-1H-1,2,3-triazol-4-yl)methanol. Purification by preparative HPLC (basic conditions) gives rac-(6-chloro-imidazo[1,5-a]pyridin-5-yl)-(5-methyl-1-phenyl-1H-[1,2,3]triazol-4-yl)-methanol. LC-MS (QC): t_{R}=0.651 min, [M+H]⁺=340.3. ¹H NMR (500 MHz, DMSO) δ: 8.74 (s, 1H), 7.61-7.66 (m, 6H), 7.50 (s, 1H), 6.87 (d, J=9.5 Hz, 1H), 6.80 (d, J=4.3 Hz, 1H), 6.74 (d, J=4.2 Hz, 1H), 2.46 (s, 3H).

### Example 39: rac-[1-(5-Chloro-2-fluoro-4-methoxy-phenyl)-5-methyl-1H-[1,2,3]triazol-4-yl]-(6-chloro-imidazo[1,5-a]pyridin-5-yl)-methanol

### Step 1: preparation of ethyl 1-(5-chloro-2-fluoro-4-methoxyphenyl)-1H-1,2,3-triazole-4-carboxylate

A solution of 1-azido-5-chloro-2-fluoro-4-methoxybenzene (248 mg, 1.23 mmol, 1 eq) and ethyl propiolate (133 mg, 0.449 mmol, 1.5 eq) in DMF (1.2 mL) is cooled to 0° C. The catalyst CuSO₄/sodium ascorbate/BPDS (prepared by adding BPDS (4.1 mg, 6.98 10E-3 mmol, 5.7E-3 eq) in water (658 uL) and DMF (16 □L) to a solution of CuSO₄ (1.7 mg, 6.61 10E-3 mmol, 5.4 10E-3 eq) and sodium ascorbate (1.7 mg, 8.24 10E-3 mmol, 6.7 10E-3 eq) in water (205 □L)) is added at 0° C., and the mixture stirred at RT for 2 d. The mixture is filtered and washed with water to give the title product as a yellow solid (307 mg, 83%). LC-MS (acidic): t_{R}=0.92 min, [M+H]⁺=300.10.

### Step 2: Preparation of (1-(5-chloro-2-fluoro-4-methoxyphenyl)-1H-1,2,3-triazol-4-yl)methanol

Prepared following the procedure described for Example 20, step 2, using ethyl 1-(5-chloro-2-fluoro-4-methoxyphenyl)-1H-1,2,3-triazole-4-carboxylate. LC-MS (acidic): t_{R}=0.69 min, [M+H]⁺=258.12.

### Step 3: Preparation of 1-(5-chloro-2-fluoro-4-methoxyphenyl)-1H-1,2,3-triazole-4-carbaldehyde

To a solution of (1-(5-chloro-2-fluoro-4-methoxyphenyl)-1H-1,2,3-triazol-4-yl)methanol (263 mg, 1 mmol, 1 eq) in CH₂Cl₂ (11 mL) is added MnO₂ (878 mg, 10.1 mmol, 10.1 eq). The mixture is stirred at RT until completion of the reaction. The mixture is filtered and concentrated under reduced pressure. Purification by FC (Hept/EtOAc) gives the title product as a light brown solid (137 mg, 54%). LC-MS (acidic): t_{R}=0.83 min, [M+H]+=256.22.

### Step 4: Preparation of rac-(1-(5-chloro-2-fluoro-4-methoxyphenyl)-5-methyl-1H-1,2,3-triazol-4-yl)(6-chloro-3-(ethylthio)imidazo[1,5-a]pyridin-5-yl)methanol

Prepared following the procedure described for Reference example 2 using 6-chloro-3-(ethylthio)imidazo[1,5-a]pyridine and 1-(5-chloro-2-fluoro-4-methoxyphenyl)-1H-1,2,3-triazole-4-carbaldehyde. LC-MS (acidic): t_{R}=0.86 min, [M+H]⁺=482.08.

### Step 5: preparation of rac-[1-(5-chloro-2-fluoro-4-methoxy-phenyl)-5-methyl-1H-[1,2,3]triazol-4-yl]-(6-chloro-imidazo[1,5-a]pyridin-5-yl)-methanol

Prepared following the procedure described for Reference example 2 using rac-(1-(5-chloro-2-fluoro-4-methoxyphenyl)-5-methyl-1H-1,2,3-triazol-4-yl)(6-chloro-3-(ethylthio)imidazo[1,5-a]pyridin-5-yl)methanol. Purification by preparative HPLC (basic conditions) gives rac-[1-(5-chloro-2-fluoro-4-methoxy-phenyl)-5-methyl-1H-[1,2,3]triazol-4-yl]-(6-chloro-imidazo[1,5-a]pyridin-5-yl)-methanol. LC-MS (QC): t_{R}=0.782 min, [M+H]⁺=422.3. ¹H NMR (500 MHz, DMSO) δ: 8.70 (s, 1H), 7.93 (d, J=7.8 Hz, 1H), 7.65 (d, J=9.5 Hz, 1H), 7.50 (m, 2H), 6.87 (d, J=9.5 Hz, 1H), 6.84 (d, J=4.4 Hz, 1H), 6.75 (d, J=4.4 Hz, 1H), 3.98 (s, 3H), 2.35 (s, 3H).

### Example 40: (R)-(6-Cyclopropyl-imidazo[1,5-a]pyrazin-5-yl)-[1-(4-pyrrolidin-1-yl-phenyl)-1H-[1,2,3]triazol-4-yl]-methanol

### Step 1: Preparation of 1-(4-azidophenyl)pyrrolidine

Prepared according to the procedure described for Example 7, step 1 using (4-(pyrrolidin-1-yl)phenyl)boronic acid HCl.

### Step 2: Preparation of (R)-(6-cyclopropyl-imidazo[1,5-a]pyrazin-5-yl)-[1-(4-pyrrolidin-1-yl-phenyl)-1H-[1,2,3]triazol-4-yl]-methanol

Prepared following the procedure described for Example 7 using Intermediate Ba, (S)-1-(6-cyclopropylimidazo[1,5-a]pyrazin-5-yl)prop-2-yn-1-ol and 1-(4-azidophenyl)pyrrolidine. Purification by prepHPLC (basic conditions) to give (R)-(6-cyclopropyl-imidazo[1,5-a]pyrazin-5-yl)-[1-(4-pyrrolidin-1-yl-phenyl)-1H-[1,2,3]triazol-4-yl]-methanol. LC-MS (QC): t_{R}=0.933 min; [M+H]⁺=402.4. ¹H NMR (500 MHz, DMSO) 8.95 (s, 1H), 8.82 (d, 1H), 8.65 (s, 1H), 7.77 (s, 1H), 7.65 (d, J=9.0 Hz, 2H), 6.84 (d, J=3.9 Hz, 1H), 6.73 (d, J=4.0 Hz, 1H), 6.65 (d, J=9.1 Hz, 2H), 3.28 (t, J=6.6 Hz, 4H), 2.45-2.47 (m, 1H), 1.98 (m, 4H), 1.05-1.08 (m, 1H), 0.93-1.00 (m, 3H).

### Example 41: (R)-[1-(4-Amino-phenyl)-1H-[1,2,3]triazol-4-yl]-(6-cyclopropyl-imidazo[1,5-a]pyrazin-5-yl)-methanol

Prepared following the procedure described for Example 7 using Intermediate Ba, (S)-1-(6-cyclopropylimidazo[1,5-a]pyrazin-5-yl)prop-2-yn-1-ol and 4-azidoaniline. Purification by prepHPLC (basic conditions) to give (R)-[1-(4-amino-phenyl)-1H-[1,2,3]triazol-4-yl]-(6-cyclopropyl-imidazo[1,5-a]pyrazin-5-yl)-methanol. LC-MS (QC): t_{R}=0.529 min; [M+H]⁺=348.3. ¹H NMR (500 MHz, DMSO) δ: 8.95 (s, 1H), 8.75 (d, 1H), 8.64 (s, 1H), 7.76 (d, 1H), 7.48 (d, J=8.8 Hz, 2H), 6.83 (d, J=3.8 Hz, 1H), 6.72 (d, J=4.0 Hz, 1H), 6.67 (d, J=8.9 Hz, 2H), 5.49 (s, 2H), 2.44-2.48 (m, 1H), 1.05-1.07 (m, 1H), 0.93-0.99 (m, 3H).

### Example 42: (R)-(6-Cyclopropyl-imidazo[1,5-a]pyrazin-5-yl)-[1-(4-methylamino-phenyl)-1H-[1,2,3]triazol-4-yl]-methanol

### Step 1: Preparation of 4-azido-N-methylaniline

Prepared according to the procedure described for Example 7, step 1 using N-methyl-4-(4,4,5,5-tetramethyl-1,3,2-dioxaborolan-2-yl)aniline.

### Step 2: Preparation of (R)-(6-cyclopropyl-imidazo[1,5-a]pyrazin-5-yl)-[1-(4-methylamino-phenyl)-1H-[1,2,3]triazol-4-yl]-methanol

Prepared following the procedure described for Example 7 using Intermediate Ba, (S)-1-(6-cyclopropylimidazo[1,5-a]pyrazin-5-yl)prop-2-yn-1-ol and 4-azido-N-methylaniline. Purification by prepHPLC (basic conditions) to give (R)-(6-cyclopropyl-imidazo[1,5-a]pyrazin-5-yl)-[1-(4-methylamino-phenyl)-1H-[1,2,3]triazol-4-yl]-methanol. LC-MS (QC): t_{R}=0.656 min; [M+H]⁺=362.3. ¹H NMR (500 MHz, DMSO) δ: 8.95 (s, 1H), 8.78 (d, 1H), 8.64 (s, 1H), 7.77 (s, 1H), 7.57 (d, J=8.9 Hz, 2H), 6.83 (d, J=3.8 Hz, 1H), 6.72 (d, J=4.0 Hz, 1H), 6.65 (d, J=9.0 Hz, 2H), 6.09 (q, J=5.0 Hz, 1H), 2.72 (d, J=5.0 Hz, 3H), 2.46 (m, 1H), 1.05-1.08 (m, 1H), 0.93-0.99 (m, 3H).

### Example 43: rac-2-Chloro-4-{4-[(6-chloro-imidazo[1,5-a]pyridin-5-yl)-hydroxy-methyl]-5-methyl-[1,2,3]triazol-1-yl}-phenol

### Step 1: Preparation of 4-azido-2-chloro-1-(methoxymethoxy)benzene

Prepared according to the procedure described for Example 7, step 1 using (3-chloro-4-(methoxymethoxy)phenyl)boronic acid.

### Step 2: Preparation of ethyl 1-(3-chloro-4-(methoxymethoxy)phenyl)-5-methyl-1H-1,2,3-triazole-4-carboxylate

Prepared following the procedure described for Example 20, step 1, using 4-azido-2-chloro-1-(methoxymethoxy)benzene. LC-MS (acidic): t_{R}=0.92 min, [M+H]⁺=326.26.

### Step 3: Preparation of (1-(3-chloro-4-(methoxymethoxy)phenyl)-5-methyl-1H-1,2,3-triazol-4-yl)methanol

Prepared following the procedure described for Example 20, step 2, using ethyl 1-(3-chloro-4-(methoxymethoxy)phenyl)-5-methyl-1H-1,2,3-triazole-4-carboxylate. LC-MS (acidic): t_{R}=0.70 min, [M+H]⁺=284.18.

### Step 4: Preparation of 1-(3-chloro-4-(methoxymethoxy)phenyl)-5-methyl-1H-1,2,3-triazole-4-carbaldehyde

Prepared following the procedure described for Example 20, step 3, using (1-(3-chloro-4-(methoxymethoxy)phenyl)-5-methyl-1H-1,2,3-triazol-4-yl)methanol. LC-MS (acidic): t_{R}=0.85 min, [M+H]⁺=282.19.

### Step 5: Preparation of rac-(6-chloro-3-(ethylthio)imidazo[1,5-a]pyridin-5-yl)(1-(3-chloro-4-(methoxymethoxy)phenyl)-5-methyl-1H-1,2,3-triazol-4-yl)methanol

Prepared following the procedure described for Reference example 2 using 6-chloro-3-(ethylthio)imidazo[1,5-a]pyridine and 1-(3-chloro-4-(methoxymethoxy)phenyl)-5-methyl-1H-1,2,3-triazole-4-carbaldehyde. LC-MS (acidic): t_{R}=0.85 min, [M+H]⁺=494.24.

### Step 6: Preparation of rac-(1-(3-chloro-4-(methoxymethoxy)phenyl)-5-methyl-1H-1,2,3-triazol-4-yl)(6-chloroimidazo[1,5-a]pyridin-5-yl)methanol

Prepared following the procedure described for Reference example 2 using rac-(6-chloro-3-(ethylthio)imidazo[1,5-a]pyridin-5-yl)(1-(3-chloro-4-(methoxymethoxy)phenyl)-5-methyl-1H-1,2,3-triazol-4-yl)methanol. Purification by preparative HPLC (basic conditions) gives rac-(1-(3-chloro-4-(methoxymethoxy)phenyl)-5-methyl-1H-1,2,3-triazol-4-yl)(6-chloroimidazo[1,5-a]pyridin-5-yl)methanol. LC-MS (acidic): t_{R}=0.73 min, [M+H]⁺=433.87.

### Step 7: preparation of rac-2-chloro-4-{4-[(6-chloro-imidazo[1,5-a]pyridin-5-yl)-hydroxy-methyl]-5-methyl-[1,2,3]triazol-1-yl}-phenol

To a solution of rac-(1-(3-chloro-4-(methoxymethoxy)phenyl)-5-methyl-1H-1,2,3-triazol-4-yl)(6-chloroimidazo[1,5-a]pyridin-5-yl)methanol in EtOAc is added HCl in dioxane (4M, 4.5 eq) and the white suspension is stirred at RT until completion of the reaction. The suspension is filtered and concentrated under reduced pressure to give rac-2-chloro-4-{4-[(6-chloro-imidazo[1,5-a]pyridin-5-yl)-hydroxy-methyl]-5-methyl-[1,2,3]triazol-1-yl}-phenol. LC-MS (QC): t_{R}=0.639 min, [M+H]⁺=390.3.

### Example 44: (R)-(6-Cyclopropyl-imidazo[1,5-a]pyrazin-5-yl)-[1-(4-dimethylamino-phenyl)-1H-[1,2,3]triazol-4-yl]-methanol

Prepared following the procedure described for Example 7 using Intermediate Ba, (S)-1-(6-cyclopropylimidazo[1,5-a]pyrazin-5-yl)prop-2-yn-1-ol and 4-azido-N,N-dimethylaniline. Purification by prepHPLC (basic conditions) to give (R)-(6-cyclopropyl-imidazo[1,5-a]pyrazin-5-yl)-[1-(4-dimethylamino-phenyl)-1H-[1,2,3]triazol-4-yl]-methanol. LC-MS (QC): t_{R}=0.771 min; [M+H]⁺=376.3. ¹H NMR (500 MHz, DMSO) δ: 8.95 (s, 1H), 8.84 (d, 1H), 8.65 (s, 1H), 7.77 (s, 1H), 7.67 (d, J=9.1 Hz, 2H), 6.84 (m, 3H), 6.74 (d, J=4.0 Hz, 1H), 2.97 (s, 6H), 2.45-2.47 (m, 1H), 1.06-1.08 (m, 1H), 0.93-0.99 (m, 3H).

### Example 45: (R)-(6-Cyclopropyl-imidazo[1,5-a]pyrazin-5-yl)-{1-[4-(2-oxa-6-aza-spiro[3.3]hept-6-yl)-phenyl]-1H-[1,2,3]triazol-4-yl}-methanol

### Step 1: Preparation of 6-(4-nitrophenyl)-2-oxa-6-azaspiro[3.3]heptane

A solution of 1-fluoro-4-nitrobenzene (388 mg, 2.72 mmol, 1 eq) and DIPEA (1.02 mL, 5.96 mmol, 2.19 eq) in CH₃CN (3 mL) is treated with 2-oxa-6-aza-spiro-3-3-heptane (278 mg, 2.72 mmol, 1 eq) at RT. The mixture is then stirred at 75° C. for 24 h. More 2-oxa-6-aza-spiro-3-3-heptane (278 mg, 2.72 mmol, 1 eq) is added at RT and the mixture stirred at 75° C. overnight. The mixture is cooled to RT and concentrated under reduced pressure. The residue is suspended in DMF and filtered to get the title product as a yellow solid (453 mg, 76%). LC-MS (acidic): tᵣ=0.80 min, [M+H]⁺=221.29.

### Step 2: Preparation of 4-(2-oxa-6-azaspiro[3.3]heptan-6-yl)aniline

A solution of 6-(4-nitrophenyl)-2-oxa-6-azaspiro[3.3]heptane (453 mg, 2.04 mmol, 1 eq) in MeOH (8 mL) is degassed three times and inerted with N₂. Then Pd/C 10% (71 mg) is added at RT. The mixture is degassed, placed under hydrogen atmosphere and stirred at RT for 4 h. The mixture is filtered through a Whatman 0.45 μm glass microfiber filter and concentrated under reduced pressure to get the title product as a purple solid (370 mg, 95%). LC-MS (acidic): tᵣ=0.37 min, [M+H]⁺=190.31.

### Step 3: Preparation of 6-(4-azidophenyl)-2-oxa-6-azaspiro[3.3]heptane

Prepared according to the procedure described for Example 8, step 1 using 4-(2-oxa-6-azaspiro[3.3]heptan-6-yl)aniline.

### Step 4: preparation of (R)-(6-cyclopropyl-imidazo[1,5-a]pyrazin-5-yl)-{1-[4-(2-oxa-6-aza-spiro[3.3]hept-6-yl)-phenyl]-1H-[1,2,3]triazol-4-yl}-methanol

Prepared following the procedure described for Example 7 using Intermediate Ba, (S)-1-(6-cyclopropylimidazo[1,5-a]pyrazin-5-yl)prop-2-yn-1-ol and 6-(4-azidophenyl)-2-oxa-6-azaspiro[3.3]heptane. Purification by prepHPLC (basic conditions) to give (R)-(6-cyclopropyl-imidazo[1,5-a]pyrazin-5-yl)-{1-[4-(2-oxa-6-aza-spiro[3.3]hept-6-yl)-phenyl]-1H-[1,2,3]triazol-4-yl}-methanol. LC-MS (QC): t_{R}=0.700 min; [M+H]⁺=430.4. ¹H NMR (500 MHz, DMSO) δ: 8.95 (s, 1H), 8.84 (s, 1H), 8.58-8.72 (m, 1H), 7.73-7.87 (m, 1H), 7.67 (d, J=8.9 Hz, 2H), 6.84 (d, J=3.9 Hz, 1H), 6.74 (d, J=4.0 Hz, 1H), 6.57 (d, J=8.9 Hz, 2H), 4.73 (s, 4H), 4.05 (s, 4H), 2.43-2.48 (m, 1H), 1.05-1.09 (m, 1H), 0.93-1.00 (m, 3H).

### Example 46: (R)-(6-Cyclopropyl-imidazo[1,5-a]pyrazin-5-yl)-{1-[4-(6-oxa-1-aza-spiro[3.3]hept-1-yl)-phenyl]-1H-[1,2,3]triazol-4-yl}-methanol

### Step 1: Preparation of 1-(4-nitrophenyl)-6-oxa-1-azaspiro[3.3]heptane

Prepared according to the procedure described for Example 45, step 1 using 6-oxa-1-azaspiro[3.3]heptane. LC-MS (acidic): tᵣ=0.75 min, [M+H]⁺=221.11.

### Step 2: Preparation of 4-(6-oxa-1-azaspiro[3.3]heptan-1-yl)aniline

Prepared according to the procedure described for Example 45, step 2 using 1-(4-nitrophenyl)-6-oxa-1-azaspiro[3.3]heptane. LC-MS (acidic): tᵣ=0.36 min, [M+H]⁺=190.25.

### Step 3: Preparation of 1-(4-azidophenyl)-6-oxa-1-azaspiro[3.3]heptane

Prepared according to the procedure described for Example 8, step 1 using 4-(6-oxa-1-azaspiro[3.3]heptan-1-yl)aniline.

### Step 4: preparation of (R)-(6-cyclopropyl-imidazo[1,5-a]pyrazin-5-yl)-{1-[4-(6-oxa-1-aza-spiro[3.3]hept-1-yl)-phenyl]-1H-[1,2,3]triazol-4-yl}-methanol

Prepared following the procedure described for Example 7 using Intermediate Ba, (S)-1-(6-cyclopropylimidazo[1,5-a]pyrazin-5-yl)prop-2-yn-1-ol and 1-(4-azidophenyl)-6-oxa-1-azaspiro[3.3]heptane. Purification by prepHPLC (basic conditions) to give (R)-(6-cyclopropyl-imidazo[1,5-a]pyrazin-5-yl)-{1-[4-(6-oxa-1-aza-spiro[3.3]hept-1-yl)-phenyl]-1H-[1,2,3]triazol-4-yl}-methanol. LC-MS (QC): t_{R}=0.755 min; [M+H]⁺=430.4. ¹H NMR (500 MHz, DMSO) δ: 8.96 (s, 1H), 8.86 (d, 1H), 8.64 (s, 1H), 7.75 (m, 3H), 6.84 (d, J=9.0 Hz, 3H), 6.76 (s, 1H), 5.10 (d, J=7.8 Hz, 2H), 4.72 (d, J=8.0 Hz, 2H), 3.69 (t, J=6.9 Hz, 2H), 2.54 (m, 2H), 2.46 (m, 1H), 1.06-1.08 (m, 1H), 0.94-1.00 (m, 3H).

### Example 47: (R)-(6-Cyclopropyl-imidazo[1,5-a]pyrazin-5-yl)-[1-(1-methyl-2,3-dihydro-1H-indol-5-yl)-1H-[1,2,3]triazol-4-yl]-methanol

### Step 1: Preparation of 5-azido-1-methylindoline

Prepared according to the procedure described for Example 7, step 1 using 1-methyl-5-(4,4,5,5-tetramethyl-1,3,2-dioxaborolan-2-yl)indoline.

### Step 2: Preparation of (R)-(6-cyclopropyl-imidazo[1,5-a]pyrazin-5-yl)-[1-(1-methyl-2,3-dihydro-1H-indol-5-yl)-1H-[1,2,3]triazol-4-yl]-methanol

Prepared following the procedure described for Example 7 using Intermediate Ba, (S)-1-(6-cyclopropylimidazo[1,5-a]pyrazin-5-yl)prop-2-yn-1-ol and 5-azido-1-methylindoline. Purification by prepHPLC (basic conditions) to give (R)-(6-cyclopropyl-imidazo[1,5-a]pyrazin-5-yl)-[1-(1-methyl-2,3-dihydro-1H-indol-5-yl)-1H-[1,2,3]triazol-4-yl]-methanol. LC-MS (QC): t_{R}=0.793 min; [M+H]⁺=388.4.

### Example 48: rac-4-{4-[(6-Chloro-imidazo[1,5-a]pyridin-5-yl)-hydroxy-methyl]-5-methyl-[1,2,3]triazol-1-yl}-2-fluoro-phenol

### Step 1: Preparation of 4-azido-2-fluoro-1-(methoxymethoxy)benzene

Prepared according to the procedure (3-fluoro-4-(methoxymethoxy)phenyl)boronic acid.

### Step 2: Preparation of ethyl 1-(3-fluoro-4-(methoxymethoxy)phenyl)-5-methyl-1H-1,2,3-triazole-4-carboxylate

Prepared following the procedure described for Example 20, step 1, using 4-azido-2-fluoro-1-(methoxymethoxy)benzene. LC-MS (acidic): t_{R}=0.88 min, [M+H]⁺=310.21.

### Step 3: Preparation of (1-(3-fluoro-4-(methoxymethoxy)phenyl)-5-methyl-1H-1,2,3-triazol-4-yl)methanol

Prepared following the procedure described for Example 20, step 2, using ethyl 1-(3-fluoro-4-(methoxymethoxy)phenyl)-5-methyl-1H-1,2,3-triazole-4-carboxylate. LC-MS (acidic): t_{R}=0.65 min, [M+H]⁺=268.30.

### Step 4: Preparation of 1-(3-fluoro-4-(methoxymethoxy)phenyl)-5-methyl-1H-1,2,3-triazole-4-carbaldehyde

Prepared following the procedure described for Example 20, step 3, using (1-(3-fluoro-4-(methoxymethoxy)phenyl)-5-methyl-1H-1,2,3-triazol-4-yl)methanol. LC-MS (acidic): t_{R}=0.81 min, [M+H]⁺=266.26.

### Step 5: Preparation of rac-(6-chloro-3-(ethylthio)imidazo[1,5-a]pyridin-5-yl)(1-(3-fluoro-4-(methoxymethoxy)phenyl)-5-methyl-1H-1,2,3-triazol-4-yl)methanol

Prepared following the procedure described for Reference example 2 using 6-chloro-3-(ethylthio)imidazo[1,5-a]pyridine and 1-(3-fluoro-4-(methoxymethoxy)phenyl)-5-methyl-1H-1,2,3-triazole-4-carbaldehyde. LC-MS (acidic): t_{R}=0.81 min, [M+H]⁺=478.24.

### Step 6: Preparation of rac-(6-chloroimidazo[1,5-a]pyridin-5-yl)(1-(3-fluoro-4-(methoxymethoxy)phenyl)-5-methyl-1H-1,2,3-triazol-4-yl)methanol

Prepared following the procedure described for Reference example 2 using rac-(6-chloro-3-(ethylthio)imidazo[1,5-a]pyridin-5-yl)(1-(3-fluoro-4-(methoxymethoxy)phenyl)-5-methyl-1H-1,2,3-triazol-4-yl)methanol. Purification by preparative HPLC (basic conditions) gives rac-(6-chloroimidazo[1,5-a]pyridin-5-yl)(1-(3-fluoro-4-(methoxymethoxy)phenyl)-5-methyl-1H-1,2,3-triazol-4-yl)methanol. LC-MS (acidic): t_{R}=0.69 min, [M+H]⁺=418.04.

### Step 7: preparation of rac-4-{4-[(6-chloro-imidazo[1,5-a]pyridin-5-yl)-hydroxy-methyl]-5-methyl-[1,2,3]triazol-1-yl}-2-fluoro-phenol

To a solution of rac-(6-chloroimidazo[1,5-a]pyridin-5-yl)(1-(3-fluoro-4-(methoxymethoxy)phenyl)-5-methyl-1H-1,2,3-triazol-4-yl)methanol in EtOAc is added HCl in dioxane (4M, 2 eq) and the white suspension is stirred at RT until completion of the reaction. The suspension is filtered and concentrated under reduced pressure to give rac-4-{4-[(6-chloro-imidazo[1,5-a]pyridin-5-yl)-hydroxy-methyl]-5-methyl-[1,2,3]triazol-1-yl}-2-fluoro-phenol. LC-MS (QC): t_{R}=0.581 min, [M+H]⁺=374.3.

### Example 49: rac-4-(2-Chloro-4-{4-[(6-chloro-imidazo[1,5-a]pyridin-5-yl)-hydroxy-methyl]-5-methyl-[1,2,3]triazol-1-yl}-phenoxy)-2-methyl-butan-2-ol

Prepared following the procedure described for Example 16 using Example 43, rac-2-chloro-4-{4-[(6-chloro-imidazo[1,5-a]pyridin-5-yl)-hydroxy-methyl]-5-methyl-[1,2,3]triazol-1-yl}-phenol and 4-bromo-2-methylbutan-2-ol. Purification by preparative HPLC (basic conditions) gives rac-4-(2-chloro-4-{4-[(6-chloro-imidazo[1,5-a]pyridin-5-yl)-hydroxy-methyl]-5-methyl-[1,2,3]triazol-1-yl}-phenoxy)-2-methyl-butan-2-ol. LC-MS (QC): t_{R}=0.790 min, [M+H]⁺=476.2. ¹H NMR (500 MHz, DMSO) δ: 8.72 (s, 1H), 7.76 (d, J=2.6 Hz, 1H), 7.65 (d, J=9.5 Hz, 1H), 7.55 (dd, J₁=2.6 Hz, J₂=8.8 Hz, 1H), 7.49 (s, 1H), 7.37 (d, J=8.9 Hz, 1H), 6.86 (d, J=9.5 Hz, 1H), 6.78 (d, J=4.4 Hz, 1H), 6.72 (d, J=4.5 Hz, 1H), 4.43 (s, 1H), 4.27 (t, J=6.9 Hz, 2H), 2.37-2.44 (m, 3H), 1.91 (t, J=6.9 Hz, 2H), 1.20 (s, 6H).

### Example 50: rac-4-(4-{4-[(6-Chloro-imidazo[1,5-a]pyridin-5-yl)-hydroxy-methyl]-5-methyl-[1,2,3]triazol-1-yl}-2-fluoro-phenoxy)-2-methyl-butan-2-ol

Prepared following the procedure described for Example 16 using Example 48, rac-4-{4-[(6-chloro-imidazo[1,5-a]pyridin-5-yl)-hydroxy-methyl]-5-methyl-[1,2,3]triazol-1-yl}-2-fluoro-phenol and 4-bromo-2-methylbutan-2-ol. Purification by preparative HPLC (basic conditions) gives rac-4-(4-{4-[(6-chloro-imidazo[1,5-a]pyridin-5-yl)-hydroxy-methyl]-5-methyl-[1,2,3]triazol-1-yl}-2-fluoro-phenoxy)-2-methyl-butan-2-ol. LC-MS (QC): t_{R}=0.732 min, [M+H]⁺=460.3. ¹H NMR (500 MHz, DMSO) δ: 8.73 (s, 1H), 7.65 (d, J=9.5 Hz, 1H), 7.60-7.63 (m, 1H), 7.49 (s, 1H), 7.39-7.42 (m, 2H), 6.86 (d, J=9.6 Hz, 1H), 6.78 (d, J=4.3 Hz, 1H), 6.72 (d, J=4.3 Hz, 1H), 4.44 (s, 1H), 4.26 (t, J=7.0 Hz, 2H), 2.44 (s, 3H), 1.90 (t, J=7.0 Hz, 2H), 1.19 (s, 6H).

### Example 51: rac-(6-Chloro-imidazo[1,5-a]pyridin-5-yl)-[5-chloro-1-(4-methoxy-phenyl)-1H-[1,2,3]triazol-4-yl]-methanol

### Step 1: preparation of (5-amino-1-(4-methoxyphenyl)-1H-1,2,3-triazol-4-yl)methanol

To a cooled (−70° C.) solution of ethyl 5-amino-1-(4-methoxyphenyl)-1H-1,2,3-triazole-4-carboxylate (262 mg, 1.00 mmol, 1 eq) in THF (4.5 mL) is added diisobutylaluminum hydride solution (1.0 M in toluene, 5 mL, 5.00 mmol, 5 eq). The resulting orange suspension is stirred at −70° C. for 2 h to afford completion. The reaction is quenched with sat. sodium potassium tartrate solution and extracted with EtOAc. The combined org. layers are dried (MgSO₄), filtered and concentrated under reduced pressure to give the title compound as a beige solid (149 mg, 68%). LC-MS (acidic): t_{R}=0.49 min, [M+H]⁺=221.17.

### Step 2: Preparation of (5-chloro-1-(4-methoxyphenyl)-1H-1,2,3-triazol-4-yl)methanol

To a mixture of (5-amino-1-(4-methoxyphenyl)-1H-1,2,3-triazol-4-yl)methanol (179 mg, 0.813 mmol, 1 eq), copper(I) chloride (249 mg, 2.44 mmol, 3 eq) and copper(II) chloride anhydrous (328 mg, 2.44 mmol, 3 eq) in MeCN (2.4 mL) is added isopentyl nitrite (0.603 mL, 4.31 mmol, 5.3 eq) at 0° C. The resulting solution is stirred at RT for 48 h. The reaction is quenched with water and extracted with EtOAc. The combined org. layers are dried (MgSO₄), filtered and concentrated under reduced pressure to give the title product. LC-MS (acidic): t_{R}=0.67 min, [M+H]⁺=240.29.

### Step 3: Preparation of 5-chloro-1-(4-methoxyphenyl)-1H-1,2,3-triazole-4-carbaldehyde

Prepared following the procedure described for Example 39, step 3, using (5-chloro-1-(4-methoxyphenyl)-1H-1,2,3-triazol-4-yl)methanol. LC-MS (acidic): t_{R}=0.82 min, [M+H]⁺=238.25.

### Step 4: Preparation of rac-(5-chloro-1-(4-methoxyphenyl)-1H-1,2,3-triazol-4-yl)(6-chloro-3-(ethylthio)imidazo[1,5-a]pyridin-5-yl)methanol

Prepared following the procedure described for Reference example 2 using 6-chloro-3-(ethylthio)imidazo[1,5-a]pyridine and 5-chloro-1-(4-methoxyphenyl)-1H-1,2,3-triazole-4-carbaldehyde. LC-MS (acidic): t_{R}=0.85 min, [M+H]⁺=449.94.

### Step 5: Preparation of rac-(6-chloro-imidazo[1,5-a]pyridin-5-yl)-[5-chloro-1-(4-methoxy-phenyl)-1H-[1,2,3]triazol-4-yl]-methanol

Prepared following the procedure described for Reference example 2 using rac-(5-chloro-1-(4-methoxyphenyl)-1H-1,2,3-triazol-4-yl)(6-chloro-3-(ethylthio)imidazo[1,5-a]pyridin-5-yl)methanol. Purification by preparative HPLC (basic conditions) gives rac-(6-chloro-imidazo[1,5-a]pyridin-5-yl)-[5-chloro-1-(4-methoxy-phenyl)-1H-[1,2,3]triazol-4-yl]-methanol. LC-MS (QC): t_{R}=0.782 min, [M+H]⁺=390.2.

### Example 52: (R)-(6-Cyclopropyl-imidazo[1,5-a]pyrazin-5-yl)-{1-[2,5-difluoro-4-(3-fluoro-oxetan-3-ylmethoxy)-phenyl]-1H-[1,2,3]triazol-4-yl}-methanol

### Step 1: Preparation of 1-azido-2,5-difluoro-4-(methoxymethoxy)benzene

Prepared according to the procedure described for Example 7, step 1 using 2,5-difluoro-4-(methoxymethoxy)phenylboronic acid.

### Step 2 Preparation of (R)-(6-cyclopropylimidazo[1,5-a]pyrazin-5-yl)(1-(2,5-difluoro-4-(methoxymethoxy)phenyl)-1H-1,2,3-triazol-4-yl)methanol

Prepared following the procedure described for Example 7 using Intermediate Ba, (S)-1-(6-cyclopropylimidazo[1,5-a]pyrazin-5-yl)prop-2-yn-1-ol and 1-azido-2,5-difluoro-4-(methoxymethoxy)benzene. Purification by prepHPLC (basic conditions) to give (R)-(6-cyclopropylimidazo[1,5-a]pyrazin-5-yl)(1-(2,5-difluoro-4-(methoxymethoxy)phenyl)-1H-1,2,3-triazol-4-yl)methanol. LC-MS (acidic): t_{R}=0.73; [M+H]⁺=429.20.

### Step 3: preparation of (R)-4-(44(6-cyclopropylimidazo[1,5-a]pyrazin-5-yl)(hydroxy)methyl)-1H-1,2,3-triazol-1-yl)-2,5-difluorophenol

To a solution of (R)-(6-cyclopropylimidazo[1,5-a]pyrazin-5-yl)(1-(2,5-difluoro-4-(methoxymethoxy)phenyl)-1H-1,2,3-triazol-4-yl)methanol in EtOAc is added HCl in dioxane (4M, 3 eq) and the white suspension is stirred at RT until completion of the reaction. The suspension is filtered and concentrated under reduced pressure. Purification by prepHPLC (acidic conditions) to give (R)-4-(44(6-cyclopropylimidazo[1,5-a]pyrazin-5-yl)(hydroxy)methyl)-1H-1,2,3-triazol-1-yl)-2,5-difluorophenol. LC-MS (acidic): t_{R}=0.62 min, [M+H]⁺=385.19.

### Step 4: Preparation of (R)-(6-cyclopropyl-imidazo[1,5-a]pyrazin-5-yl)-{1-[2,5-difluoro-4-(3-fluoro-oxetan-3-ylmethoxy)-phenyl]-1H-[1,2,3]triazol-4-yl}-methanol

Prepared following the procedure described for Example 16 using (R)-4-(44(6-cyclopropylimidazo[1,5-a]pyrazin-5-yl)(hydroxy)methyl)-1H-1,2,3-triazol-1-yl)-2,5-difluorophenol and 3-(bromomethyl)-3-fluorooxetane. Purification by preparative HPLC (basic conditions) gives (R)-(6-cyclopropyl-imidazo[1,5-a]pyrazin-5-yl)-{1-[2,5-difluoro-4-(3-fluoro-oxetan-3-ylmethoxy)-phenyl]-1H-[1,2,3]triazol-4-yl}-methanol. LC-MS (QC): t_{R}=0.805 min, [M+H]⁺=473.3.

### Example 53: (R)-(6-Cyclopropyl-imidazo[1,5-a]pyrazin-5-yl)-{1-[2,5-difluoro-4-(oxetan-3-ylmethoxy)-phenyl]-1H-[1,2,3]triazol-4-yl}-methanol

Prepared following the procedure described for Example 52 using (R)-4-(4-((6-cyclopropylimidazo[1,5-a]pyrazin-5-yl)(hydroxy)methyl)-1H-1,2,3-triazol-1-yl)-2,5-difluorophenol and 3-(bromomethyl)-oxetane. Purification by preparative HPLC (basic conditions) gives (R)-(6-cyclopropyl-imidazo[1,5-a]pyrazin-5-yl)-{1-[2,5-difluoro-4-(oxetan-3-ylmethoxy)-phenyl]-1H-[1,2,3]triazol-4-yl}-methanol. LC-MS (QC): t_{R}=0.756 min, [M+H]⁺=455.3. ¹H NMR (500 MHz, DMSO) δ: 8.96 (s, 1H), 8.78 (d, J=1.0 Hz, 1H), 8.62 (s, 1H), 7.88 (dd, J₁=11.1 Hz, J₂=7.1 Hz, 1H), 7.78 (d, 1H), 7.60 (dd, J₁=12.0 Hz, J₂=7.5 Hz, 1H), 6.89 (s, 1H), 6.76-6.87 (m, 1H), 4.64-4.80 (m, 7H), 2.47-2.49 (m, 1H), 1.05-1.08 (m, 1H), 0.92-1.01 (m, 3H).

### Example 54: rac-(6-Cyclopropyl-imidazo[1,5-a]pyrazin-5-yl)-[1-(2,4-difluoro-phenyl)-5-methyl-1H-[1,2,3]triazol-4-yl]-methanol

To a solution of 1-azido-2,4-difluorobenzene (32.7 mg, 0.2 mmol, 1 eq) in tert-butyl methyl ether anhydrous (0.128 mL, 1.07 mmol, 5.373 eq) is added at RT, 1-propynylmagnesium bromide solution 0.5 M in THF (0.42 mL, 0.21 mmol, 1.05 eq). The mixture is stirred at RT for 3 h. A solution of 6-cyclopropylimidazo[1,5-a]pyrazine-5-carbaldehyde (37.4 mg, 0.2 mmol, 1 eq) in THF (0.3 mL) is then added and the mixture stirred at RT for 1 h. The mixture is diluted with water and AcOEt. The layers are separated and the aqueous phase is further extracted with EtOAc 2×. The combined organic layers are dried (MgSO₄), filtered and concentrated under reduced pressure. Purification by preparative HPLC (basic conditions) gives rac-(6-cyclopropyl-imidazo[1,5-a]pyrazin-5-yl)-[1-(2,4-difluoro-phenyl)-5-methyl-1H-[1,2,3]triazol-4-yl]-methanol. LC-MS (QC): tᵣ=0.757 min, [M+H]⁺=383.3. ¹H NMR (500 MHz, DMSO) δ: 8.96 (s, 1H), 8.66 (s, 1H), 7.77-7.81 (m, 2H), 7.70 (m, 1H), 7.35-7.39 (m, 1H), 6.86 (d, J=4.0 Hz, 1H), 6.64 (d, J=4.0 Hz, 1H), 2.38-2.42 (m, 1H), 2.35 (s, 3H), 1.01-1.05 (m, 1H), 0.86-0.98 (m, 3H).

### Example 55: rac-(6-Cyclopropyl-imidazo[1,5-a]pyrazin-5-yl)-[1-(2,5-difluoro-4-methoxy-phenyl)-5-methyl-1H-[1,2,3]triazol-4-yl]-methanol

Prepared following the procedure described for Example 54 using 1-azido-2,5-difluoro-4-methoxybenzene and 6-cyclopropylimidazo[1,5-a]pyrazine-5-carbaldehyde. Purification by prepHPLC (basic conditions) to give rac-(6-cyclopropyl-imidazo[1,5-a]pyrazin-5-yl)-[1-(2,5-difluoro-4-methoxy-phenyl)-5-methyl-1H-[1,2,3]triazol-4-yl]-methanol. LC-MS (QC): t_{R}=0.783 min; [M+H]⁺=413.3. ¹H NMR (500 MHz, DMSO) δ: 8.96 (s, 1H), 8.66 (s, 1H), 7.74-7.78 (m, 2H), 7.52 (dd, J₁=11.7 Hz, J₂=7.6 Hz, 1H), 6.84 (d, J=4.0 Hz, 1H), 6.63 (d, J=4.0 Hz, 1H), 3.96 (s, 3H), 2.36-2.43 (m, 1H), 2.34 (s, 3H), 1.00-1.07 (m, 1H), 0.86-0.99 (m, 3H).

### Example 56: 1-(4-{4-[(R)-(6-Cyclopropyl-imidazo[1,5-a]pyrazin-5-yl)-hydroxy-methyl]-[1,2,3]triazol-1-yl}-2,5-difluoro-phenoxy)-2-methyl-propan-2-ol

Prepared following the procedure described for Example 52 using (R)-4-(44(6-cyclopropylimidazo[1,5-a]pyrazin-5-yl)(hydroxy)methyl)-1H-1,2,3-triazol-1-yl)-2,5-difluorophenol and 1-bromo-2-methylpropan-2-ol. Purification by preparative HPLC (basic conditions) gives 1-(4-{4-[(R)-(6-cyclopropyl-imidazo[1,5-a]pyrazin-5-yl)-hydroxy-methyl]-[1,2,3]triazol-1-yl}-2,5-difluoro-phenoxy)-2-methyl-propan-2-ol. LC-MS (QC): t_{R}=0.772 min, [M+H]⁺=457.4. ¹H NMR (500 MHz, DMSO) δ: 8.96 (s, 1H), 8.75 (d, J=1.0 Hz, 1H), 8.62 (s, 1H), 7.78 (d, 2H), 7.53 (dd, J₁=12.2 Hz, J₂=7.5 Hz, 1H), 6.88 (s, 1H), 6.80 (s, 1H), 4.75 (s, 1H), 3.92 (s, 2H), 2.47 (m, 1H), 1.22 (s, 6H), 1.05-1.07 (m, 1H), 0.93-1.00 (m, 3H).

### Example 57: rac-(6-Cyclopropyl-imidazo[1,5-a]pyrazin-5-yl)-(1-(2-fluoro-phenyl)-5-methyl-1H-[1,2,3]triazol-4-yl-methanol

Prepared following the procedure described for Example 54 using 1-azido-2-fluorobenzene and 6-cyclopropylimidazo[1,5-a]pyrazine-5-carbaldehyde. Purification by prepHPLC (basic conditions) to rac-(6-Cyclopropyl-imidazo[1,5-a]pyrazin-5-yl)-[1-(2-fluoro-phenyl)-5-methyl-1H-[1,2,3]triazol-4-yl]-methanol. LC-MS (QC): t_{R}=0.722 min; [M+H]⁺=365.3. ¹H NMR (500 MHz, DMSO) δ: 8.97 (s, 1H), 8.67 (s, 1H), 7.78 (d, 1H), 7.66-7.72 (m, 2H), 7.57-7.61 (m, 1H), 7.46 (td, J₁=7.6 Hz, J₂=1.1 Hz, 1H), 6.86 (d, J=4.1 Hz, 1H), 6.64 (d, J=4.1 Hz, 1H), 2.38-2.43 (m, 1H), 2.35 (d, 3H), 1.01-1.05 (m, 1H), 0.88-1.01 (m, 3H).

### Reference Example 1 (Ref1): rac-(6-Cyclopropyl-imidazo[1,5-a]pyrazin-5-yl)-(5-phenyl-thiophen-3-yl)-methanol

To a solution of isopropylmagnesium chloride lithium chloride (1.3 M in THF, 0.14 mL, 0.18 mmol) is added a solution of 2-bromo-5-phenyl-thiophene (45 mg, 0.18 mmol) in THF (0.2 mL) at 0° C. The reaction mixture is stirred at 0° C. for 1 h. The mixture is then cooled to −20° C. and 6-cyclopropylimidazo[1,5-a]pyrazine-5-carbaldehyde (30 mg, 0.16 mmol) is added. The reaction mixture is allowed to warm up to RT and stirred at this temperature for 1 h. Sat. aq. NH₄Cl and EtOAc are added, the layers separated and the aq. layer extracted with EtOAc (2×). The combined org. extracts are dried (MgSO₄), filtered and concentrated under reduced pressure. The crude product is purified by preparative HPLC (basic conditions) to give rac-(6-cyclopropyl-imidazo[1,5-a]pyrazin-5-yl)-(5-phenyl-thiophen-3-yl)-methanol as a white solid. LC-MS (QC): t_{R}=1.065 min, [M+H]⁺=348.10. ¹H NMR (400 MHz, DMSO) δ: 8.98 (s, 1H), 8.52 (s, 1H), 7.78 (s, 1H), 7.60 (d, J=7.4 Hz, 2H), 7.29-7.41 (m, 4H), 7.05 (s, 1H), 6.88-6.90 (m, 2H), 2.47 (d, J=4.7 Hz, 1H), 0.94-1.07 (m, 4H).

### Reference Example 2 (Ref2): rac-(6-Chloro-imidazo[1,5-a]pyridin-5-yl)-(1-phenyl-1H-pyrazol-3-yl)-methanol

### Step 1: Preparation of rac-(6-chloro-3-(ethylthio)imidazo[1,5-a]pyridin-5-yl)(1-phenyl-1H-pyrazol-3-yl)methanol

To a solution of lithium diisopropylamide solution (1.0 M in THF/hexanes, 0.80 mL, 0.80 mmol) in THF (1.6 mL) is added a solution of 6-chloro-3-(ethylthio)imidazo[1,5-a]pyridine (85 mg, 0.40 mmol) in THF (1.6 mL) in a dropwise manner at −40° C. The reaction mixture is stirred at −40° C. for 25 min. The solution is then cooled down to −78° C. and 1-phenyl-1H-pyrazole-3-carbaldehyde (145 mg, 0.80 mmol) is added solid in one portion. The mixture is stirred at −78° C. for 5 min, at −40° C. for 30 min and is then allowed to reach RT overnight. Sat. aq. NH₄Cl and EtOAc are added, the layers separated and the aq. layer extracted with EtOAc (2×). The combined org. extracts are dried (MgSO₄), filtered and concentrated under reduced pressure to give rac-(6-chloro-3-(ethylthio)imidazo[1,5-a]pyridin-5-yl)(1-phenyl-1H-pyrazol-3-yl)methanol. LC-MS (acidic): t_{R}=0.84 min, [M+H]⁺=385.2.

### Step 2: Preparation of rac-(6-chloro-imidazo[1,5-a]pyridin-5-yl)-(1-phenyl-1H-pyrazol-3-yl)-methanol

To a solution of rac-(6-chloro-3-(ethylthio)imidazo[1,5-a]pyridin-5-yl)(1-phenyl-1H-pyrazol-3-yl)methanol (154 mg, 0.40 mmol) in EtOH (4 mL) is added Raney nickel. The resulting black suspension is stirred at 45° C. until completion of the reaction. The mixture is filtered and washed with DCM and EtOH and concentrated under reduced pressure. The crude product is purified by preparative HPLC (basic conditions) to give rac-(6-chloro-imidazo[1,5-a]pyridin-5-yl)-(1-phenyl-1H-pyrazol-3-yl)-methanol as an off-white solid. LC-MS (QC): t_{R}=0.760 min, [M+H]⁺=325.10. ¹H NMR (500 MHz, DMSO) δ: 8.57 (m, 1H), 8.49 (d, J=2.5 Hz, 1H), 7.66-7.68 (m, 2H), 7.58-7.63 (m, 1H), 7.42-7.46 (m, 3H), 7.27 (m, 1H), 6.86-6.90 (m, 1H), 6.80-6.82 (m, 1H), 6.72 (d, J=2.5 Hz, 1H), 6.69 (d, J=4.4 Hz, 1H).

### Reference Example 3 (Ref3): rac-(6-Chloro-imidazo[1,5-a]pyridin-5-yl)-(2-phenyl-2H-[1,2,3]triazol-4-yl)-methanol

### Step 1: Preparation of rac-(6-chloro-3-(ethylthio)imidazo[1,5-a]pyridin-5-yl)(2-phenyl-2H-1,2,3-triazol-4-yl)methanol

Prepared following the procedure described for Reference example 2 using 6-chloro-3-(ethylthio)imidazo[1,5-a]pyridine and 2-phenyl-2H-1,2,3-triazole-4-carbaldehyde. LC-MS (acidic): t_{R}=0.88 min, [M+H]⁺=385.85.

### Step 2: Preparation of rac-(6-chloro-imidazo[1,5-a]pyridin-5-yl)-(2-phenyl-2H-[1,2,3]triazol-4-yl)-methanol

Prepared following the procedure described for Reference example 2 using rac-(6-chloro-3-(ethylthio)imidazo[1,5-a]pyridin-5-yl)(2-phenyl-2H-1,2,3-triazol-4-yl)methanol. Purification by preparative HPLC (basic conditions) gives rac-(6-chloro-imidazo[1,5-a]pyridin-5-yl)-(2-phenyl-2H-[1,2,3]triazol-4-yl)-methanol. LC-MS (QC): t_{R}=0.833 min, [M+H]⁺=326.10. ¹H NMR (500 MHz, DMSO) δ: 8.57 (s, 1H), 8.34 (s, 1H), 7.84-7.89 (m, 2H), 7.64-7.69 (m, 1H), 7.48-7.55 (m, 3H), 7.35-7.43 (m, 1H), 7.11 (d, J=4.4 Hz, 1H), 6.92 (m, 1H), 6.80-6.87 (m, 1H).

### Reference Example 4 (Ref4): rac-(6-Chloro-imidazo[1,5-a]pyridin-5-yl)-(5-phenyl-thiophen-2-yl)-methanol

### Step 1: Preparation of rac-(6-chloro-3-(ethylthio)imidazo[1,5-a]pyridin-5-yl)(5-phenylthiophen-2-yl)methanol

Prepared following the procedure described for Reference example 2 using 6-chloro-3-(ethylthio)imidazo[1,5-a]pyridine and 5-phenylthiophene-2-carbaldehyde. LC-MS (acidic): t_{R}=0.99 min, [M+H]⁺=401.09.

### Step 2: Preparation of rac-(6-chloro-imidazo[1,5-a]pyridin-5-yl)-(5-phenyl-thiophen-2-yl)-methanol

Prepared following the procedure described for Reference example 2 using rac-(6-chloro-3-(ethylthio)imidazo[1,5-a]pyridin-5-yl)(5-phenylthiophen-2-yl)methanol. Purification by preparative HPLC (basic conditions) gives rac-(6-chloro-imidazo[1,5-a]pyridin-5-yl)-(5-phenyl-thiophen-2-yl)-methanol. LC-MS (QC): t_{R}=1.030 min, [M+H]⁺=341.00. ¹H NMR (500 MHz, DMSO) δ: 8.50-8.53 (m, 1H), 7.68 (dd, J=9.6 Hz, 1H), 7.58-7.63 (m, 2H), 7.50 (s, 1H), 7.37-7.43 (m, 2H), 7.35 (m, 1H), 7.28-7.32 (m, 1H), 7.25 (d, J=4.6 Hz, 1H), 6.91-6.95 (m, 2H), 6.79-6.81 (m, 1H).

The absolute chirality and the binding mode of the compound of Example 1a was determined by an X-ray diffraction analysis of the corresponding compound-enzyme co-crystals using the following experimental procedure:

1. Protein Purification and Co-Crystallization:

IDO1 protein was expressed and purified following a procedure described in the literature (Biochem et Biophysica Acta 1814 (2011) 1947-1954). IDO1 protein was concentrated to 29 mg/ml in a buffer containing 10 mM MES (2-(N-morpholino)ethanesulfonic acid) pH 6.50, 100 mM NaCl and 2 mM TCEP (Tris(2-carboxyethyl)phosphine hydrochloride). The protein solution was incubated with the compound of Example 1 at a final concentration of 2 mM for 3 hours at 277 K. The solution was then centrifuged for 5 minutes at 15,000 rpm at 277 K using an Eppendorf 5424R benchtop centrifuge. The centrifuged solution was mixed with a reservoir solution containing 30 mM lithium sulfate, 30 mM sodium sulfate, 30 mM potassium sulfate, 100 mM 3-morpholino-2-hydroxypropanesulfonic acid/bis(2-hydroxyethyl)amino-tris(hydroxymethyl)methane pH 6.5, 10% (w/v) PEG 8000 and 20% (w/v) 1,5-pentanediol. Co-crystals of IDO1 and the compound of Example 1a were finally obtained by vapour diffusion from sitting drops at 293 K.

2. X-Ray Data Collection and Structure Determination:

The above-mentioned co-crystals were harvested using nylon loops and placed directly in liquid nitrogen. Synchrotron data were collected at beamline X06DA of the Swiss Light Source at the Paul Scherrer Institute, Villigen, Switzerland using a Pilatus 2M-F detector. Diffraction images were processed using the program XDS (Acta Cryst. (2010) D66, 125-132). The preliminary structure was solved using the program Phaser (J. Appl. Cryst. (2007) 40, 658-674). Refinement and rebuilding of the structure were carried out using the programs Refmac5 (Acta Cryst. (2004) D60, 2284-2295) and Coot (Acta Cryst. (2010) D66, 486-501), respectively. R-free was calculated using a randomly selected 5% of total data from the observed reflections. Based on the measured electron density, it was unambiguously established that the compound of Example 1a is the (R)-enantiomer.

Data Collection and Refinement Statistics

| | |
|---|---|
| Final resolution (Å) | 2.25 |
| Space group | P2₁2₁2₁ |
| Unit cell dimensions (Å) | a = 84.3, b = 92.1, c = 132.3 |
| Wavelength (Å) | 1.0000 |
| observed/unique reflections | 331660/94277 |
| Resolution range (Å)^{a} | 46.07-2.25 (2.39-2.25) |
| Completeness (%) | 99.6 (99.0) |
| Rmerge (%)^{b} | 11.4 (134.1) |
| I/σ (l) | 9.32 (0.84) |
| Refinement | |
| Rwork (%) | 22.9 |
| Rfree (%) | 25.7 |
| RMSD | |
| bond length (Å) | 0.008 |
| bond angle (°) | 1.3 |
| Ramachandran outliers | 0 |

^{a}values shown in parentheses correspond to the highest resolution shell
${}^{b} R = \frac{{\sum }_{hkl} {\sum }_{j} \vert {I}_{hkl , j} - 〈 {I}_{hkl} 〉 \vert}{{\sum }_{hkl} {\sum }_{j} {I}_{hkl , j}}$

(R)- or (S)-configuration of the compounds according to the present invention is assigned to the compounds of Examples 2a, 3a, 7a, 8a, 10a, 11a, 20a, 21-31, 33, 34a, 35a, 36, 40-42, 44-47, 52, 53 and 56 based on the assumption that the binding mode of the more active enantiomer is the same as the one for the compound of Example 1.

### Biological Tests

1) Testing Compounds for IDO Inhibitory Activity in an IDO1 Enzymatic Assay:

Recombinant full-length human IDO1 with a N-terminal hexahistidine tag expressed in E. coli and purified to homogeneity is incubated at a final concentration of 2 nM in assay buffer consisting of 37.5 mM phosphate buffer at pH6.5 supplemented with 10 mM ascorbic acid, 0.45 μM methylene blue, 50 U/ml catalase, 0.01% BSA, and 0.01% Tween 20 (protocol modified from Seegers et al, JBS 2014). Example compounds are serially diluted in DMSO, further diluted in phosphate buffer, and added to the enzyme at final concentrations ranging from 10 μM to 0.5 nM. The final DMSO concentration is 0.6%. Following a pre-incubation of 30 minutes at RT, the reaction is started by the addition of L-tryptophan at a final concentration of 5 μM in assay buffer. After 30 minutes of incubation at RT, 3 μL of the 20 μl reaction mixture are transferred to a 384 deep well plate containing 25 μL of deionized water. 100 μl of 200 nM L-Tryptophan-(indole-d5) in cold 100% methanol are added followed by a 10 minutes centrifugation at 3220×g at 4° C. An additional 75 μL of deionized water are then added followed by a 10 minutes centrifugation at 3220×g at 4° C. The product of the reaction N′-Formylkynurenine (NFK) is quantified by LCMS and normalized to the L-Tryptophan-(indole-d5) signal. Samples with 0.6% DMSO (0% effect) and a TDO/IDO inhibitor (100% effect) are used as control samples to set the parameters for the non-linear regression necessary for the determination of the half-maximal inhibitory concentration (IC50) for each compound. For each compound concentration the percentage of activity compared to 0% and 100% effect is calculated as average±STDEV (each concentration measured in duplicate). IC50 values and curves are generated with XLfit software (IDBS) using Dose-Response One Site model 203 (four parameter logistic curve model). The calculated IC50 values may fluctuate depending on the daily assay performance. Fluctuations of this kind are known to those skilled in the art. When compounds are measured multiple times, mean values are given.

2) Testing Compounds for TDO Inhibitory Activity in a TDO2 Enzymatic Assay:

Recombinant human TDO comprising amino acids 19-407 with a N-terminal hexahistidine tag expressed in E. coli and purified to homogeneity is incubated at a final concentration of 15 nM in assay buffer consisting of 75 mM phosphate buffer at pH7 supplemented with 100 μM ascorbic acid, 50 U/ml Catalase, 0.01% BSA, and 0.01% Tween 20 (protocol modified from Seegers et al, JBS 2014). Example compounds are serially diluted in DMSO, further diluted in phosphate buffer, and added to the reaction mixture at final concentrations ranging from 10 μM to 0.5 nM. The final DMSO concentration is 0.6%. Following a pre-incubation of 30 minutes at RT, the reaction is started by the addition of L-tryptophan at a final concentration of 200 μM in assay buffer. After 30 minutes of incubation at RT, 3 μL of the reaction mixture are transferred to a 384 deep well plate containing 25 μL of deionized water. 100 μl of 200 nM L-Tryptophan-(indole-d5) in cold 100% methanol are added followed by a 10 minutes centrifugation at 3220×g at 4° C. An additional 75 μL of deionized water are then added followed by a 10 minutes centrifugation at 3220×g at 4° C. The product of the reaction N′-Formylkynurenine (NFK) is quantified by LCMS and normalized to the L-Tryptophan-(indole-d5) signal. Samples with 0.6% DMSO (0% effect) and a TDO/IDO inhibitor (100% effect) are used as control samples to set the parameters for the non-linear regression necessary for the determination of the half-maximal inhibitory concentration (IC50) for each compound. For each compound concentration the percentage of activity compared to 0% and 100% effect is calculated as average±STDEV (each concentration measured in duplicate). IC50 values and curves are generated with XLfit software (IDBS) using Dose-Response One Site model 203 (four parameter logistic curve model). The calculated IC50 values may fluctuate depending on the daily assay performance. Fluctuations of this kind are known to those skilled in the art. When compounds are measured multiple times, mean values are given.

3) Testing Compounds for IDO/TDO Inhibitory Activity and Toxicity in Cell-Based Assays

SW48 cells (ATCC, CCL-231) are used to measure compounds for TDO inhibitory activity and are routinely maintained in DMEM high glucose/GlutaMAX™/pyruvate 90% (v/v), FCS 10% (v/v), Penicilin/streptomycin 1% (v/v). SKOV3 cells (NCI, No. 0503405) which upregulate IDO1 after stimulation with IFNγ are used to measure compounds for IDO inhibitory activity. SKOV3 cells are routinely maintained in RPMI 90% (v/v), FCS 10% (v/v), Penicilin/streptomycin 1% (v/v). SW48 or SKOV3 cells are seeded in 384 well plates at a density of 8000 cells in 45 ul per well or 4000 cells in 45 ul per well, respectively. Plates are incubated at 37° C./5% CO₂ for 24 hours. On the next day, 10 ul compound in serial dilutions (tested concentration range 10 uM-40 nM) and 200 uM L-tryptophan are added (SKV03 receive in addition IFNγ at a final concentration of 50 ng/ml). After 24 hours of incubation at 37° C./5% CO₂, 3 ul of the supernatant per well is transferred to 25 ul H₂O per well in a 384 deep well plate and 25 ul of the supernatant per well is transferred to waste. The SKOV3 and SW48 cell plates with 25 ul supernatant per well remaining are used to measure viability (see below). The 384 deep well plate containing 3 ul supernatant and 25 ul H₂O per well are further processed for LCMS: After the addition of 100 ul L-tryptophan-(indole-d5) (Sigma 615862) at 200 nM in methanol, the 384 deep well plates are centrifuged for 10 minutes at 3220×g at 4° C., 75 ul H₂O is added per well and plates centrifuged again for 10 minutes at 3220×g at 4° C. N-formylkynurenine and kynurenine are quantified by LCMS, normalized to the internal standard L-tryptophan-(indole-d5) and the sum is calculated. Samples with 0.2% DMSO (0% effect) and a TDO/IDO inhibitor (100% effect) are used as control samples to set the parameters for the non-linear regression necessary for the determination of the IC50 for each compound. For each compound concentration the percentage of activity compared to 0% and 100% effect is calculated as average±STDEV (each concentration measured in duplicate). IC50 values and curves are generated with XLfit software (IDBS) using Dose-Response One Site model 203. The calculated IC50 values may fluctuate depending on the daily cellular assay performance. Fluctuations of this kind are known to those skilled in the art. When compounds are measured multiple times, mean values are given.

As inhibition of NFK and KYN production can simply be an effect of cytotoxicity, a viability assay (CellTiter-Glo 2.0Luminescent Cell Viability Assay, Promega Catalog #G9243) is performed in parallel. CellTiter-Glo reagent is added (25 ul/well) to cell plates, incubated for 15 minutes at room temperature in the dark and luminescence is measured with the EnVision Multilabel Reader from Perkin Elmer according to manufacturer's instructions.

The luminescent signal is proportional to the amount of ATP present. The amount of ATP is directly proportional to the number of viable cells present. Samples with 0.2% DMSO (0% effect) and a toxic compound (100% effect) are used as control samples to set the parameters for the non-linear regression. For each compound concentration the percentage of activity compared to 0% and 100% effect is calculated as average±STDEV (each concentration measured in duplicate). Tox IC50 values and curves are generated with XLfit software (IDBS) using Dose-Response One Site model 203. The calculated IC50 values may fluctuate depending on the daily cellular assay performance. Fluctuations of this kind are known to those skilled in the art. When compounds are measured multiple times, mean values are given.

The results of biological tests 1, 2 and 3 obtained for the compounds of Examples 1 to 57 and Reference examples 1 to 4 are summarized in Table 1 below.

| | hIDO | SKOV3 | hTDO |
|---|---|---|---|
| | activity | activity | activity |
| Example | (IC₅₀ in | (IC₅₀ in | (IC₅₀ in |
| Number | nM) | nM) | nM) |
| 1 | 9.60 | 246 | >10200 |
| 1a | 5.07 | 193 | 9110 |
| 2 | 6.17 | 225 | >10200 |
| 2a | 4.29 | 84.9 | >10200 |
| 3 | 41.0 | 470 | >10200 |
| 3a | 16.7 | 282 | >10200 |
| 4 | 3.70 | 42.2 | >10200 |
| 5 | 4.83 | 53.7 | >10200 |
| 6 | 9.14 | 11.8 | >10200 |
| 7 | 3.77 | 6.12 | >10200 |
| 7a | 2.12 | 4.84 | >10200 |
| 7b | 361 | 703 | >10200 |
| 8 | 7.41 | 8.80 | >10200 |
| 8a | 9.85 | 18.9 | >10200 |
| 9 | 5.43 | 31.2 | >10200 |
| 10 | 9.60 | 25.4 | >10200 |
| 10a | 2.93 | 12.5 | >10200 |
| 11 | 12.5 | 37.1 | >10200 |
| 11a | 8.17 | 23.7 | >10200 |
| 12 | 21.5 | 73.9 | >10200 |
| 13 | 27.2 | 326 | >10200 |
| 14 | 5.05 | 34.6 | >10200 |
| 15 | 13.1 | 208 | 9610 |
| 16 | 33.2 | 94.6 | >10200 |
| 17 | 20.4 | 172 | >10200 |
| 18 | 33.2 | 140 | >10200 |
| 19 | 13.7 | 11.6 | >10200 |
| 20 | 2.78 | 14.5 | 5260 |
| 20a | 1.84 | 10.7 | 2190 |
| 21 | 8.24 | 33.2 | >10200 |
| 22 | 6.36 | 20.2 | >10200 |
| 23 | 9.61 | 51.9 | |
| 24 | 8.36 | 399 | >10200 |
| 25 | 5.86 | 33.0 | >10200 |
| 26 | 4.50 | 74.3 | >10200 |
| 27 | 5.60 | 47.4 | >10200 |
| 28 | 6.28 | 19.5 | >10200 |
| 29 | 7.74 | 29.9 | >10200 |
| 30 | 3.68 | 52.4 | 8600 |
| 31 | 1.13 | 9.20 | >10200 |
| 32 | 14.3 | 70.2 | >10200 |
| 33 | 5.33 | 127 | >10200 |
| 34 | 2.97 | 14.5 | >10200 |
| 34a | 1.82 | 7.92 | >10200 |
| 35 | 3.39 | 3.05 | >10200 |
| 35a | 2.39 | 0.95 | >10200 |
| 36 | 8.33 | 16.8 | >10200 |
| 37 | 5.28 | 39.0 | 6640 |
| 38 | 5.43 | 86.0 | 4710 |
| 39 | 5.26 | 6.94 | 7940 |
| 40 | 16.5 | 154 | >10200 |
| 41 | 17.5 | 212 | >10200 |
| 42 | 13.5 | 199 | >10200 |
| 43 | 3.88 | 64.2 | 832 |
| 44 | 14.5 | 377 | >10200 |
| 45 | 15.0 | 56.8 | >10200 |
| 46 | 15.1 | 356 | >10200 |
| 47 | 7.76 | 145 | >10200 |
| 48 | 2.29 | 29.0 | 1460 |
| 49 | | 10.2 | 8360 |
| 50 | | 5.41 | 8200 |
| 51 | | 145 | 7040 |
| 52 | | 18.1 | >10200 |
| 53 | | 27.4 | >10200 |
| 54 | | 364 | >10200 |
| 55 | | 35.0 | >10200 |
| 56 | | 29.3 | >10200 |
| 57 | | 333 | >10200 |
| Ref1 | 189 | 3830 | >10200 |
| Ref2 | 433 | 2940 | >10200 |
| Ref3 | 161 | >10000 | 8920 |
| Ref4 | 275 | 8830 | 8650 |

1. A compound according to Formula (I) 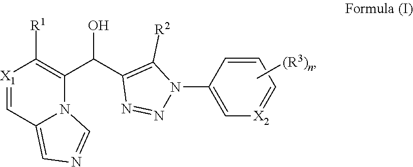
wherein
X₁ represents nitrogen or carbon;
X₂ represents nitrogen or carbon;
R¹ represents
C₁₋₄-alkyl;
C₃₋₅-cycloalkyl; or
halogen;
R² represents
hydrogen;
C₁₋₃-alkyl; or
halogen;
each R³ independently represents
C₁₋₄-alkyl;
C₁₋₃-alkoxy-C₁₋₄-alkyl;
halogen;
—OR⁴, wherein R⁴ represents hydrogen, C₁₋₄-alkyl, hydroxy-C₂₋₅-alkyl, (oxetan-3-yl)-C₁₋₃-alkyl, or (3-fluoro-oxetan-3-yl)-C₁₋₃-alkyl;
—NR^{N1}R^{N2}, wherein
R^{N1} represents hydrogen and R^{N2} represents —(C═O)—R^{CO}, wherein R^{CO} represents C₁₋₃-alkoxy;
R^{N1} and R^{N2} independently represent hydrogen or C₁₋₃-alkyl;
R^{N1} and R^{N2}, together with the nitrogen atom to which they are attached, form a 4- to 6-membered saturated heterocyclic ring comprising one nitrogen ring atom; or
R^{N1} represents C₁₋₃-alkyl and R^{N2} represents 1,2-ethanediyl such that the fragment 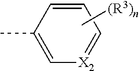
of Formula (I) represents 1-(C₁₋₃-alkyl)-2,3-dihydro-indol-5-yl;
2-oxa-6-aza-spiro[3.3]hept-6-yl or 6-oxa-1-aza-spiro[3.3]hept-1-yl; and
n represents 0, 1, 2, 3, 4 or 5;
or a pharmaceutically acceptable salt thereof.

2. A compound according to claim 1, wherein
X₁ represents nitrogen or carbon;
X₂ represents nitrogen or carbon;
R¹ represents
C₁₋₄-alkyl;
C₃₋₅-cycloalkyl; or
halogen;
R² represents
hydrogen; or
C₁₋₃-alkyl;
each R³ independently represents
C₁₋₄-alkyl;
C₁₋₃-alkoxy-C₁₋₄-alkyl;
halogen;
—OR⁴, wherein R⁴ represents hydrogen, C₁₋₄-alkyl, hydroxy-C₂₋₅-alkyl, (oxetan-3-yl)-C₁₋₃-alkyl, or (3-fluoro-oxetan-3-yl)-C₁₋₃-alkyl; or
—NR^{N1}R^{N2}, wherein R^{N1} represents hydrogen and R^{N2} represents —(C═O)—R^{CO}, wherein R^{CO} represents C₁₋₃-alkoxy; and
n represents 0, 1, 2, 3, 4 or 5;
or a pharmaceutically acceptable salt thereof.

3. A compound according to claim 1, wherein X₁ represents carbon; or a pharmaceutically acceptable salt thereof.

4. A compound according to claim 1, wherein X₂ represents carbon; or a pharmaceutically acceptable salt thereof.

5. A compound according to claim 1, wherein R¹ represents C₃₋₅-cycloalkyl or halogen; or a pharmaceutically acceptable salt thereof.

6. A compound according to claim 1, wherein R² represents hydrogen or C₁₋₃-alkyl; or a pharmaceutically acceptable salt thereof.

7. A compound according to claim 1, wherein
n represents 1, 2 or 3;
one substituent R³ represents
—OR⁴, wherein R⁴ represents hydrogen, C₁₋₄-alkyl, hydroxy-C₂₋₅-alkyl, (oxetan-3-yl)-C₁₋₃-alkyl or (3-fluoro-oxetan-3-yl)-C₁₋₃-alkyl; or
—NR^{N1}R^{N2}, wherein
R^{N1} represents hydrogen and R^{N2} represents —(C═O)—R^{CO}, wherein R^{CO} represents C₁₋₃-alkoxy;
R^{N1} and R^{N2} independently represent hydrogen or C₁₋₃-alkyl;
R^{N1} and R^{N2}, together with the nitrogen atom to which they are attached, form a 4- to 6-membered saturated heterocyclic ring comprising one nitrogen ring atom; or
2-oxa-6-aza-spiro[3.3]hept-6-yl or 6-oxa-1-aza-spiro[3.3]hept-1-yl;
wherein said one substituent is attached in para-position with regard to the point of attachment to the rest of the molecule and the remaining R³, if present, is/are selected from halogen;
or a pharmaceutically acceptable salt thereof.

8. A compound according to claim 1, wherein
n represents 1, 2 or 3;
one substituent R³ represents
—OR⁴, wherein R⁴ represents hydrogen, C₁₋₄-alkyl, hydroxy-C₂₋₅-alkyl, (oxetan-3-yl)-C₁₋₃-alkyl or (3-fluoro-oxetan-3-yl)-C₁₋₃-alkyl; or
—NR^{N1}R^{N2}, wherein R^{N1} represents hydrogen and R^{N2} represents —(C═O)—R^{CO}, wherein R^{CO} represents C₁₋₃-alkoxy;
wherein said one substituent is attached in para-position with regard to the point of attachment to the rest of the molecule and the remaining R³, if present, is/are selected from halogen; or a pharmaceutically acceptable salt thereof.

9. A compound according to claim 1, wherein the fragment 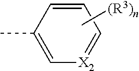
of Formula (I) represents
phenyl, 4-hydroxyphenyl, 4-methoxyphenyl, 3-bromo-4-methoxyphenyl, 4-methylphenyl, 3-chloro-4-hydroxyphenyl, 3-chloro-4-methoxyphenyl, 3-fluoro-4-hydroxyphenyl, 3-fluoro-4-methoxyphenyl, 2-fluoro-3-chloro-4-methoxyphenyl, 3-chloro-4-methoxy-5-fluorophenyl, 2-fluoro-4-methoxy-5-chlorophenyl, 2,5-difluoro-4-methoxyphenyl, 4-((oxetan-3-yl)methoxy)-phenyl, 3-fluoro-4-((oxetan-3-yl)methoxy)-phenyl, 4-((3-fluoro-oxetan-3-yl)methoxy)-phenyl, 3-fluoro-4-((3-fluoro-oxetan-3-yl)methoxy)-phenyl, 4-(methoxy-carboxamido)-phenyl, 4-(2-hydroxy-2-methylpropoxy)-phenyl, 4-(methoxymethyl)-phenyl; or 4-ethoxypyridin-3-yl; or, in addition to the above-listed, 3-fluoro-4-(2-hydroxy-2-methylpropoxy)-phenyl, or 6-ethoxypyridin-3-yl; or
3-fluoro-4-(3-hydroxy-3-methylbutoxy)-phenyl, 3-chloro-4-(3-hydroxy-3-methylbutoxy)-phenyl, 2,5-difluoro-4-((3-fluoro-oxetan-3-yl)methoxy)-phenyl, 2,5-difluoro-4-((oxetan-3-yl)methoxy)-phenyl, 2,5-difluoro-4-(2-hydroxy-2-methylpropoxy)-phenyl, 4-(2-oxa-6-aza-spiro[3.3]hept-6-yl)-phenyl, 4-(6-oxa-1-aza-spiro[3.3]hept-1-yl)-phenyl, 1-methyl-2,3-dihydro-1H-indol-5-yl, 4-amino-phenyl, 4-(methylamino)-phenyl, 4-(pyrrolidin-1-yl)-phenyl, 4-dimethylamino-phenyl, 2-fluoro-phenyl, or 2,4-difluoro-phenyl;
or a pharmaceutically acceptable salt thereof.

10. A compound according to claim 1, which are also compounds of Formula (II) 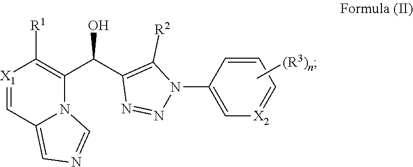
or a pharmaceutically acceptable salt thereof.

11. A compound according to claim 1 selected from a group consisting of
(6-Chloro-imidazo[1,5-a]pyridin-5-yl)-(1-phenyl-1H-[1,2,3]triazol-4-yl)-methanol;
(R)-(6-Chloro-imidazo[1,5-a]pyridin-5-yl)-(1-phenyl-[1,2,3]triazol-4-yl)-methanol;
(6-Methyl-imidazo[1,5-a]pyridin-5-yl)-(1-phenyl-1H-[1,2,3]triazol-4-yl)-methanol;
(R)-(6-Methyl-imidazo[1,5-a]pyridin-5-yl)-(1-phenyl-[1,2,3]triazol-4-yl)-methanol;
(6-Cyclopropyl-imidazo[1,5-a]pyrazin-5-yl)-(1-phenyl-[1,2,3]triazol-4-yl)-methanol;
(R)-(6-Cyclopropyl-imidazo[1,5-a]pyrazin-5-yl)-(1-phenyl-1H-[1,2,3]triazol-4-yl)-methanol;
(6-Cyclopropyl-imidazo[1,5-a]pyridin-5-yl)-[1-(4-methoxy-phenyl)-[1,2,3]triazol-4-yl]-methanol;
(6-Cyclopropyl-imidazo[1,5-a]pyridin-5-yl)-(1-p-tolyl-1H-[1,2,3]triazol-4-yl)-methanol;
(4-{4-[(6-Cyclopropyl-imidazo[1,5-a]pyridin-5-yl)-hydroxy-methyl]-[1,2,3]triazol-1-yl}-phenyl)-carbamic acid methyl ester;
2-Chloro-4-{4-[(6-cyclopropyl-imidazo[1,5-a]pyridin-5-yl)-hydroxy-methyl]-[1,2,3]triazol-1-yl}-phenol;
2-Chloro-4-{4-[(R)-(6-cyclopropyl-imidazo[1,5-a]pyridin-5-yl)-hydroxy-methyl]-[1,2,3]triazol-1-yl}-phenol;
2-Chloro-4-{4-[(S)-(6-cyclopropyl-imidazo[1,5-a]pyridin-5-yl)-hydroxy-methyl]-[1,2,3]triazol-1-yl}-phenol;
[1-(3-Chloro-4-methoxy-phenyl)-1H-[1,2,3]triazol-4-yl]-(6-cyclopropyl-imidazo[1,5-a]pyridin-5-yl)-methanol;
(R)-[1-(3-Chloro-4-methoxy-phenyl)-1H-[1,2,3]triazol-4-yl]-(6-cyclopropyl-imidazo[1,5-a]pyridin-5-yl)-methanol;
(6-Cyclopropyl-imidazo[1,5-a]pyridin-5-yl)-[1-(6-ethoxy-pyridin-3-yl)-1H-[1,2,3]triazol-4-yl]-methanol;
2-Chloro-4-{4-[(6-cyclopropyl-imidazo[1,5-a]pyrazin-5-yl)-hydroxy-methyl]-[1,2,3]triazol-1-yl}-phenol;
2-Chloro-4-{4-[(R)-(6-cyclopropyl-imidazo[1,5-a]pyrazin-5-yl)-hydroxy-methyl]-[1,2,3]triazol-1-yl}-phenol;
[1-(3-Chloro-4-methoxy-phenyl)-1H-[1,2,3]triazol-4-yl]-(6-cyclopropyl-imidazo[1,5-a]pyrazin-5-yl)-methanol;
(R)-[1-(3-Chloro-4-methoxy-phenyl)-1H-[1,2,3]triazol-4-yl]-(6-cyclopropyl-imidazo[1,5-a]pyrazin-5-yl)-methanol;
(4-{4-[(6-Cyclopropyl-imidazo[1,5-a]pyrazin-5-yl)-hydroxy-methyl]-[1,2,3]triazol-1-yl}-phenyl)-carbamic acid methyl ester;
(6-Cyclopropyl-imidazo[1,5-a]pyrazin-5-yl)-[1-(6-ethoxy-pyridin-3-yl)-1H-[1,2,3]triazol-4-yl]-methanol;
4-{4-[(6-Cyclopropyl-imidazo[1,5-a]pyridin-5-yl)-hydroxy-methyl]-[1,2,3]triazol-1-yl}-phenol;
4-{4-[(6-Cyclopropyl-imidazo[1,5-a]pyrazin-5-yl)-hydroxy-methyl]-[1,2,3]triazol-1-yl}-phenol;
(6-Cyclopropyl-imidazo[1,5-a]pyrazin-5-yl)-{1-[4-(3-fluoro-oxetan-3-ylmethoxy)-phenyl]-1H-[1,2,3]triazol-4-yl}-methanol;
(6-Cyclopropyl-imidazo[1,5-a]pyrazin-5-yl)-[1-(4-methoxy-phenyl)-1H-[1,2,3]triazol-4-yl]-methanol;
1-(4-{4-[(6-Cyclopropyl-imidazo[1,5-a]pyrazin-5-yl)-hydroxy-methyl]-[1,2,3]triazol-1-yl}-phenoxy)-2-methyl-propan-2-ol;
(6-Cyclopropyl-imidazo[1,5-a]pyridin-5-yl)-{1-[4-(3-fluoro-oxetan-3-ylmethoxy)-phenyl]-1H-[1,2,3]triazol-4-yl}-methanol;
(6-Chloro-imidazo[1,5-a]pyridin-5-yl)-[1-(4-methoxy-phenyl)-5-methyl-1H-[1,2,3]triazol-4-yl]-methanol;
(R)-(6-Cyclopropyl-imidazo[1,5-a]pyrazin-5-yl)-[1-(3-fluoro-4-methoxy-phenyl)-1H-[1,2,3]triazol-4-yl]-methanol;
(R)-(6-Cyclopropyl-imidazo[1,5-a]pyrazin-5-yl)-[1-(2,5-difluoro-4-methoxy-phenyl)-1H-[1,2,3]triazol-4-yl]-methanol;
(R)-[1-(3-Bromo-4-methoxy-phenyl)-1H-[1,2,3]triazol-4-yl]-(6-cyclopropyl-imidazo[1,5-a]pyrazin-5-yl)-methanol;
(R)-(6-Cyclopropyl-imidazo[1,5-a]pyrazin-5-yl)-[1-(4-methoxymethyl-phenyl)-1H-[1,2,3]triazol-4-yl]-methanol;
(R)-[1-(5-Chloro-2-fluoro-4-methoxy-phenyl)-1H-[1,2,3]triazol-4-yl]-(6-cyclopropyl-imidazo[1,5-a]pyrazin-5-yl)-methanol;
(R)-[1-(3-Chloro-5-fluoro-4-methoxy-phenyl)-1H-[1,2,3]triazol-4-yl]-(6-cyclopropyl-imidazo[1,5-a]pyrazin-5-yl)-methanol;
4-{4-[(R)-(6-Cyclopropyl-imidazo[1,5-a]pyrazin-5-yl)-hydroxy-methyl]-[1,2,3]triazol-1-yl}-2-fluoro-phenol;
(R)-(6-Cyclopropyl-imidazo[1,5-a]pyrazin-5-yl)-{1-[3-fluoro-4-(3-fluoro-oxetan-3-ylmethoxy)-phenyl]-1H-[1,2,3]triazol-4-yl}-methanol;
1-(4-{4-[(R)-6-Cyclopropyl-imidazo[1,5-a]pyrazin-5-yl)-hydroxy-methyl]-[1,2,3]triazol-1-yl}-2-fluoro-phenoxy)-2-methyl-propan-2-ol; phenoxy)-2-methyl-propan-2-ol;
(R)-(6-Chloro-imidazo[1,5-a]pyridin-5-yl)-[1-(4-methoxy-phenyl)-1H-[1,2,3]triazol-4-yl]-methanol;
(R)-(6-Chloro-imidazo[1,5-a]pyridin-5-yl)-[1-(2,5-difluoro-4-methoxy-phenyl)-1H-[1,2,3]triazol-4-yl]-methanol;
[1-(2,5-Difluoro-4-methoxy-phenyl)-1H-[1,2,3]triazol-4-yl]-(6-ethyl-imidazo[1,5-a]pyrazin-5-yl)-methanol;
(R)-[1-(3-Chloro-2-fluoro-4-methoxy-phenyl)-1H-[1,2,3]triazol-4-yl]-(6-cyclopropyl-imidazo[1,5-a]pyrazin-5-yl)-methanol;
(6-Ethyl-imidazo[1,5-a]pyridin-5-yl)-[1-(4-methoxy-phenyl)-1H-[1,2,3]triazol-4-yl]-methanol;
(R)-(6-Ethyl-imidazo[1,5-a]pyridin-5-yl)-[1-(4-methoxy-phenyl)-1H-[1,2,3]triazol-4-yl]-methanol;
[1-(2,5-Difluoro-4-methoxy-phenyl)-1H-[1,2,3]triazol-4-yl]-(6-ethyl-imidazo[1,5-a]pyridin-5-yl)-methanol;
(R)-[1-(2,5-Difluoro-4-methoxy-phenyl)-1H-[1,2,3]triazol-4-yl]-(6-ethyl-imidazo[1,5-a]pyridin-5-yl)-methanol; and
(R)-(6-Cyclopropyl-imidazo[1,5-a]pyrazin-5-yl)-{1-[3-fluoro-4-(oxetan-3-ylmethoxy)-phenyl]-1H-[1,2,3]triazol-4-yl}-methanol;
or a pharmaceutically acceptable salt thereof.

12. A compound according to claim 1 selected from a group consisting of
(6-Chloro-imidazo[1,5-a]pyridin-5-yl)-[5-ethyl-1-(4-methoxy-phenyl)-1H-[1,2,3]triazol-4-yl]-methanol;
(6-Chloro-imidazo[1,5-a]pyridin-5-yl)-(5-methyl-1-phenyl-1H-[1,2,3]triazol-4-yl)methanol;
[1-(5-Chloro-2-fluoro-4-methoxy-phenyl)-5-methyl-1H-[1,2,3]triazol-4-yl]-(6-chloro-imidazo[1,5-a]pyridin-5-yl)-methanol;
(R)-(6-Cyclopropyl-imidazo[1,5-a]pyrazin-5-yl)-[1-(4-pyrrolidin-1-yl-phenyl)-1H-[1,2,3]triazol-4-yl]-methanol;
(R)-[1-(4-Amino-phenyl)-1H-[1,2,3]triazol-4-yl]-(6-cyclopropyl-imidazo[1,5-a]pyrazin-5-yl)-methanol;
(R)-(6-Cyclopropyl-imidazo[1,5-a]pyrazin-5-yl)-[1-(4-methylamino-phenyl)-1H-[1,2,3]triazol-4-yl]-methanol;
(R)-(6-Chloro-imidazo[1,5-a]pyridin-5-yl)-[1-(4-methoxy-phenyl)-5-methyl-1H-[1,2,3]triazol-4-yl]-methanol;
2-Chloro-4-{4-[(6-chloro-imidazo[1,5-a]pyridin-5-yl)-hydroxy-methyl]-5-methyl-[1,2,3]triazol-1-yl}-phenol;
(R)-(6-Cyclopropyl-imidazo[1,5-a]pyrazin-5-yl)-[1-(4-dimethylamino-phenyl)-1H-[1,2,3]triazol-4-yl]-methanol;
(R)-(6-Cyclopropyl-imidazo[1,5-a]pyrazin-5-yl)-{1-[4-(2-oxa-6-aza-spiro[3.3]hept-6-yl)-phenyl]-1H-[1,2,3]triazol-4-yl}-methanol;
(R)-(6-Cyclopropyl-imidazo[1,5-a]pyrazin-5-yl)-{1-[4-(6-oxa-1-aza-spiro[3.3]hept-1-yl)-phenyl]-1H-[1,2,3]triazol-4-yl}-methanol;
(R)-(6-Cyclopropyl-imidazo[1,5-a]pyrazin-5-yl)-[1-(1-methyl-2,3-dihydro-1H-indol-5-yl)-1H-[1,2,3]triazol-4-yl]-methanol;
4-{4-[(6-Chloro-imidazo[1,5-a]pyridin-5-yl)-hydroxy-methyl]-5-methyl-[1,2,3]triazol-1-yl}-2-fluoro-phenol;
4-(2-Chloro-4-{4-[(6-chloro-imidazo[1,5-a]pyridin-5-yl)-hydroxy-methyl]-5-methyl-[1,2,3]triazol-1-yl}-phenoxy)-2-methyl-butan-2-ol;
4-(4-{4-[(6-Chloro-imidazo[1,5-a]pyridin-5-yl)-hydroxy-methyl]-5-methyl-[1,2,3]triazol-1-yl}-2-fluoro-phenoxy)-2-methyl-butan-2-ol;
(6-Chloro-imidazo[1,5-a]pyridin-5-yl)-[5-chloro-1-(4-methoxy-phenyl)-1H-[1,2,3]triazol-4-yl]-methanol;
(R)-(6-Cyclopropyl-imidazo[1,5-a]pyrazin-5-yl)-{1-[2,5-difluoro-4-(3-fluoro-oxetan-3-ylmethoxy)-phenyl]-1H-[1,2,3]triazol-4-yl}-methanol;
(R)-(6-Cyclopropyl-imidazo[1,5-a]pyrazin-5-yl)-{1-[2,5-difluoro-4-(oxetan-3-ylmethoxy)-phenyl]-1H-[1,2,3]triazol-4-yl}-methanol;
(6-Cyclopropyl-imidazo[1,5-a]pyrazin-5-yl)-[1-(2,4-difluoro-phenyl)-5-methyl-1H-[1,2,3]triazol-4-yl]-methanol;
(6-Cyclopropyl-imidazo[1,5-a]pyrazin-5-yl)-[1-(2,5-difluoro-4-methoxy-phenyl)-5-methyl-1H-[1,2,3]triazol-4-yl]-methanol;
1-(4-{4-[(R)-(6-Cyclopropyl-imidazo[1,5-a]pyrazin-5-yl)-hydroxy-methyl]-[1,2,3]triazol-1-yl}-2,5-difluoro-phenoxy)-2-methyl-propan-2-ol; and
(6-Cyclopropyl-imidazo[1,5-a]pyrazin-5-yl)-[1-(2-fluoro-phenyl)-5-methyl-1H-[1,2,3]triazol-4-yl]-methanol;
or a pharmaceutically acceptable salt thereof.

13. A pharmaceutical composition comprising a compound according to claim 1, or a pharmaceutically acceptable salt thereof, and further comprising at least one pharmaceutically acceptable carrier.

14. (canceled)

15. A method for prevention and/or treatment of cancer, wherein the method comprises administering a compound according to claim 1 or a pharmaceutically acceptable salt thereof.

16. A pharmaceutical composition comprising a compound according to claim 11, or a pharmaceutically acceptable salt thereof, and further comprising at least one pharmaceutically acceptable carrier.

17. A pharmaceutical composition comprising a compound according to claim 12, or a pharmaceutically acceptable salt thereof, and further comprising at least one pharmaceutically acceptable carrier.

18. A method for prevention and/or treatment of cancer, wherein the method comprises administering a compound according to claim 11 or a pharmaceutically acceptable salt thereof.

19. A method for prevention and/or treatment of cancer, wherein the method comprises administering a compound according to claim 12 or a pharmaceutically acceptable salt thereof.
